(19) United States
(12) Patent Application Publication
(10) Pub. No.: US 2012/0076808 A1
(43) Pub. Date: Mar. 29, 2012

(54) COMBINED ANTIGEN AND DNA VACCINE FOR PREVENTING AND TREATING AUTOIMMUNE DISEASES

(75) Inventors: Bin WANG, Beijing (CN); Shuang GENG, Shanghai (CN); Jin JIN, Beijing (CN)

(73) Assignee: CHINA AGRICULTURAL UNIVERSITY, Beijing (CN)

(21) Appl. No.: 13/245,930

(22) Filed: Sep. 27, 2011

Related U.S. Application Data
(60) Provisional application No. 61/386,839, filed on Sep. 27, 2010.
(60) Provisional application No. 61/466,741, filed on Mar. 23, 2011.

(51) Int. Cl. A61K 39/00; A61P 37/00; A61P 37/08; A61P 11/06; A61P 21/04; A61P 25/00; A61P 9/10; A61P 29/00; A61P 5/14; A61K 39/35; A61P 3/10

(52) U.S. Cl. 424/185.1

(57) ABSTRACT

The present invention relates to treating and preventing symptoms of an allergy, asthma, an autoimmune disease, and transplant rejection using a combination vaccine containing an antigen and a DNA encoding the antigen.

### CROSS REFERENCE TO RELATED APPLICATIONS

This application claims the benefit of U.S. Provisional Application No. 61/386,839, filed on Sep. 27, 2010, and U.S. Provisional Application No. 61/466,741 filed on Mar. 23, 2011, the contents of each of which are incorporated herein by reference.

### SEQUENCE LISTING

The instant application contains a Sequence Listing which has been submitted in ASCII format and is hereby incorporated by reference in its entirety. Said ASCII copy, created on Sep. 23, 2011, is named VGX0119W.txt.

### FIELD OF THE INVENTION

The present invention relates to treating and preventing symptoms of an allergy, asthma, an autoimmune disease, and transplant rejection using a vaccine containing an antigen and a DNA encoding the antigen.

### BACKGROUND OF THE INVENTION

Regulatory T (Treg) cells are important regulators of tolerance, which plays an important role in autoimmune disease treatments. Specifically, inducing antigen-specific Treg (iTreg) cells targeted to allergy, asthma, and autoimmune disease antigens offers a promising immunomodulatory treatment strategy for the associated conditions. A known approach for providing Treg cells is adoptive transfer of naturally occurring thymus-derived CD4⁺CD25⁺ Treg (nTreg) cells. This approach, however, yields low levels of islet-specific Treg cells among the nTreg cells, and consequently inefficient suppression.

Inducible regulatory T (iTreg) cells are generated from conventional CD4⁺ T cells through tolerogenic antigen presentation in the periphery. In contrast naturally regulatory T (nTreg) cells, tolerogenic antigen presentation can be induced by co-immunization using a protein antigen and a DNA vaccine encoding the same antigen. Simultaneous exposure to the combination of protein- and DNA-based antigens generates CD40^{low} IL-10^{high} dendritic cells, which mediate induction of CD4⁺CD25⁻Foxp3⁺ iTerg cells in an antigen specific manner. These iTregs would be useful for suppressing Th₁- and Th₂-induced immune pathways such as allergies, autoimmune diseases, asthma, and transplant rejection.

It is not known, however, how to design antigenic epitopes or vaccines for antigen presentation so as to maximize the induction of iTreg. Accordingly, there is a need in the art for better methods of antigen selection and design for antigen-based vaccines against autoimmune diseases, and for effective vaccines against such diseases, including effective routes of administration of same.

### SUMMARY OF THE INVENTION

Provided herein is a vaccine comprising an antigenic peptide and a DNA encoding the peptide. The antigenic peptide and DNA stimulate iTreg cells. The antigen may be associated with a condition, such as an allergy, asthma or an autoimmune disease. The antigen may be a dermatophagoides pteronyssinus 1 peptide, a fragment thereof, or a variant thereof and may be associated with an allergy or asthma. The antigen may be an insulin peptide, myelin oligodendrocyte glycoprotein, myelin basic protein, and oligodendrocyte-specific protein, zonapellucida protein peptide, dermatophagoides pteronyssinus 1 peptide, α-myosin peptide, coxsackievirus B4 structural protein peptide, group A streptococcal M5 protein peptide, (Q/R)(K/R) RAA, type II collagen peptide, thyroid peroxidase, thyroglobulin, pendrin peptide, acetylcholine receptor peptide, human S-antigen, a fragment thereof, or a variant thereof, and may be associated with an autoimmune disease. A vector may comprise the DNA encoding the peptide. The vector may be a pVAX, pcDNA3.0, or a provax vector. The vector and antigenic peptide may be at a mass ratio of 5:1 and 1:5; or 1:1 and 2:1.

Also provided herein is a vaccination kit. The vaccination kit may contain a vaccine administration device and the herein described vaccine. The vaccination device may be a vaccine gun, a needle, or an electroporation device.

Also provided herein is a method of treating an autoimmune disease. The method may comprise administering the herein described vaccine to a patient in need thereof. The autoimmune disease may be type I diabetes mellitus, multiple sclerosis, autoimmune ovarian disease, dust mite allergy, myocarditis rheumatoid arthritis, thyroiditis, myasthenia gravis, autoimmune uveitis, or asthma. The antigen of the vaccine may be an insulin peptide, a fragment thereof, or a variant thereof if the vaccine is to be used in treating type I diabetes mellitus. The antigen of the vaccine may be a myelin oligodendrocyte glycoprotein, myelin basic protein, an oligodendrocyte-specific protein, a fragment thereof, or a variant thereof if the vaccine is to be used in treating multiple sclerosis. The antigen of the vaccine may be a zonapellucida protein peptide, a fragment thereof, or a variant thereof if the vaccine is to be used in treating an autoimmune ovarian disease. The antigen of the vaccine may be a dermatophagoides pteronyssinus 1 peptide, a fragment thereof, or a variant thereof if the vaccine is to be used in treating myocarditis. The antigen of the vaccine may be an α-myosin peptide, coxsackievirus B4 structural protein peptide, group A streptococcal M5 protein peptide, a fragment thereof, or a variant thereof if the vaccine is to be used in myocarditis. The antigen of the vaccine may be a peptide (Q/R)(K/R) RAA, type II collagen peptide, a fragment thereof, or a variant thereof if the vaccine is to be used in treating rheumatoid arthritis. The antigen of the vaccine may be a thyroid peroxidase, thyroglobulin, pendrin peptide, a fragment thereof, or a variant thereof if the vaccine is to be used in treating thyroiditis. The antigen of the vaccine may be an acetylcholine receptor peptide, a fragment thereof, or a variant thereof if the vaccine is to be used in treating myasthenia gravis. The antigen of the vaccine may be a human S-antigen, a fragment thereof, or a variant thereof if the vaccine is to be used in treating autoimmune uveitis.

### BRIEF DESCRIPTION OF THE DRAWINGS

FIG. 1 shows that MHC-II blocking reduces CD25⁻ iTreg induction. Purified CD4⁺ T cells from Balb/c DO11.10 mice or OVA (Ovalbumin)₃₂₃₋₃₃₉-sensitized Balb/c mice were cultured with purified tolerogenic dendritic cells (DCs) from co-immunized Balb/c mice, in the presence or absence of anti-MHC-II blocking mAb. CD25⁻ iTreg cells (CD4⁺CD25⁻Foxp3⁺) were counted on day 7 as percentage of CD4⁺CD25⁻ T cells*, p<0.05. Shown is one of three independent experiments with similar results. Each dot represents one mouse.

FIG. 2 shows that OVA₃₂₃₋₃₃₉ mutations reduce antigenicity for T cells. FIG. 2A. Summary of OVA₃₂₃₋₃₃₉ mutations, their predicted MHC-II binding affinities, and experimental result from tetramer competition assays. Percent of tetramer binding was calculated as: number of tetramer-positive T cells in the presence of a competing peptide epitope/number of tetramer-positive T cells in the absence of a competing peptide epitope×100%. FIG. 2B. Proliferation of CFSE-labeled DO11.10 CD4⁺ T cells co-cultured for 4 days with tolerogenic dendritic cells (“DCs”) presenting an indicated epitope. The line plots summarize the results from three independent experiments. **, p<0.01.

FIG. 3 shows induction of CD25⁻ iTreg cells by co-immunization depends on epitope affinity. Fig A. CD25⁻ iTreg (CD4⁺CD25⁻Foxp3⁺ (Foxhead Box P3)) and nTreg (CD4⁺CD25⁺Foxp3⁺), induced in Balb/c mice following co-immunization, were counted by flow cytometry and calculated as percentage of Foxp3⁺ cells in CD4⁺CD25⁻ and CD4⁺CD25⁺ T cells, respectively. Naïve, non-immunized mice. **, p<0.01. Each point represents one mouse. Shown is one of three independent experiments with similar results. FIG. 3B. Induction of highly suppressive CD25⁻ iTreg cells by co-immunization depends on epitope antigenecity. CFSE labeled DO11.10 CD4⁺ T cells were co-cultured with co-immunization-induced CD25⁻ iTreg, in the presence of OVA₃₂₃₋₃₃₉. Proliferation was determined by flow cytometry as divided KJ1-26⁺ cells versus total KJ1-26⁺ cells. **, p<0.01. Each point represents one mouse. Shown is one of three independent experiments with similar results.

FIG. 4 shows that adoptive transfer of CD25⁻ iTreg cells suppresses T cell response in recipient mice. CD4⁺CD25⁻ T cells from OVA₃₂₃₋₃₃₉, MT1, or MT2 co-immunized, or from naïve Balb/c, were adoptively transferred to naïve Balb/c. The activity of the donor CD25⁻ iTreg was assessed by sensitizing the recipients with OVA₃₂₃₋₃₃₉ in IFA. FIG. 4A. CD4⁺ T cells were isolated from the recipient after sensitization. The cells were labeled with carboxy-fluorescein succinimidylester (CFSE) and restimulated with OVA₃₂₃₋₃₃₉ in culture. Divided cells were identified by CFSE dilution and counted by flow cytometry. The result is expressed as a percent of total CFSE⁺ T cells. Shown is one of three independent experiments of similar results. FIG. 4B. CD4⁺ T cells were isolated from the recipients after sensitization and intracellularly immunostained for IFN-γ. IFN-γ′CD4⁺ T cells were counted by flow cytometry and calculated as a percent of total CD4⁺ T cells. Shown is one of three independent experiments of similar results. FIGS. 4C and D. IFN-γ and IL-10 secretion in the supernatant of restimulated T cells. Anti-CD3 mAb (KT3) or KT3+IL-2+IL-4 was used in positive controls for induction of indicated cytokines. Shown is one of three independent experiments of similar results. *, p<0.05, **, p<0.01.

FIG. 5 shows that P100 stimulates T cells more strongly than P66. Splenic CD4⁺ T cells from flea antigen immunized C57BL/6 mice were restimulated with P100 or P66 (5 ug/ml) in culture. T cell proliferation was determined by a 3-(4,5-Dimethylthiazol-2-yl)-2,5-diphenyltetrazolium bromide (MTT, a yellow tetrazole)-based assay. Concanavalin A (1 ug/ml) and BSA (1 ug/ml) were used as positive and negative controls, respectively. *, p<0.05. Shown is one of three independent experiments with similar results.

FIG. 6 shows that attenuation of skin reaction by co-immunization-induced CD25⁻ iTreg. FIG. 6A. Flea antigen stimulated T cell proliferation. FIG. 6B. In vivo T cell response induced by flea-specific i.d. test. FIG. 6C. H&E staining of skin section. The black arrows indicate infiltrating T cells. FIG. 6D. Mast cell number and degranulation (black arrow) by Toluidine Blue staining FIG. 6E. Seven days after co-immunization, CD25⁻ iTreg cells were counted as a percentage of CD4⁺CD25⁻ T cells. Shown is one of three independent experiments with similar results. *, p<0.05; **, p<0.01.

FIG. 7 shows adoptive transfer of CD25⁻ iTreg suppresses skin response in vivo. CD25⁻ iTreg from Co100 or Co66 immunized mice were adoptively transferred into FSA1-sensitized mice. The recipients were then challenged with flea antigens (skin test). Histamine and PBS were used as positive and negative controls for the skin test, respectively. *, p<0.05. Shown is one of three independent experiments with similar results.

FIG. 8. Co-immunization suppresses development of HDM-mediated asthma. (A) Histological examination of lung tissues by H&E staining 24 hrs after the last challenge with dust mite extracts. The arrows show different cell infiltrations (white arrow). (B) Levels of IgE specific to Der-p1 are tested by ELISA 24 hrs after the last challenging. (C) Different cytokine levels in the serum of mice 24 hrs after the last challenge are examined by Flex set. *, p<0.05 **, p<0.01 compared with the model group, n=6 mice per group.

FIG. 9. Co-immunization induces CD4⁺CD25⁻Foxp3⁺ iTregs. (A) Foxp3 expression in CD4⁺CD25⁻ T cells on days 7 after the second co-immunization is analyzed by a FACS. (B-C) Inhibition of iTregs (purified from Foxp3 mice pretreated with co-immunization) is examined by co-culturing with responder T cells (CD4⁺ T cells purified from WT mice pretreated with Der-p1 stimulation) at a 1:5 or 1:10 ratio in the present of APC and stimulator for 72 hrs. The proliferation level is analyzed by a MTT method. Results are representative of at least three independent experiments. *, p<0.05, **, p<0.01 mismatched control or naïve groups as indicated, n=6 mice per group.

FIG. 10. Suppressive capacity of iTregs is mediated by IL-10, but not cell-cell contact. (A) iTregs and nTregs are analyzed for the expression of suppressive receptors on days 7 after the second co-immunization by fluorescence activated cell sorting (FACS). (B) In the transwell plate, 2×10⁵ freshly isolated CD4+CD25⁻GFP⁺ (green fluorescent protein) T cells were stimulated to secrete cytokines by Derp1 antigen (10 μm/ml) in upper chambers. 1×10⁶ responder CD4+ T cells were stimulated by Derp1 antigen (10 μg/ml) to expand in lower chambers. 10 μm/mL of control IgG, anti-IL-10 or anti-TGF-β was added as indicated in lower chambers. The proliferation level is analyzed by MTT method. Results are representative of at least three independent experiments. *, p<0.05, **, p<0.01 mismatched control or naïve groups as indicated, n=6 mice per group.

FIG. 11. TGF-β1 is necessary for induction of Foxp3 expression in iTregs. (A) Cytokine production in CD11C⁺ dendritic cells from the spleen of mice on days 3 after the first co-immunization is examined by RT-PCR. The expression of glyceraldehyde 3-phosphate dehydrogenase (GAPDH) is served as an internal control of samples. (B) Forkhead Box P3 (Foxp3) expression in CD4⁺CD25⁻ T cells when blocking the TGF-β1 in vivo. Mice are injected intralesionally for three consecutive days with anti-TGF-β Ab alone (400 μg/injection) or isotype control antibody mouse immunoglobulin G1 (IgGl) alone 3 times after each co-immunization. GFP expression analyzed by FACS 7 days after the second co-immunization. Results are representative of at least three independent experiments. *, p<0.05, **, p<0.01 mismatched control or naïve groups as indicated, n=6 mice per group.

FIG. 12. IL-10 is important for suppressive capacity of iTregs. (A) CD4⁺CD25⁻ Foxp3⁺ iTregs could also be induced when blocking the IL-10 in vivo. Foxp3 expression in CD4⁺CD25⁻ T cells was analyzed by FACS. (B) The suppressive ability of iTregs induced under deficiency of IL-10 were demolished. iTregs isolated from mice pretreated with anti-IL-10 mAb were cocultured with responder T cells. The proliferation level is analyzed by MTT method. (C) The level of IL-10 secreted by iTreg after treated with anti-TGF-b or anti-IL-10 mAb were evaluated by FACS. Results are representative of at least three independent experiments. *, p<0.05, **, p<0.01 mismatched control or naïve groups as indicated, n=6 mice per group.

FIG. 13. TGF-β1 induces Foxp3 expression in CD4⁺CD25⁻ naïve T cells in vitro. (A) The model of TGF-β and IL-10 in Dcreg induces iTreg. (B) Naive T cells were cocultured with dendritic cells (DC) reg co-treated with DNA and Der-p1 protein for 7 days, the Foxp3-GFP was evaluated by FACS. (C) Naïve CD4⁺CD25⁻ T cells purified from Foxp3^{gfp} mice were stimulated with plate bound anti-CD3 and soluble anti-CD28 in the presence of different doses of TGF-β1 for 72 hrs and assessed for the expression of GFP by FACS. (D) CD4⁺CD25⁻ T cells were stimulated and cultured as (C) in the presence of TGF-β1 or IL-10 for 72 hrs and assessed for the expression of GFP by FACS. Results are representative of at least three independent experiments. *, p<0.05, **, p<0.01 mismatched control or naïve groups as indicated, n=6 mice per group.

FIG. 14. Autocrine IL-10 has an effect on iTreg suppressive capacity. (A) Naive T cells were cocultured with DC pre-treated with both of DNA and dermatophagoides P1 protein (Der-p1) Der-p1 protein or single antigen respectively for 7 days. The Foxp3-GFP was then evaluated by FACS. (B) iTreg isolated from medium as (A) and cocultured with effector T cells to evaluate its suppressive capacity. (C) IL-10R expression on CD11⁺ DC surface was detected on different days after pretreated with DNA and protein vaccine by FACS. (D) Naive T cells were cocultured with DC pre-treated with both of DNA, Der-p1 protein and IL-10R siRNA. The iTreg induction was evaluated by FACS. (E) The iTreg cells were isolated from medium as (D) and cocultured with effector T cells to evaluate its suppressive capacity. The proliferation level is analyzed by MTT method. Results are representative of at least three independent experiments. *, p<0.05, **, p<0.01 mismatched control or naïve groups as indicated, n=6 mice per group.

FIG. 15. The model of TGF-b and IL-10 function was studied and it related to induction of iTreg by co-immunization. FIG. 15A shows nuclear or cytoplasmic nuclear factor of activated T-cells 1 and 2 (NFAT1 and NFAT2) by Western blot in purified CD4⁺CD25⁻GFP⁺ iTregs and CD4+CD25+GFP+ nTregs. Histone or GAPDH was used as loading controls for nuclear or cytoplasmic protein, respectively. (B) The TGF-β and IL-10 have an effect on different stages of co-immunization.

FIG. 16. Analysis of expression of pVAX-Der-p1 in eukaryotic and prokaryotic expressing constructs. (A) RNA isolated from transfected baby hamster kidney cells (BHK21 cells) with pVAX-Der-p1 is analyzed by RT-PCR with Der-p1 specific primers. Lane 1, a DNA marker; Lane 2, RNA from the transfected BHK21 cells; Lanes 3, RNA from the transfected pVAX vector BHK21 cells; Lanes 4, RNA from the non-transfected BHK21 cells. A determination of expression of the Der-p1 protein in E. coli system was conducted via SDS PAGE (B) and Western blot (C). SDS PAGE results 1: Uninduced pET28a-Der-p1; 2: Induced pET28a-Der-p1 with 0.1 mM IPTG; 3: Induced pET28a-Der-p1 with 0.5 mM IPTG; 4: Induced pET28a-Der-p1 with 1.5 mM IPTG; 5: Protein molecular weight standards. Arrow points at target band. (C) Western blot results 1: Uninduced pET28a-Der-p1; 2: Induced pET28a-Der-p1.

FIG. 17. Analysis of different cells in BAL. 24 h after the last challenge, BAL is collected and the number of infiltrating cells (total) and eosinophils assessed by CELL-DYN. Results are representative of three experiments. *p<0.05 **, p<0.01 compared with the model group. (n=6 cats per group)

FIG. 18. Co-immunization up-regulates GFP expression in CD4⁺CD25⁻ T cells derived from Foxp3 mice. GFP expression in CD4+CD25− T cells on days 7 after the second co-immunization is analyzed by a FACS. Results are representative of at least three independent experiments.

FIG. 19. Level of TGF-(31 or IL-10 in mouse serum after treated with mAb. (A) TGF-β1 levels in the sera of mice on days 3 after the second co-immunization is examined by ELISA kit. (B) IL-10 levels in the sera of mice on days 3 after the second co-immunization is examined by Flex Set. Results are representative of at least three independent experiments. *, p<0.05 **, p<0.01 compared with the model group, n=6 mice per group.

FIG. 20. The TGF-β receptor inhibitor suppresses the Foxp3 induction. Naive T cells were cocultured with DC pre-treated with both of DNA, Der-p1 protein and TGF-(3 receptor. The iTreg induction were evaluated by FACS. Results are representative of at least three independent experiments.

FIG. 21. IL-10 has no effect on the stage of Treg induction by DCreg. iTreg were induced by DCreg with anti-IL-10, and then were isolated after 7 days. Suppressive function of these iTreg were evaluated by proliferation level of effector T cells. The proliferation level is analyzed by MTT method. Results are representative of at least three independent experiments.

FIG. 22. The effect of IL-10R siRNA on DC was studied. The level of IL-10R on DC surface were performed on day 2 after treated with IL-10R siRNA or control siRNA. Results are representative of at least three independent experiments.

FIG. 23. CD40low is a marker for co-immunization-induced DCregs. A) Mice were injected i.m. with indicated immunogens. Spleen DCs were examined next day by double-staining for CD11c and CD40-PE, followed by flow cytometry. CD11c+ cells were gated. Naïve mice were used as the negative control. Shown are independent experiments with similar results. B) Purified CD11c+DCs and JAWS II cells were fed indicated immunogens for 24 h and expression of CD40 was examined by flow cytometry. Untreated DCs or JAWS II cells were used as the negative control. C) JAWS II cells were fed pOVA323+OVA323 or pVAX+OVA323 for 24 h and then co-cultured for 5 d with CFSE-CD4+ T cells prepared from mice that had been sensitized for OVA. Expression of Foxp3 and IL-10 was analyzed by FACS. CD4+ cells were gated. Count of Foxp3+ or IL-10+ cells was calculated as percentages of the gated cells. Shown are independent experiments with similar results. D) JAWS II cells were fed fluorescently labeled immunogens as indicated for 24 h and then immunostained for CD40. The correlation between uptake of the immunogens and expression of CD40 was analyzed by confocal microscopy (top panel). Mean PE-fluorescence was analyzed using the Nikon EZ-C1 3.00 FreeViewer software (bottom panel). Cell number is 10/group.

FIG. 24. DCs co-take up DNA and protein immunogens via clathrin- and caveolae-mediated endocytosis. A) JAWS II cells were pre-treated with PBS, MDC (50 μM), or filipin (10 μg/ml) for 30 min at 37° C. and then fed Cy5-pOVA323+FITC-OVA323 or Cy5-pVAX+FITC-OVA323 for 24 h. The cells were stained with anti-CD40-PE and analyzed by flow cytometry. Shown is CD40 staining of Cy5/FITC double-positive cells (gated). B) A summary of the experimental results are shown.

FIG. 25. Co-immunization activates negative pathways mediated by Cav-1. Total protein or RNA was extracted from spleen DCs of naïve mice or mice immunized with indicated immunogens 2 days before the analysis. Western blot (A, C, and D) and RT-PCR analyses were performed for the indicated proteins and genes.

FIG. 26. Silencing Cav-1 and Tollip prevents the induction of DCregs. A) WT and Cav-1 and/or Tollip knockdown DCs were fed pOVA323+OVA323 or pVAX+OVA323 for 24 h and expression of CD40 and IL-10 was analyzed. WT DCs not fed any immunogens were used control (Non-treated). B) DCs fed pOVA323+OVA323 or pVAX+OVA323 for 24 h were co-cultured with CFSE-CD4+ T cells from mice sensitized for OVA. T cell proliferation and the number of Foxp3+ and IL-10+ T cells were determined.

FIG. 27. Cav-1- and/or Tollip-deficient DCs are not tolerogenic in vivo. Cav-1- and/or Tollip-deficient JAWS II cells were adoptively transferred into syngeneic mice (day 0). The mice were then immunized with OVA in IFA on days 0 and 7. On day 14, DTH response was tested. On day 15, T cell proliferation, expression of Foxp3 in T cells, and IL10 levels in supernatant were determined.

FIG. 28. Co-immunization-induced DCregs ameliorate inflammatory bronchitis. A) Experimental design: Balb/c mice were injected with 0.1 ml of 1 mg/ml OVA/alum complexes in PBS on days 0 and 7 by i.p. and subsequently challenged with 100 μg OVA intra-tracheally on days 14, 16 and 18 to establish the “model”. Control mice were received with PBS intra-tracheally on days 14, 16 and 18 and designated as the “shame” control. On day 21, 5×105 of CD11c+ cells from syngeneic donor mice were transferred into model mice once daily for 3 consecutive days by i.v. (n=3 per group). Prior to the transfer, donor CD11c+ cells purified from spleen of naïve mice were pre-treated with or without filipin and subsequently co-treated with pOVA+OVA or pVAX+OVA for 24 h. On day 14 after the final transfer, serum samples were taken to analyze the levels of IgE or cytokine productions. Sections of lung tissues were made to evaluate disease severity. B) the level of antigen specific IgE was analyzed by ELISA following adoptive transfer of indicated DCs. C) the production of IL-4 and IL-5 was examined by CBA before and the transfer of indicated DCs. D) Lung sections were examined by H&E staining and recorded under a light microscope at ×100 and ×200 magnification.

FIG. 29. Co-immunization-induced DCregs ameliorate autoimmune ovarian disease. A) Experimental design: C57BL/6 mice were injected with mZP3 protein emulsified in CFA at footpads to induce the AOD. After 14 d, 5×105 of JAWS II cells were transferred into these induced AOD mice once daily for 3 consecutive days by i.v. (n=6 per group). Prior to the transfer, the JAWS II cells were fed pcD-mZP3+mZP3 or pcD-OVA+mZP3 for 24 h, followed by Mitomycin C treatment (50 μg/ml) for 20 min at 37° C. On day 14 after the final transfer, serum was taken to analyze cytokine production and ovaries were fixed and sectioned for evaluation of disease severity. B) Production of IFN-γ, TNF-α and IL-5 was analyzed by CBA. Shown are experiments with similar results. *, C) Degree of disease was assessed by pathological analysis of tissue sections from each animal. Each dot in the plot represents one animal. D) On day 14 after the final transfer, splenocytes of each recipient group were triple-stained for CD4, Foxp3 and IL-10 and analyzed by flow cytometry. CD4+ cells were gated.

FIG. 30. Effect of amiloride on the expression of CD40 in JAWS II cells. JAWS II cells were pre-treated with amiloride (5 mM) for 10 min at 37° C. and then co-treated with Cy5-pOVA323+FITC-OVA323 or Cy5-pVAX+FITC-OVA323 for 24 h. The cells were stained with anti-CD40-PE and analyzed by flow cytometry.

FIG. 31. Regulation of Cav-1 and Tollip in JAWS II cells. JAWS II cells were fed the indicated immunogens for 24 h. Total protein or RNA was then extracted and analyzed by Western blot (A) or RT-PCR (B).

FIG. 32. Silence of Cav-1 and Tollip by RNAi. A) JAWS II cells were transfected with Cav-1 or Tollip specific siRNAs. At 24 h, the mRNA level of Cav-1 and Tollip was detected by real-time RT-PCR. B) WT and Cav-1 knockdown DCs were fed pOVA+OVA or pVAX+OVA for 24 h. Translocation of NF-κB was detected by Western blot.

FIG. 33. Histological examination of ovarian tissues on day 14 after the final adoptive DC transfer. Samples were viewed under a light microscope at ×40 and ×100 magnification. Solid arrows indicate ovarian follicles without inflammatory cell infiltrations; open arrows indicate ovarian follicles with inflammatory cell infiltrations.

FIG. 34 shows maps of plasmid expression vectors encoding influenza nucleoprotein (“NP”) and M2 antigens and the corresponding linear expression cassettes. The linear expression cassette perNP or perM2 contain CMV promoter, intron for splicing, full length gene of NP or M2 with stop codon and polyadenylation signal.

### DETAILED DESCRIPTION

The current invention relates to the discovery that iTreg cells are efficiently induced against specific antigens by administering a combination of the antigen and a DNA vaccine that encodes the antigen. This induction is far better than either the antigen or the DNA vaccine alone. The invention also relates to the discovery that the efficiency of iTreg cell induction can be enhanced further if the antigen has a high affinity for MHC Class II expressed on tolerogenic dendritic cells (DC). The provided vaccines contain a combination of a peptide antigen with high affinity for MHC Class II and a DNA expressing the same peptide, and these vaccines can induce an iTreg population capable of suppressing autoimmune diseases and allergies. The iTreg-inducing treatment is associated with far fewer side effects than other methods of treatment because the iTreg cells are antigen specific and therefore more effectively suppress antigen-specific T cell function.

Provided herein are vaccines comprising an antigenic peptide and a DNA encoding the peptide. The peptide may have an IC₅₀ of 100 nM, and can have an IC₅₀ of 50 nM or less for MHC Class II. The MHC class II can be expressed on a tolerogenic dendritic cell. The DNA can comprise an expression vector capable of expressing the peptide. The vector can be selected from among available vectors in the field, and may include pVAX, pcDNA3.0, or provax. The peptide may be an amino acid sequence contained in a protein selected from the group consisting of insulin, FSA1, Der-p1, myelin oligodendrocyte glycoprotein (MOG), myelin basic protein (MBP), proteolipid protein (PLP), myelin-associated oligodendrocyte basic protein (MOBP), oligodendrocyte-specific protein (OSP), glucose-6-phosphatase, zona pellucida 1, 2, or 3, human myosin, Coxsackievirus B4 structural protein VP1, VP2, VP3, or VP4, group A streptococcal M5 protein, type II collagen, thyroid peroxidase, thyroglobulin, Pendrin, acetylcholine receptor alpha subunit, human S-antigen, and human IRBP. The insulin peptide may comprise the amino acid sequence MRLLPLLALLA (SEQ ID NO:5) or SHLVEALYLVCGERG (SEQ ID NO:191). The MOG peptide may comprise an amino acid sequence selected from the group consisting of HPIRALVGDEVELP (SEQ ID NO:36), VGWYRPPFSRVVHLYRNGKD (SEQ ID NO:37), LKVEDPFYWVSPGVLVLLAVLPVLLL (SEQ ID NO:38), MOG1-22 (SEQ ID NO:17), MOG34-56 (SEQ ID NO:18), and MOG64-96 (SEQ ID NO:19). The thyroglobulin peptide may comprise an amino acid sequence selected from the group consisting of NIFEXQVDAQPL (SEQ ID NO:155), YSLEHSTDDXASFSRALENATR (SEQ ID NO:156), RALENATRDXFIICPIIDMA (SEQ ID NO:157), LLSLQEPGSKTXSK (SEQ ID NO:158), and EHSTDDXASFSRALEN (SEQ ID NO:159), wherein X is 3,5,3′,5′-tetraiodothyronine (thyroxine). The TPO peptide may comprise an amino acid sequence selected from the group consisting LKKRGILSPAQLLS (SEQ ID NO:160), SGVIARAAEIMETSIQ (SEQ ID NO:161), PPVREVTRHVIQVS (SEQ ID NO:162), PRQQMNGLTSFLDAS (SEQ ID NO:163), LTALHTLWLREHNRL (SEQ ID NO:164), HNRLAAALKALNAHW (SEQ ID NO:165), ARKVVGALHQIITL (SEQ ID NO:166), LPGLWLHQAFFSPWTL (SEQ ID NO:167), MNEELTERLFVLSNSST (SEQ ID NO:168), LDLASINLQRG (SEQ ID NO:169), RSVADKILDLYKHPDN (SEQ ID NO:170), and IDVWLGGLAENFLP (SEQ ID NO:171). The Pendrin peptide may comprise an amino acid sequence selected from the group consisting of QQQHERRKQERK (SEQ ID NO:172) and PTKEIEIQVDWNSE (SEQ ID NO:173). The glucose-6-phosphatase peptide may comprise an amino acid sequence selected from the group consisting of IGRP₁₃₋₂₅ (QHLQKDYRAYYTF) (SEQ ID NO:8), IGRP₂₃₋₃₅ (YTFLNFMSNVGDP) (SEQ ID NO:9), IGRP₂₂₆₋₂₃₈ (RVLNIDLLWSVPI) (SEQ ID NO:10), IGRP₂₄₇₋₂₅₉ (DWIHIDTTPFAGL) (SEQ ID NO:11), G6 Pase-α₂₂₈₋₂₄₀ (KGLGVDLLWTLEK) (SEQ ID NO:12), G6 Pase-α₂₄₉₋₂₆₁ (EWVHIDTTPFASL) (SEQ ID NO:13), UGRP₂₁₈₋₂₃₀ (FTLGLDLSWSISL) (SEQ ID NO:14), and UGRP₂₃₉₋₂₅₁ (EWIHVDSRPFASL) (SEQ ID NO:15). The PLP peptide may comprise an amino acid sequence selected from the group consisting of PLP30-49 (SEQ ID NO:28), PLP40-60 (SEQ ID NO:29), PLP180-199 (SEQ ID NO:30), PLP184-199 (SEQ ID NO:31), and PLP190-209 (SEQ ID NO:32). The MBP peptide may comprise an amino acid sequence selected from the group consisting of MBP66-88 (SEQ ID NO:21), MBP85-99 (SEQ ID NO:22), MBP86-105 (SEQ ID NO:23), MBP143-168 (SEQ ID NO:24), MBP83-97 (SEQ ID NO:25), and MBP85-96 (SEQ ID NO:26). The zona pellucida 3 peptide may comprise an amino acid sequence selected from the group consisting of ZP3 330-342 (NSSSSQFQIHGPR) (SEQ ID NO:42), ZP3 335-342 (QFQIHGPR) (SEQ ID NO:43), and ZP3 330-340 (NSSSSQFQIHG) (SEQ ID NO:44). The human myosin peptide may comprise an α-myosin peptide selected from the group consisting of SLKLMATLFSTYASADTGDSGKGKGGKKKG (amino acids 614-643; where Ac is an acetyl group) (SEQ ID NO:46), GQFIDSGKAGAEKL (amino acids 735-747) (SEQ ID NO:47), and DECSELKKDIDDLE (amino acids 947-960) (SEQ ID NO:48). The Coxsackievirus B4 structural protein peptide is selected from Table 1. The group streptococcal M5 peptide may comprise an amino acid sequence selected from the group consisting of NT4 (GLKTENEGLKTENEGLKTE) (SEQ ID NO:94), NT5 (KKEHEAENDKLKQQRDTL) (SEQ ID NO:95), B1B2 (VKDKIAKEQENKETIGTL) (SEQ ID NO:96), B2 (TIGTLKKILDETVKDKIA) (SEQ ID NO:97), B3A (IGTLKKILDETVKDKLAK) (SEQ ID NO:98), and C3 (KGLRRDLDASREAKKQ) (SEQ ID NO:99), and a peptide selected from Table 2. The peptide may comprise the amino acid sequence (Q/R)(K/R) RAA (SEQ ID NO:190). The type II collagen peptide may comprise an amino acid sequence selected from the group consisting of residues 263-270 (SEQ ID NO:152), 184-198 (SEQ ID NO:153), and 359-369 (SEQ ID NO:154) of type II collagen. The AChR peptide may comprise an amino acid sequence selected from the group consisting of amino acids 37-429, 149-156, 138-167, 149-163, 143-156, 1-181, and 1-437 of human AChR alpha subunit. The Human S-Antigen may comprise an amino acid sequence selected from the group consisting of Peptide 19 (181-VQHAPLEMGPQPRAEATWQF-200) (SEQ ID NO:183), Peptide 35 (341-GFLGELTSSEVATEVPFRLM-356) (SEQ ID NO:184), and Peptide 36 (351-VATEVPFRLMHPQPEDPAKE-370 (SEQ ID NO:185). The DNA may comprise an expression vector capable of expressing the peptide.

The vector may be selected from the group consisting of pVAX, pcDNA3.0, and provax.

Also provided herein are methods of treating type I diabetes mellitus comprising administering to a patient in need thereof the vaccine, wherein the vaccine may comprise the insulin peptide. A method of treating type I diabetes mellitus comprising administering to a patient in need thereof a vaccine, wherein the vaccine may comprise an antigenic insulin peptide and a DNA encoding the insulin peptide, and wherein the peptide has an IC₅₀ of 50 nM or less for MHC Class II. The MHC Class II may be expressed on a tolerogenic dendritic cell. The peptide may consist of the amino acid sequence MRLLPLLALLA (SEQ ID NO:5) or SHLVEALYLVCGERG (SEQ ID NO:191).

Further provided herein are methods of treating multiple sclerosis comprising administering to a patient in need thereof the vaccine, wherein the vaccine may comprise a multiple sclerosis autoantigenic peptide and a DNA encoding the peptide, and wherein the peptide has an IC₅₀ of 50 nM or less for MHC Class II. The vaccine may comprise the myelin oligodendrocyte glycoprotein (MOG), the myelin basic protein (MBP), the proteolipid protein (PLP), the myelin-associated oligodendrocyte basic protein (MOBP), or the oligodendrocyte-specific protein (OSP); and the peptide is a peptide of MOG. Also, the peptide may consist of an amino acid sequence selected from the group consisting of HPIRALVGDEVELP, VGWYRPPFSRVVHLYRNGKD, and LKVEDPFYWVSPGVLVLLAVLPVLLL.

Also provided herein are methods of treating autoimmune ovarian disease comprising administering to a patient in need thereof the vaccine, wherein the vaccine may comprise the zonapellucida protein peptide. Further provided herein are methods of treating a house dust mite allergy comprising administering to a patient in need thereof the vaccine, wherein the vaccine may comprise the antigenic Dermatophagoides pteronyssinus 1 peptide.

Also provided herein are methods for treating asthma comprising administering to a patient in need thereof the vaccine, wherein the vaccine comprises Der-p1, ovalbumin, or other allergen.

Further provided herein are methods of treating myocarditis comprising administering to a patient in need thereof the vaccine, wherein the vaccine may comprise the α-myosin peptide, the Coxsackievirus B4 structural protein peptide, or the group A streptococcal M5 protein peptide. Also provided herein are methods of treating rheumatoid arthritis comprising administering to a patient in need thereof the vaccine, wherein the vaccine may comprise the peptide (Q/R)(K/R) RAA (SEQ ID NO:190), or the type II collagen peptide. Further provided herein are methods of treating thyroiditis comprising administering to a patient in need thereof the vaccine, wherein the vaccine may comprise the thyroid peroxidase (TPO), thyroglobulin, or Pendrin peptide. Also provided herein is a method of treating myasthenia gravis comprising administering to a patient in need thereof the vaccine, wherein the vaccine may comprise the acetylcholine receptor peptide. Further provided herein are methods of treating autoimmune uveitis comprising administering to a patient in need thereof the vaccine, wherein the vaccine may comprise the human S-antigen peptide.

Also provided herein are methods of treating a house dust mite allergy comprising administering to a patient in need thereof a vaccine, wherein the vaccine may comprise an antigenic Dermatophagoides pteronyssinus 1 peptide and a DNA encoding the peptide, and wherein the peptide has an IC₅₀ of 50 nM or less for MHC Class II.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

A “peptide” or “polypeptide” is a linked sequence of amino acids and can be natural, synthetic, or a modification or combination of natural and synthetic.

“Treatment” or “treating,” when referring to protection of an animal from a disease, means preventing, suppressing, repressing, or completely eliminating the disease. Preventing the disease involves administering a composition of the present invention to an animal prior to onset of the disease. Suppressing the disease involves administering a composition of the present invention to an animal after induction of the disease but before its clinical appearance. Repressing the disease involves administering a composition of the present invention to an animal after clinical appearance of the disease.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids.

A “variant” can mean means a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in peptides retaining biological activity, for example immunogenicity, as is understood in the art. Substitutions can be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

### 2. Vaccine

Provided herein are vaccines that are comprised of an antigen and a DNA encoding the same antigen. The vaccine can induce antigen-specific iTreg cells that inhibit antigen-specific T cell function. The combination of an antigen and DNA encoding the antigen in the vaccine induces iTreg cells efficiently against specific antigens far better than either a vaccine comprising an antigen or its corresponding DNA alone. The vaccine further enhances MHC Class II presentation and expression for iTreg cell induction.

a. Antigen

The vaccine may comprise autoimmune disease antigens or variants thereof, and DNA encoding the same. The antigen can be an autologous antigen, and can induce antigen-specific iTreg cells that inhibit antigen-specific T cell function. Co-immunization with sequence-matched DNA and protein antigens induce regulatory DCs (DCregs) of a CD11c⁺CD40^{low}IL-10⁺ phenotype in vitro and in vivo, which in turn mediates antigen-specific tolerance.

Conventional DCs (DCs) are specialized antigen-presenting cells (APCs) that can be broadly callified into the CD11c⁺CD8a⁺ and CD11c⁺CD8a⁻ subtypes, both of which have a remarkable functional plasticity in the induction of immunity or tolerance, depending on their maturation status. Immature DCs (iDCs) can promote tolerance by converting naïve T cells into the CD4⁺Foxp3⁺ regulatory T cells (Tregs). Signals from the DNA construct and the sequence matched protein of the vaccine can act in a concerted manner to activate regulatory signals that convert normal DCs into DCregs.

DNA and protein antigen co-immunization induces DCregs by allowing co-uptake of the DNA and protein immunogens by the same DC primarily via caveolae-mediated endocytosis. This event down-regulates the phosphorylation of Cav-1 and up-regulates Tollip, which in turn initiates downstream signaling that up-regulates SOCS1 and down-regulates NF-κB and STAT-1α. The down-regulation of NF-κB explains the CD40low and IL-10+ phenotype of the co-immunization-induced DCregs. DCregs may be generated in vitro in both primary DCs and DC lines by feeding them with DNA and protein immunogen for as short as 24 h. The in vitro generated DCregs are effective for treating inflammatory and autoimmune diseases, presumably by inducing antigen-specific CD25− iTreg.

Cav-1 is the key protein to form caveolae. It also regulates signal transduction through compartmentalization of numerous signaling molecules. Cav-1, Tollip and IRAK-1 form a complex to suppress the IRAK-1's kinase activity during resting conditions. Cav-1 dissociates from the complex once phosphorylated, which leads to phosphorylation of IRAK-1 in the cytosol and activation of the downstream signaling cascade, including translocation of NF-κB25. Co-uptake of DNA and protein down-regulates phosphorylation of Cav-1, thereby preventing the activation NF-κB. Accordingly, a DNA antigen and a sequence-matched protein antigen can convert normal DCs into DCregs. The same DC is required for acquisition of the DCreg phenotype and function and that the co-uptake event triggers Cav-1 and Tollip co-dependent signaling that up-regulates SOCS1 and down-regulates NF-κB and STAT-1α.

Tollip plays an important role in inhibition of innate responses and maintenance of a resting state. Co-uptake of DNA and protein antigens upregulates Tollip and silencing of Tollip, or Cav-1, partially blocks the differentiation of DCs into DCregs, suggesting that Tollip may act independently of Cav-1 in down-regulating NF-κB.

The iTreg cells can be CD4⁺CD25⁺ and also exhibit high expression of Foxp3. The iTreg cells can be capable of specific prevention of and interference with unwanted immunity in the absence of general immunosuppression. Proliferation of the iTreg cells can be induced by high doses of interleukin 2 (IL-2). The iTreg cells can be capable of suppressing effector T cells by virtue of the presence of CD80 and CD86 ligands on activated CD4⁺ effector T cells. Once the iTreg cells are activated by a T cell receptor ligand, the presence of an antigen presenting cell can or cannot be necessary in the suppression of effector T cells. After antigenic stimulation, the iTreg cells can home to antigen-draining lymph nodes and can accumulate through cell division at the same rate as naïve T cells.

Production of the iTreg cells can require MHC Class II expression on cortical epithelial cells. The receptors can be MHC restricted, and the iTreg cells can be specific for the antigen. It can be possible via an IL-10-based mechanism to induce the iTreg cells to participate in bystander-mediated regulation. iTreg cells cause a reduction in inflammatory T_{Helper} and T_{Killer} cells. The iTreg suppression may occur by interaction with the antigen-presenting cells, including DCs and epithelial cells, for example in the lung or other organ, where the antigen specific iTreg cells are retained by reducing their expression of the egress molecule S1P1. The interaction upregulates expression of chemoattracting IP-10 of antigen specific APCs, which trap the CXCR3⁺ inflammatory T cells into epithelial cells (i.e. T_{H1}, T_{K1}, etc.). Twenty percent of these trapped T cells undergo apoptosis and a few are then converted into IL10 and TGF-beta expressing Treg cells. Therefore, the inflammatory T cells are reduced in organs, like the lungs, and conditions, such as asthma, are ameliorated.

The antigen can be associated with allergy, asthma, or an autoimmune disease. The antigen can affect a mammal, which can be a human, chimpanzee, dog, cat, horse, cow, mouse, or rat. The antigen can be contained in a protein from a mammal, which can be a human, chimpanzee, dog, cat, horse, cow, pig, sheep, mouse, or rat.

(1) FSA1

The antigen can be a peptide of the flea allergen FSA1, or a variant thereof, which can have amino acids 66-80 or amino acids 100-114 of FSA1.

(2) Der-p1

The antigen can also be a peptide of Der-p1, or a variant thereof. The Der-p1 can have the sequence of GeneBank Access No. EU092644, the contents of which are incorporated herein by reference. This antigen may be related to asthma and/or an allergy.

(3) Type 1 Diabetes Mellitus

The antigen can be an autoantigen involved in type 1 diabetes mellitus, or a variant thereof. The antigen can be a peptide of insulin, and can be proinsulin. The proinsulin antigen can have the sequence MALWMRLLPLLALLALWGPDPAAAFVNQHLCGSHLVEALYLVC GERGFFYTPKTRREAEDLQVGQVELGGGPGAGSLQPLALEGSLQKRGIVEQCCTSICS LYQLENYCN (SEQ ID NO:192), which can be encoded by a sequence contained in GenBank Accession No. NM_{—}000207, the contents of which are incorporated by reference herein. The antigen can be human B9-23. The insulin antigen can also have the sequence MRLLPLLALLA (SEQ ID NO:5), SHLVEALYLVCGERG (SEQ ID NO:191), or LYLVCGERG (SEQ ID NO:6). The antigen can also be a insulin antigen disclosed in Wong S F, TRENDS in Molecular Medicine, 2005; 11(10), the contents of which are incorporated herein by reference. The insulin antigen can have the amino acid sequence GIVEQCCTSICSLYQ (SEQ ID NO:7).

The antigen can be a sequence of a glucose-6-phosphatase (G6P), as described in The Journal of Immunology, 2006; 176:2781-9, the contents of which are incorporated herein by reference. The G6P antigen can have the sequence of IGRP₁₃₋₂₅ (QHLQKDYRAYYTF) (SEQ ID NO:8), IGRP₂₃₋₃₅ (YTFLNFMSNVGDP) (SEQ ID NO:9), IGRP₂₂₆₋₂₃₈ (RVLNIDLLWSVPI) (SEQ ID NO:10), IGRP₂₄₇₋₂₅₉ (DWIHIDTTPFAGL) (SEQ ID NO; 11), G6 Pase-α₂₂₈₋₂₄₀ (KGLGVDLLWTLEK) (SEQ ID NO:12), G6 Pase-α₂₄₉₋₂₆₁ (EWVHIDTTPFASL) (SEQ ID NO:13), UGRP₂₁₈₋₂₃₀ (FTLGLDLSWSISL (SEQ ID NO:14), and UGRP₂₃₉₋₂₅₁ (EWIHVDSRPFASL) (SEQ ID NO:15).

The antigen can also be a peptide of glutamic acid decarboxylase or heat shock protein.

(4) Multiple Sclerosis

The antigen can be an autoantigen involved in multiple sclerosis (MS). The antigen can be a peptide of myelin oligodendrocyte glycoprotein (MOG), myelin basic protein (MBP), proteolipid protein (PLP), myelin-associated oligodendrocyte basic protein (MOBP), or oligodendrocyte-specific protein (OSP), or a variant thereof. The MBP antigen can be MBP66-88 (SEQ ID NO:21), MBP85-99 (SEQ ID NO:22), MBP86-105 (SEQ ID NO:23), MBP143-168 (SEQ ID NO:24), MBP83-97 (SEQ ID NO:25), or MBP85-96 (SEQ ID NO:26). The PLP antigen can be PLP30-49 (SEQ ID NO:28), PLP40-60 (SEQ ID NO:29), PLP180-199 (SEQ ID NO:30), PLP184-199 (SEQ ID NO:31), or PLP190-209 (SEQ ID NO:32). The MOG antigen can be MOG1-22 (SEQ ID NO:17), MOG34-56 (SEQ ID NO:18), or MOG64-96 (SEQ ID NO:19). The MOG antigen can also have the sequence HPIRALVGDEVELP (SEQ ID NO:36), VGWYRPPFSRVVHLYRNGKD (SEQ ID NO:37), or LKVEDPFYWVSPGVLVLLAVLPVLLL (SEQ ID NO:38). The MS antigen can also have a sequence described in Schmidt S, Mult Scler., 1999; 5(3):147-60, the contents of which are incorporated herein by reference.

(5) Autoimmune Ovarian Disease

The antigen can be an autoantigen involved in autoimmune ovarian disease. The antigen can be a peptide contained in zonapellucida (ZP) 1, 2 or 3. The ZP peptide can have the sequence of NCBI Reference Sequences NP_{—}003451.1 (SEQ ID NO:39), NP 009086.4 (SEQ ID NO:40), or NP 997224.2 (SEQ ID NO:41). The ZP antigen can a ZP3 peptide having the sequence ZP3 330-342 (NSSSSQFQIHGPR) (SEQ ID NO:42), ZP3 335-342 (QFQIHGPR) (SEQ ID NO:43), or ZP3 330-340 (NSSSSQFQIHG) (SEQ ID NO:44). The ZP antigen can be a peptide disclosed in Lou Y, The Journal of Immunology, 2000; 164:5251-7, the contents of which are incorporated herein by reference.

(6) Myocarditis

The antigen can be an autoantigen involved in myocarditis. The antigen can be a peptide described in Smith S C, Journal of Immunology, 1991; 147(7):2141-7, the contents of which are incorporated herein by reference. The antigen can be a peptide contained in human myosin, which can have the sequence of GeneBank Accession No. CAA86293.1 (SEQ ID NO:45). The antigen can be a peptide contained within α-myosin, and can have the sequence Ac-SLKLMATLFSTYASADTGDSGKGKGGKKKG (amino acids 614-643; where Ac is an acetyl group) (SEQ ID NO:46), GQFIDSGKAGAEKL (amino acids 735-747) (SEQ ID NO:47), or DECSELKKDIDDLE (amino acids 947-960) (SEQ ID NO:48), as disclosed in Pummerer, C L, J. Clin. Invest. 1996; 97:2057-62, the contents of which are incorporated herein by reference. The antigen can also be a Coxsackievirus B4 structural protein peptide having one of the following sequences.

TABLE 1

| Coxsackievirus | | | |
|---|---|---|---|
| B4 | | | SEQ |
| Structural | Amino | | ID |
| Protein | Acids | Sequence | NO. |
| VP4 | 1-20 | MGAQVSTQKTGAHETSLSAS | 49 |
| VP4 | 21-40 | GNSIIHYTNINYYKDAASNS | 50 |
| VP4 | 31-50 | NYYKDAASNSANRQDFTQDP | 51 |
| VP4 | 41-60 | ANRQDFTQDPSKFTEPVKDV | 52 |
| VP4 | 51-70 | SKFTEPVKDVMIKSLPALNS | 53 |
| VP2 | 61-80 | MIKSLPALNSPTVEECGYSD | 54 |
| VP2 | 71-90 | PTVEECGYSDRVRSITLGNS | 55 |
| VP2 | 81-100 | RVRSITLGNSTITTQECANV | 56 |
| VP2 | 91-110 | TITTQECANVVVGYGVWPDY | 57 |
| VP2 | 111-130 | LSDEEATAEDQPTQPDVATC | 58 |
| VP2 | 121-140 | QPTQPDVATCRFYTLNSVKW | 59 |
| VP2 | 131-150 | RFYTLNSVKWEMQSAGWWWK | 60 |
| VP2 | 151-170 | FPDALSEMGLFGQNMQYHYL | 61 |
| VP2 | 161-180 | FGQNMQYHYLGRSGYTIHVQ | 62 |
| VP2 | 171-190 | GRSGYTIHVQCNASKFHQGC | 63 |
| VP2 | 181-200 | CNASKFHQGCLLVVCVPEAE | 64 |
| VP2 | 211-230 | AYGDLCGGETAKSFEQNAAT | 65 |
| VP2 | 221-240 | AKSFEQNAATGKTAVQTAVC | 66 |
| VP2 | 231-250 | GKTAVQTAVCNAGMGVGVGN | 67 |
| VP2 | 251-270 | LTIYPHQWINLRTNNSATIV | 68 |
| VP2 | 261-280 | LRTNNSATIVMPYINSVPMD | 69 |
| VP2 | 271-290 | MPYINSVPMDNMFRHNNFTL | 70 |
| VP2 | 281-300 | NMFRHNNFTLMIIPFAPLDY | 71 |
| VP3 | 321-340 | YNGLRLAGHQGLPTMLTPGS | 72 |
| VP3 | 351-370 | SPSAMPQFDVTPEMNIPGQV | 73 |
| VP3 | 361-380 | TPEMNIPGQVRNLMEIAEVD | 74 |
| VP3 | 371-390 | RNLMEIAEVDSVVPINNLKA | 75 |
| VP3 | 381-400 | SVVPINNLKANLMTMEAYRV | 76 |
| VP3 | 391-410 | NLMTMEAYRVQVRSTDEMGG | 77 |
| VP3 | 401-420 | QVRSTDEMGGQIFGFPLQPG | 78 |
| VP3 | 411-430 | QIFGFPLQPGASSVLQRTLL | 79 |
| VP3 | 421-440 | ASSVLQRTLLGEILNYYTHW | 80 |
| VP3 | 431-450 | GEILNYYTHWSGSLKLTFVF | 81 |
| VP3 | 441-460 | SGSLKLTFVFCGSAMATGKF | 82 |
| VP3 | 511-530 | DDKYTASGFISCWYQTNVIV | 83 |
| VP3 | 541-560 | MCFVSACNDFSVRMLRDTQF | 84 |
| VP1 | 671-690 | LRRKMEMFTYIRCDMELTFV | 85 |
| VP1 | 721-740 | VPTSVNDYVWQTSTNPSIFW | 86 |
| VP1 | 731-750 | QTSTNPSIFWTEGNAPPRMS | 87 |
| VP1 | 741-760 | TEGNAPPRMSIPFMSIGNAY | 88 |
| VP1 | 751-770 | IPFMSIGNAYTMFYDGWSNF | 89 |
| VP1 | 771-790 | SRDGIYGYNSLNNMGTIYAR | 90 |
| VP1 | 781-800 | LNNMGTIYARHVNDSSPGGL | 91 |
| VP1 | 791-810 | HVNDSSPGGLTSTIRIYFKP | 92 |
| VP1 | 831-850 | SVNFDVEAVTAERASLITTG | 93 |

The antigen can be a peptide contained in a Coxsackie virus B4 structural protein as disclosed in Marttila J, Virology, 2000; 293:217-24, the contents of which are incorporated herein by reference in its entirety.

The antigen can also be a peptide from group A streptococcal M5 protein. The M5 peptide can have one of the following sequences: NT4 (GLKTENEGLKTENEGLKTE) (SEQ ID NO:94), NT5 (KKEHEAENDKLKQQRDTL) (SEQ ID NO:95), B1B2 (VKDKIAKEQENKETIGTL) (SEQ ID NO:96), B2 (TIGTLKKILDETVKDKIA) (SEQ ID NO:97), B3A (IGTLKKILDETVKDKLAK) (SEQ ID NO:98), and C3 (KGLRRDLDASREAKKQ) (SEQ ID NO:99). The antigen can also be a M5 peptide from the following table.

TABLE 2

| M5 epitope position | Sequence | SEQ ID NO. |
|---|---|---|
| 27-44 | LKTKNEGLKTENEGLKTE | 100 |
| 59-76 (NT5) | KKEHEAENDKLKQQRDTL | 101 |
| 72-89 (NT6) | QRDTLSTQKETLEREVQN | 102 |
| 85-102 (NT7) | REVQNTQYNNETLKIKNG | 103 |
| 98-115 (NT8) | KIKNGDLTKELNKTRQEL | 104 |
| 111-129 (B1A) | TRQELANKQQESKENEKAL | 105 |
| 150-167 (B2) | TIGTLKKILDETVKDKIA | 106 |
| 176-193 (B3A) | IGTLKKILDETVKDKLAK | 107 |
| 1-35 | AVTRGTINDPQRAKEALDKYELENHDLKTKNEGLK | 108 |
| 28-54 | KTKNEGLKTENEGLKTENEGLKTENEG | 109 |
| 55-70 | LKTEKKEHEAENDKLK | 110 |
| 103-132 | DLTKELNKTROELANKQQESKENEKAINEL | 111 |
| 133-162 | LEKTVKDKIAKEQENKETIGTLKKILDETV | 112 |
| 209-223 | TIGTLKKILDETVKDK | 113 |
| 217-237 | ISDASRKGLRRDLDASREAKK | 114 |
| 300-319 | DASREAKKQVEKAIEEANSK | 115 |
| 312-331 | ALEEANSKLAALEKLNKELE | 116 |
| 329-359 | ELEESKKLTEKEKAELQAKLEAEAKQLKEQL | 117 |
| 359-388 | AKQAEELAKLRAGKASDSQTPDTKPGNKAV | 118 |
| 389-425 | VPGKGQAPQAGTKPNQNKAPMKETKRQLPSTGETANP | 119 |
| 295-313 | LRRDLDASREAKKQVEKAI | 120 |
| 305-324 | AKKQVEKALEEANSKLAALE | 121 |
| 335-354 | KLTEKEKAELQAKLEAEAKA | 122 |
| 345-364 | QAKLEAEAKALKEQLAKQAE | 123 |
| 355-374 | LKEQLAKQAEELAKLRAGKA | 124 |
| 1-25 | TVTRGTISDPQRAKEALDKYELENH | 125 |
| 81-96 | DKLKQQRDTLSTQKETLEREVQNI | 126 |
| 163-177 | ETIGTLKKILDETVK | 127 |
| 1-18 | AVTRGTINDPQRAKEALD | 128 |
| 14-31 | KEALDKYELENHDLKTKN | 129 |
| 27-44 | LKTKNEGLKTENEGLKTE | 130 |
| 40-58 | GLKTENEGLKTENEGLKTE | 131 |
| 59-76 | KKEHEAENDKLKQQRDTL | 132 |
| 72-89 | QRDTLSTQKETLEREVQN | 133 |
| 85-102 | REVQNTQYNNETLKIKNG | 134 |
| 98-115 | KIKNGDLTKELNKTRQEL | 135 |
| 111-129 | TRQELANKQQESKENEKAL | 136 |
| 124-141 | ENEKALNELLEKTVKDKI | 137 |
| 137-154 | VKDKIAKEQENKETIGTL | 138 |
| 150-167 | TIGTLKKILDETVKDKIA | 139 |
| 163-180 | KDKIAKEQENKETIGTLK | 140 |
| 176-193 | IGTLKKILDETVKDKLAK | 141 |
| 189-206 | DKLAKEQKSKQNIGALKQ | 142 |
| 202-219 | GALKQELAKKDEANKISD | 143 |
| 215-232 | NKISDASRKGLRRDLDAS | 144 |
| 228-245 | DLDASREAKKQLEAEHQK | 145 |
| 241-258 | AEHQKLEEQNKISEASRK | 146 |
| 254-271 | EASRKGLRRDLDASREAK | 147 |
| 267-284 | SREAKKQLEAEQQKLEEQ | 148 |
| 280-297 | KLEEQNKISEASRKGLRR | 149 |
| 293-308 | KGLRRDLDASREAKKQ | 150 |

The peptide can also be a sequence disclosed in Cunningham M W, INFECTION AND IMMUNITY, 1997; 65(9):3913-23, the contents of which are incorporated herein by reference in its entirety.

(7) Rheumatoid Arthritis

The antigen can be an autoantigen involved in rheumatoid arthritis (RA). The antigen can be a peptide having the sequence Q/R, K/R, R, A, and A, described in Fox D A, Arthritis and Rheumatism, 1997; 40(4):598-609, Mackay I R, J Rheumatol, 2008; 35; 731-733, or Hill J A, The Journal of Immunology, 2003; 171:538-41, the contents of which are incorporated herein by reference in their entirety. The antigen can be a peptide of type II collagen, which can have the sequence of amino acids 263-270 (SEQ ID NO:152) or 184-198 (SEQ ID NO:153) of type II collagen. The type II collagen antigen can be a peptide disclosed in Staines N A, Clin. Exp. Immunol., 1996; 103:368-75 or Backlund J, PNAS, 2002; 99(15):9960-5, the contents of which are incorporated herein by reference in their entirety. The type II collagen antigen can also have the sequence of amino acid residues 359-369 [C1^{III}] (SEQ ID NO:154) of type II collagen, as disclosed in Burkhardt, H, ARTHRITIS & RHEUMATISM, 2002; 46(9):2339-48, the contents of which are incorporated herein by reference in its entirety.

(8) Thyroiditis

The antigen can be an autoantigen involved in thyroiditis, and can be a peptide contained in thyroid peroxidase (TPO), thyroglobulin, or Pendrin. The antigen can be described in Daw K, Springer Seminlmmunopathol, 1993, 14:285-307; “Autoantigens in autoimmune thyroid diseases, The Japanese Journal of Clinical Pathology, 1989; 37(8): 868-74; Fukuma N, Clin. Exp. Immunol., 1990; 82(2):275-83; or Yoshida A, The Journal of Clinical Endocrinology & Metabolism, 2009; 94(2):442-8, the contents of which are incorporated herein by reference in their entirety.

The thyroglobulin antigen can have the sequence, NIFEXQVDAQPL (SEQ ID NO:155), YSLEHSTDDXASFSRALENATR (SEQ ID NO:156), RALENATRDXFIICPIIDMA (SEQ ID NO:157), LLSLQEPGSKTXSK (SEQ ID NO:158), or EHSTDDXASFSRALEN (SEQ ID NO:159), wherein X is 3,5,3′,5′-tetraiodothyronine (thyroxine). The TPO antigen can have the sequence LKKRGILSPAQLLS (SEQ ID NO:160), SGVIARAAEIMETSIQ (SEQ ID NO:161), PPVREVTRHVIQVS (SEQ ID NO:162), PRQQMNGLTSFLDAS (SEQ ID NO:163), LTALHTLWLREHNRL (SEQ ID NO:164), HNRLAAALKALNAHW (SEQ ID NO; 165), ARKVVGALHQIITL (SEQ ID NO:166), LPGLWLHQAFFSPWTL (SEQ ID NO:167), MNEELTERLFVLSNSST (SEQ ID NO:168), LDLASINLQRG (SEQ ID NO:169), RSVADKILDLYKHPDN (SEQ ID NO:170), or IDVWLGGLAENFLP (SEQ ID NO:171). The Pendrin antigen can have the sequence QQQHERRKQERK [amino acids 34-44 in human pendrin (GenBank AF030880)] (SEQ ID NO:172), PTKEIEIQVDWNSE [amino acids 630-643 in human pendrin] (SEQ ID NO:173), or NCBI GenBank Accession No. NP_{—}000432.1 (SEQ ID NO:174).

(9) Myasthenia Gravis

The antigen can be an autoantigen involved in myasthenia gravis (MG), and can be contained in acetylcholine receptor (AChR). The antigen can be a peptide described in Protti M A, Immunology Today, 1993; 14(7):363-8; Hawke S, Immunology Today, 1996; 17(7):307-11, the contents of which are incorporated herein by reference. The AChR antigen can be amino acids 37-429 (SEQ ID NO:176), 149-156 (SEQ ID NO:177), 138-167 (SEQ ID NO:178), 149-163 (SEQ ID NO:179), 143-156 (SEQ ID NO:180), 1-181 (SEQ ID NO:181), or 1-437 (SEQ ID NO:182) of human AChR alpha subunit.

(10) Autoimmune Uveitis

The antigen can be an autoantigen involved in autoimmune uveitis (AU), and can be contained in Human S-Antigen. The antigen can have the sequence of Peptide 19 (181-VQHAPLEMGPQPRAEATWQF-200) (SEQ ID NO:183), Peptide 35 (341-GFLGELTSSEVATEVPFRLM-356) (SEQ ID NO:184), or Peptide 36 (351-VATEVPFRLMHPQPEDPAKE-370) (SEQ ID NO:185). The antigen can be described in de Smet M D, J. Autoimmun. 1993; 6(5):587-99, the contents of which are incorporated herein by reference. The antigen can also be contained in Human IRBP, and can have the sequence 521-YLLTSHRTATAAEEFAFLMQ-540 (SEQ ID NO:186). The antigen can be described in Donoso L A, J. Immunol., 1989; 143(1):79-83, the contents of which are incorporated herein by reference in its entirety.

(11) Other Antigens

The antigen can also be an antigen as disclosed in U.S. Patent Application Publication No. 20100143401, the contents of which are incorporated herein by reference in its entirety.

(12) MHC Class II Binding Affinity

The antigen can have a high affinity for MHC Class II (MHC-II), which can increase induction of iTreg cells. The MHC-II affinity of the antigen can be an IC₅₀ of less than or equal to 50 nM. The affinity can also be an IC₅₀ of less than or equal to 100, 95, 90, 85, 80, 75, 70, 65, 60, 55, 50, 45, 40, 35, 30, 25, 20, 15, 10, 9, 8, 7, 6, 4, 3, 2, 1, 0.9, 0.8, 0.7, 0.6, 0.5, 0.4, 0.3, 0.2, or 0.1 nM.

The affinity of the antigen for MCH-II can be predicted using a computer algorithm. The algorithm can be MHCPred, as described by Guan P, Doytchinova I A, Zygouri C, Flower D R, MHCPred: bringing a quantitative dimension to the online prediction of MHC binding, Appl Bioinformatics. 2003 2:63-66; Guan P, Doytchinova I A, Zygouri C, Flower D R, MHCPred: A server for quantitative prediction of peptide-MHC binding, Nucleic Acids Res. 2003 31:3621-3624; and Hattotuwagama C K, Guan P, Doytchinova I A, Zygouri C, Flower D R, Quantitative online prediction of peptide binding to the major histocompatibility complex, J Mol Graph Model. 2004 22:195-207, the contents of which are incorporated herein by reference in their entirety. The algorithm can also be NN-align or SMM-align, as described by Nielsen M and Lund O, NN-align, a neural network-based alignment algorithm for MHC class II peptide binding prediction, BMC Bioinformatics. 2009; 10:296; and Nielsen M, Lundegaard C, Lund O, Prediction of MHC class II binding affinity using SMM-align, or a novel stabilization matrix alignment method, BMC Bioinformatics. 2007; 8:238, the contents of which are incorporated herein by reference in their entirety.

b. DNA

Also provided herein are DNA that encode the antigen. The DNA can include an encoding sequence that encodes the antigen. The DNA can also include additional sequences that encode linker or tag sequences that are linked to the antigen by a peptide bond.

c. Vector

Further provided herein are vectors that include the DNA. The vector can be capable of expressing the antigen. The vector may be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

i. Expression Vectors

The vector may be circular plasmid or a linear nucleic acid vaccine. The circular plasmid and linear nucleic acid are capable of directing expression of a particular nucleotide sequence in an appropriate subject cell. The vector may have a promoter operably linked to the antigen-encoding nucleotide sequence, which may be operably linked to termination signals. The vector may also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

ii. Circular and Linear Vectors

The vector may be circular plasmid, which may transform a target cell by integration into the cellular genome or exist extrachromosomally (e.g. autonomous replicating plasmid with an origin of replication).

The vector can be pVAX, pcDNA3.0, or provax, or any other expression vector capable of expressing the DNA and enabling a cell to translate the sequence to a antigen that is recognized by the immune system. The vector can be combined with antigen at a mass ratio of between 5:1 and 1:5, or of between 1:1 and 2:1.

Also provided herein is a linear nucleic acid vaccine, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, a polyadenylation signal. The expression of the antigen may be controlled by the promoter. The LEC may not contain any antibiotic resistance genes and/or a phosphate backbone. The LEC may not contain other nucleic acid sequences unrelated to the desired antigen gene expression.

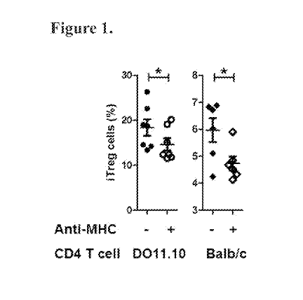

The LEC may be derived from any plasmid capable of being linearized. The plasmid may be capable of expressing the antigen. The plasmid may be pNP (Puerto Rico/34) or pM2 (New Calcdonia/99). See FIG. 1. The plasmid may be pVAX, pcDNA3.0, or provax, or any other expression vector capable of expressing the DNA and enabling a cell to translate the sequence to a antigen that is recognized by the immune system.

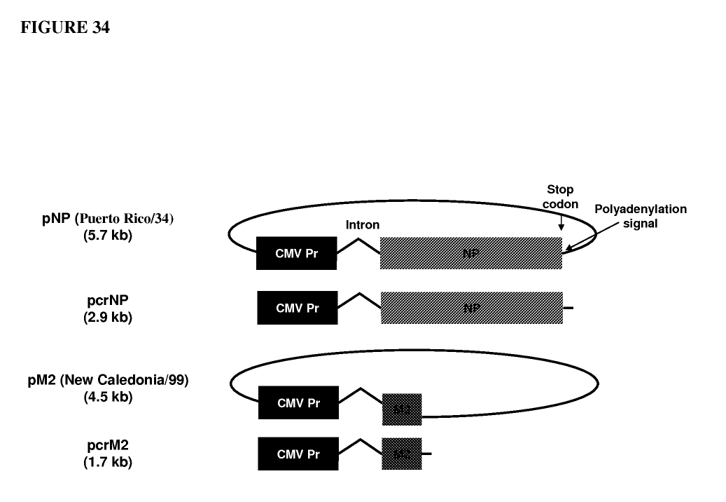

The LEC may be perM2. The LEC may be perNP. perNP and perMR may be derived from pNP (Puerto Rico/34) and pM2 (New Calcdonia/99), respectively. See FIG. 34. The LEC may be combined with antigen at a mass ratio of between 5:1 and 1:5, or of between 1:1 to 2:1.

iii. Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleic acid sequence encoding the antigen and signals required for efficient polyadenylation of the transcript, ribosome binding sites, and translation termination. The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

d. Other Components of Vaccine-Adjuvants, Excipients

The vaccine may further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate is may be present in the vaccine at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. The DNA plasmid vaccines may also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in alternative plasmid or are delivered as proteins in combination with the plasmid above in the vaccine. The adjuvant may be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine may further comprise a genetic vaccine facilitator agent as described in U.S. Serial No. 021,579 filed Apr. 1, 1994, which is fully incorporated by reference.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

### 3. Method of Vaccination

Provided herein are methods of vaccinating a patient to treat or prevent a symptom of allergy, asthma, an autoimmune disease, or transplant rejection using the vaccine. The allergy can be flea allergic dermatitis or a house dust mite allergy. The autoimmune disease can be type I diabetes mellitus, multiple sclerosis, autoimmune ovarian disease, myocarditis, rheumatoid arthritis, asthma, thyroiditis, myasthenia gravis, or autoimmune uveitis.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

a. Administration

The vaccine can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The vaccine can be administered prophylactically or therapeutically. In prophylactic administration, the vaccines can be administered in an amount sufficient to induce iTreg responses. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Feigner et al. (U.S. Pat. No. 5,580,859, issued Dec. 3, 1996); Feigner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccines can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be incorporated into liposomes, microspheres or other polymer matrices (Felgner et al., U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the vaccine described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the vaccine into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. No. 6,520,950; U.S. Pat. No. 7,171,264; U.S. Pat. No. 6,208,893; U.S. Pat. No. 6,009,347; U.S. Pat. No. 6,120,493; U.S. Pat. No. 7,245,963; U.S. Pat. No. 7,328,064; and U.S. Pat. No. 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA (Inovio Pharmaceuticals, Blue Bell Pa.) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so user's have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

The present invention has multiple aspects, illustrated by the following non-limiting examples.

### Example 1

### Treating Dermatitis Using a Combined Peptide/DNA Vaccine

This example demonstrates the characteristics of highly antigenic epitopes for CD25⁻ iTreg, including the ability to block induction of CD25⁻ iTreg by tolerogenic DC by using anti-MHC-II antibody. Further, both the number and the suppressive activity of CD25⁻ iTreg correlates positively with the overt antigenicity of an epitope to active T cells. Finally, in a mouse model of dermatitis, highly antigenic epitopes derived from a flea allergen not only induced more CD25⁻ iTreg, but also more effectively prevented allergenic reaction to the allergen than did weakly antigenic epitopes. Together, efficient induction of CD25⁻ iTreg requires highly antigenic peptide epitopes. These results demonstrate that highly antigenic epitopes, with higher affinities for MHC-II should be used for efficient induction of iTreg cells for clinical applications.

The inducible regulatory T cells, or iTreg, differ from the naturally regulatory T cells (nTreg) in that the former are generated in the periphery through encounter with environmental antigens. It is also believed that iTreg play non-overlapping roles, relative to nTreg, in regulating peripheral tolerance. Most iTreg reported to date have been CD25⁺ cells (CD4⁺CD25⁺Foxp3⁺), and it is well established that their induction requires suboptimal stimulation of the T cell receptor (TCR) and cytokines TGF-β and IL-2. The CD25⁺ iTreg thus appear to derive primarily from weakly stimulated CD4⁺ T cells.

A different subset of iTreg that is CD25⁻ (CD4⁺CD25⁻Foxp3⁺) have been identified. The CD25⁻ iTreg are induced after co-immunization using a protein antigen and a DNA vaccine encoding the same antigen. Unlike that of the CD25⁺ iTreg, the induction of the CD25− iTreg involves the generation of CD40^{low}IL-10^{high} tolerogenic dendritic cells (DCs), which in turn mediate the induction of CD25⁻ iTreg in an antigen-specific manner. In mouse models, this subset of iTreg is potentially useful as a therapeutic for allergic and autoimmune diseases, such as asthma, flea allergic dermatitis (FAD), and type 1 diabetes (T1D).

While the requirement for weak antigen stimulation is well established for the induction of CD25⁺ iTreg, it is unclear whether the same is true for the induction of CD25⁻ iTreg. Addressing this question will allow not only to further differentiation of the two subsets of iTreg, but also maximization of the tolerogenicity of co-immunization by choosing T cell epitopes of appropriate antigenicity.

### Example 2

### MHC-Ag:TCR Interaction is Required for Induction of CD25⁻ iTreg

To test whether the MHC-Ag:TCR interaction is required for the induction of CD25⁻ iTreg, an in vitro iTreg induction system was employed. It involved culture of CD4⁺ T cells together with co-immunization-induced tolerogenic DCs that present the dominant epitope of hen ovalbumin, OVA₃₂₃₋₃₃₉ (SEQ ID NO:187). Using either clonotypic CD4⁺ T cells from DO11.10 Balb/c mice or polyclonal CD4⁺ T cells from ovalbumin-sensitized Balb/c mice, it was found that the induction of CD25⁻ iTreg in either case could be blocked by anti-MHC-II antibody and, therefore, was MHC-II-dependent. Thus, antigenic stimulation is essential for the induction of CD25⁻ iTreg (FIG. 1).

### Example 3

### Highly Antigenic Epitopes are Required for Efficient Induction of Highly Active CD25⁻ iTreg

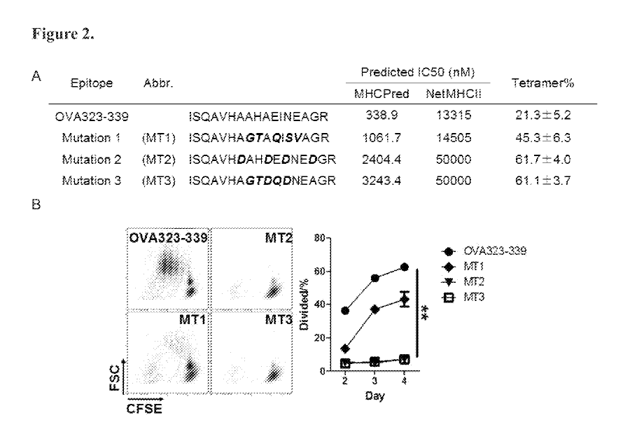

To further determine how antigenicity affects CD25⁻ iTreg induction, a set of mutated epitopes were generated from OVA₃₂₃₋₃₃₉ (SEQ ID NO:187). Using a tetramer staining-based epitope competition assay, the affinity of each of the mutated epitopes for MHC II was assessed. The result showed the order of affinity to be OVA₃₂₃₋₃₃₉>MT1>MT2=MT3 (FIG. 2A) (SEQ ID NO:187). Consistent with this result, in vitro T cell proliferation assays using DO11.10 CD4⁺ T cells showed a similar order in T cell stimulating activity (FIG. 2B). Selected the epitopes OVA₃₂₃₋₃₃₉, MT1, and MT2 as probes for antigenicity studies were therefore selected.

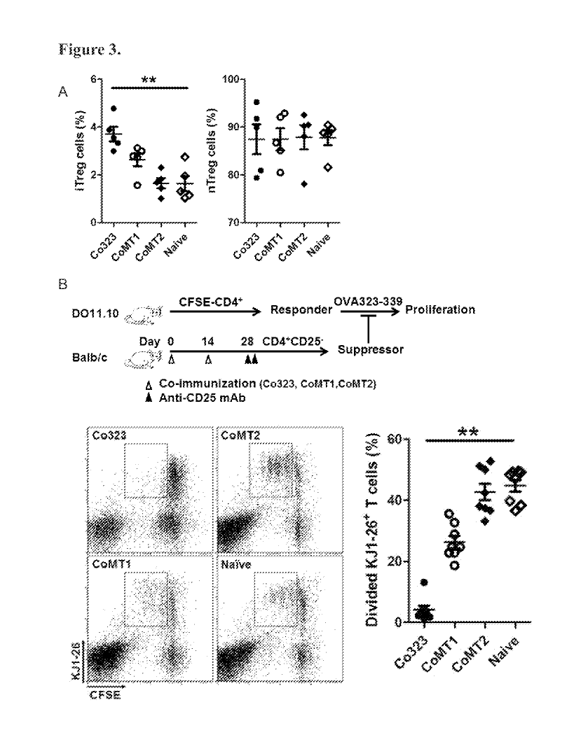

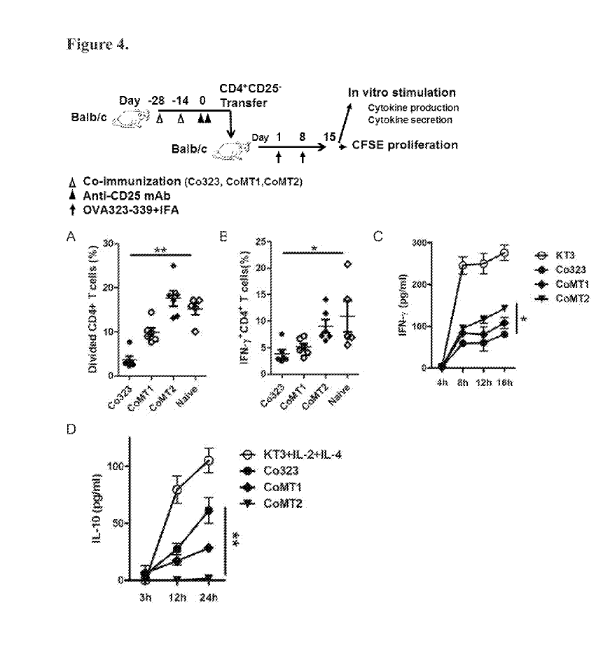

To that end, Balb/c mice (1-Ad⁺) were treated by co-immunization using the DNA and protein combination corresponding to the OVA₃₂₃₋₃₃₉ (SEQ ID NO:187), MT1, or MT2 epitope (designated as Co323, CoMT1, or CoMT2). Seven days after the treatment, splenocytes were isolated and analyzed for CD25⁻ iTreg induction. When compared to untreated control mice (FIG. 3A), the treated mice showed increased frequency of Foxp3⁺ cells in the CD4⁺CD25⁻ (CD25⁻ iTreg), but not the CD4⁺CD25⁺ (nTreg) cell population. Importantly, the magnitude of increase followed the order of Co323>CoMT1>CoMT2, suggesting that efficient induction of CD25⁻ iTreg by co-immunization requires highly antigenic epitopes.

To further determine the impact of antigenicity on the function of CD25⁻ iTreg, the suppressive activity of CD25⁻ iTreg induced by Co323, CoMT1, and CoMT2 were compared using an in vitro suppression assay. All CD25⁻ iTreg cells suppressed the OVA₃₂₃₋₃₃₉ specific proliferation of reporter CD4⁺ T cells in co-culture as expected. However, their relative suppressive activity followed the same order of Co323>CoMT1>CoMT2 (FIG. 3B), suggesting that more antigenic epitopes also induced functionally more active CD25⁻ iTreg cells.

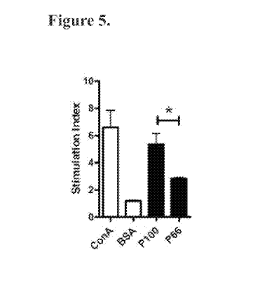

To repeat this observation in vivo, CD25⁻ iTreg induced with the different epitopes were adoptively transferred into Balb/c mice, and then an attempt was made to sensitize the animals with OVA₃₂₃₋₃₃₉ in incomplete Freund's adjuvant (IFA). One week later, splenic CD4⁺ T cells were isolated from the sensitized mice and recall activation of CD4⁺ T effector cells was measured by an in vitro restimulation assay. Although all transferred CD25⁻ iTreg blocked the recall proliferation of T cells to some degree, their relative effectiveness varied with the inducing epitopes, in the order of Co323>CoMT1>CoMT2 (FIG. 4A). These results were similar to those seen in vitro. Moreover, splenic CD4⁺ T cells isolated from the recipients showed decreased expression of IFN-γ and increased expression IL-10, the extent of which also followed the same order (FIG. 4, B-D). Taken together, these results show that highly antigenic epitopes are required for more efficient induction of highly suppressive CD25⁻ iTreg.

### Example 4

### Highly Antigenic Epitopes are Also Required for More Effective Prevention of Flea Allergic Dermatitis

Flea allergic dermatitis is an allergic reaction to flea allergen that is mediated by CD4⁺ T effector cells. To the above findings to a disease model, two antigenic epitopes from the flea allergen FSA1 were chosen, namely P66 (amino acids 66-80) (SEQ ID NO:189) and P100 (amino acids 100-114) (SEQ ID NO:188). P100 is predicted to have a higher affinity to MHC II (1-Ab) than P66. This prediction was confirmed by sensitizing C57BL/6 mice (1-Ab⁺) with full-length FSA1 followed by an in vitro restimulation assay using one of the epitopes. P100 indeed induced significantly more vigorous T cell proliferation than did P66 (FIG. 5).

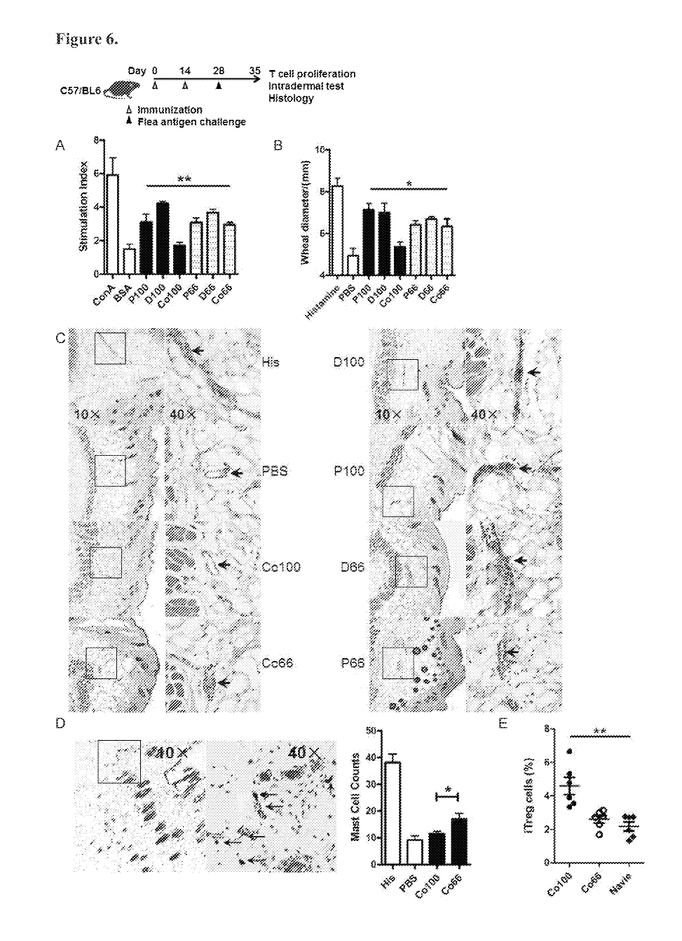

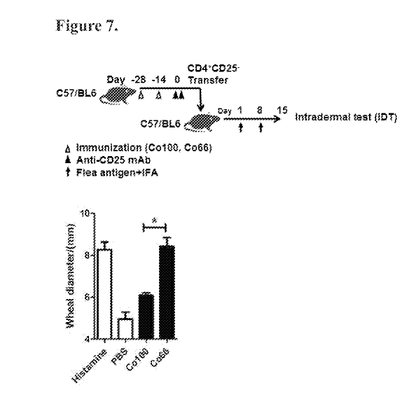

To see whether the difference in antigenicity influences the induction of CD25⁻ iTreg cells by these two epitopes, C57BL/6 mice were prophylactically treated with co-immunization using the combination of DNA and protein vaccines targeting each epitope (designated as Co100 or Co66). Seven days after co-immunization, the animals were sensitized with flea saliva extracts, followed by a delayed-type hypersensitivity assay to determine to which extent the prophylactic co-immunization prevents the development of an allergic reaction. Both the size analysis and histological examination showed a stronger protective effect by Co100 than by Co66, as indicated by smaller wheal diameters (FIG. 6B) and fewer mononuclear infiltrates (FIG. 66) at the reaction site. The Co100-treated mice also had fewer mast cells and a lower level of degranulation at the reaction site (FIG. 6D). In vitro recall activation also confirms weaker T cell response in the Co100 group (6A). Importantly, P100 also induced more CD25⁻ iTreg than P66 (FIG. 6E), suggesting that P100 protects animals more effectively by inducing more CD25⁻ iTreg.

To determine whether this is indeed the case, CD25⁻ iTreg cells induced by Co100 or Co66 were adoptively transferred into FSA1-sensitized mice and challenged the recipients with flea antigens. Again, recipients receiving Co100-induced CD25⁻ iTreg cells showed significantly reduced DTH response than those receiving Co66-induced counterpart (FIG. 7). Collectively, these results confirm in this disease model that highly antigenic epitopes are required for more efficient induction of therapeutic CD25⁻ iTreg.

The above results establish that efficient induction of highly active CD25⁻ iTreg cells requires highly antigenic epitopes for T cells. The finding is based on 1) anti-MHC-II mAb blocked the induction CD25⁻ iTreg cells in vitro (FIG. 1); 2) OVA³²³⁻³³⁹ mutants with decreased antigenicity for T cells showed decreased ability to induce active CD25⁻ iTreg cells (FIGS. 2-4); and 3) a similar observation was made in a mouse model of flea allergic dermatitis, where CD25⁻ iTreg cells induced by a more antigenic epitope were also more effective in preventing the development of the disease (FIGS. 5-7).

iTreg cells are potentially useful as therapeutics for allergy, autoimmune diseases, and transplant rejection. The present study thus has the translational importance by uncovering the need for choosing highly antigenic epitopes for effective induction of CD25⁻ iTreg. At present, immunosuppressant treatment is the only means to control immune disorders and pathology, which is unfortunately associated with many side effects, including increased risk of infection and cancer. In vivo induction of CD25⁻ iTreg cells, which are antigen-specific, provides a means of controlling immune diseases while avoiding global immunosuppression. Highly therapeutically effective CD25⁻ iTreg can be induced by co-immunization targeting one or several disease-related or specific antigens, and by selecting antigenic epitopes of highest antigenicity for T cells as the immunogen.

### Example 5

### Methods

### Animal and Reagents

Balb/c and C57/B6 mice were purchased from Beijing Vital Laboratory Animal Technology Company, Ltd. (Beijing, China) and Balb/c, DO11.10 were from SLAC Laboratory Animal (Shanghai, China) and maintained under pathogen-free conditions. Peptides were synthesized by Scipeptide Ltd. (Shanghai, China). Antibodies for flow cytometry were purchased from BD Biosciences (CA, USA). Flea saliva extracts were purchased from China Medicines Corporation (Beijing, China).

Epitope Design

The dominant epitope of hen ovalbumin for I-Ad (OVA₃₂₃₋₃₃₉: ISQAVHAAHAEINEAGR) (SEQ ID NO:187) was mutated as reported and predicted with online servers MHCPred and NetMHCII, both of which are well-known in the art. The epitopes of flea salivary antigen 1 (FSA1, Swiss-Prot: □94424.3) for I-Ab (P100: GPDWKVSKECKDPNN (SEQ ID NO:188)) and P66: QEKEKCMKFCKKVCK (SEQ ID NO:189)) were selected using MHCPred. Corresponding DNA vaccines coding for OVA₃₂₃-339, MT1, MT2, P100, and P66 were constructed with the pVAX1 vector, designated as pVAX1-OVA, pVAX1-MT1, pVAX1-MT2, D100, and D66.

Antigen Sensitization

Mice were immunized by subcutaneous injection (s.c.) twice on days 0 and 7 with 100 ug peptide emulsified in 100 ul IFA (Sigma-Aldridge Inc. San Louis, USA).

Tolerogenic Co-Immunization

Balb/c mice were injected intramuscular (i.m.) on days 0 and 14 with 100 ug each of OVA₃₂₃₋₃₃₉ and pVAX1-OVA, MT1 and pVAX1-MT1, or MT2 and pVAX1-MT2. C57BL/6 mice were similarly injected with P100 and D100, or P66 and D66.

MHC-II Blocking

Purified CD4⁺ T cells (5×10⁵, R&D System, Minneapolis, USA, MAGM202) from Balb/c D011.10 mice or OVA₃₂₃₋₃₃₉ sensitized Balb/c mice were cultured with purified DCs (1×10⁵, Miltenyi Biotec, Gladbach, Germany, 130-052-001) from co-immunized (pVAX1-OVA plus OVA₃₂₃₋₃₃₉) Balb/c mice. The cells were cultured for 7 days with or without anti-MHC II mAb (M5/114.15.2, eBioscience, San Diego, USA).

Flow Cytometry

CD4⁺CD25⁻Foxp3⁺ iTreg were detected by immunostaining with anti-CD4-FITC, anti-CD25-APC, and anti-Foxp3-PE mAbs. Intracellular IFN-γ was detected in monensin-blocked and anti-CD3 and anti-CD28 stimulated T cells by intracellular staining with anti-IFN-γ-PE mAb. Data were collected with a BD FACSCalibur and analyzed with Flowjo (Tree Star, Ashland, USA). The supernatant of cultured T cells was also analyzed for IFN-γ and IL-10 using the FlexSet Beads Assay (BD Biosciences).

Tetramer Competition Assay

PE-conjugated OVA₃₂₃₋₃₃₉-loaded I-Ad tetramer (NIH Tetramer Core Facility) was competed with OVA₃₂₃₋₃₃₉ or a mutant peptide by incubation of 2×10⁵ DO 11.10 T cells, the OVA₃₂₃₋₃₃₉ tetramer, and a competing peptide together for 5 minutes. Five volumes of medium with 10% FCS were added to stop the competition. Cells were washed 3 times and immediately analyzed for PE-positive T cells by flow cytometer.

T Cell Proliferation

MTT-based and CFSE-based T cell proliferation assays were performed as described before.

In Vitro Suppression Assay

OVA₃₂₃₋₃₃₉-specific CD4⁺ T cells from DO11.10 mice spleen were labeled with CFSE (responder cells) and co-cultured with co-immunization-induced CD4⁺CD25⁻ T cells at a 1:1 ratio (2×10⁵ each). OVA₃₂₃₋₃₃₉-specific proliferation of the responder cells was analyzed by CFSE dilution on day 4 using a FACScalibur. To block nTreg in vivo, two 10 ug dose of anti-CD25 mAb (clone 3c7, eBioscience) were injected intravenously (i.v.) into co-immunized mice at −48 h and −24 h before CD25⁻ iTreg isolation.

In Vivo Suppression Assay

Balb/c mice were injected (i.v.) with co-immunization-induced CD25⁻ iTreg (2×10⁶) on day 0. On days 1 and 8, the mice were repeatedly sensitized for the same antigen. On day 15, the mice were sacrificed and splenic T cells were isolated and analyzed for recall activation by the T cell proliferation assays.

Intradermal Test (IDT) and Histology

Antigen-sensitized C57BL/6 mice were challenged intradermally (i.d.) with 10 ug of FSA (Greer Laboratories) on the nonlesional lateral thorax skin. PBS is used as a sham control and histamine is used as a positive control. The diameter of the skin reaction was measured within 30 min after challenge using a calibrated micrometer Skin samples were collected within 30 min of antigen challenge, fixed in 4% paraformaldehyde, embedded in paraffin, and sectioned. Antigen retrieval was accomplished by boiling the slides in 0.01M citrate buffer (pH 6.0), followed by staining with H&E for T cells or toluidine blue for mast cells.

Statistical Analysis

Pair-wise comparison was made using the Student's t test. Comparison among three or more groups was made by the ANOVA test. Difference is considered statistically significant if p<0.05.

### Example 6

### Distinct Roles of TGF-β and IL-10 in Development and Suppressive Function of CD4⁺CD25⁻Foxp3⁺ iTreg Induced by DNA and Protein Vaccines Against Asthma

Co-immunization of DNA vaccine and cognate protein together can induce tolerogenic dendritic cells that could further induce Foxp3 expression in CD4⁺CD25⁻ T cells and prevent several allergic or autoimmune diseases in murine models. This example demonstrates the immunoregulatory effect of the co-immunization-induced and iTreg mediated suppression in a dust mite-induced allergic asthma by co-inoculating DNA encoding the Derp1 antigen and Derp1 protein. The results show that co-immunization not only contribute to significant limit the inflammatory responses in the lungs, but also to the inhibition of Th2 cytokines and production of IgE. Furthermore, the suppression is mediated by the induction of CD4⁺CD25⁻Foxp3⁺ iTregs via suppressive cytokines such as IL-10, but not the cell-cell contact. Additionally, the conversion of iTregs from naïve T cell can be initiated by TGF-β1 secreted from the tolerogenic DCs 3 days after co-immunization. This induction of Foxp3 expression in the naïve T cells could be demolished after the blockade of TGF-β1. Simultaneously, autocrine IL-10 can strengthen the suppressive ability of TGF-β mediated iTregs via IL-10R on DCs. In vitro, the TGF-β1 could also induce the Foxp3 expression in the CD4⁺CD25⁻ naïve T cells in the present of anti-CD3/anti-CD28. Thus, this co-immunization protocol induces TGF-β1 and IL-10 secreting tolerogenic DCs that further convert naive T cell into the iTregs.

Airway hyperresponsiveness is a major pathophysiological characteristic of bronchial asthma can be caused by environmental aeroallergens. One of the major aeroallergens is the house dust mite (HDM) that has been proved to contribute to both immediate hypersensitivity and chronic asthma in lung. The most important allergen is Dermatophagoides pteronyssinus (Der-p1), a cysteine protease derived from the mite's intestinal tract. Patients allergic to Der-p1 have been demonstrated to have elevated serum levels of allergen-specific IgE and provoked local infiltration of inflammatory cells. In recent years, general knowledge regarding the regulation of asthma and allergen immunotherapy by T regulatory cells (Tregs) has rapidly developed.

T regulatory cell (Treg) is one of key suppressive and homeostatic components in immune system and maintains immunologic tolerance to auto-antigens in various immune disorders such as autoimmune diseases, chronic viral infections, and cancer. T regulatory cells, including the naturally occurring thymus derived CD4⁺CD25⁺ Treg cells, adaptive Tr1 and mucosal induced Th3 cells have been proposed to be used in clinical trial. A novel sub-population of Treg characterized with CD4⁺CD25⁻Foxp3⁺ has been recently discovered in aged mice or systemic lupus erythematosus (SLE) patients. In previous studies, it has been demonstrated that co-immunization with protein antigen and plasmid DNA coding the same antigen into mice could induce Foxp3 expression in CD4⁺CD25⁻ T cells. The mechanism of how this subtype of iTregs functions, however, is unknown. Tregs control immune responses through several mechanisms, including production of suppressive cytokines such as IL-10 and TGF-β; cell-cell contact dependent inhibition mediated by the negative regulators of CTLA-4, GITR and PD-1; induction of semimature DC. In this example it is shown that the suppressive ability of these iTregs required IL-10, but not the TGF-β or cell-cell contact to inhibit effector T cells response.

TGF-β1 and IL-10 not only are a critical suppressive cytokine involved in the induction of immune tolerance, but also can convert peripheral naive T cells to Tregs in the present of anti-CD3/anti-CD28. In this example, it is established that co-immunization induced immature dendritic cells (DC) into DCreg, which also could secrete IL-10 and TGF-β and convert naive T cell into the iTregs in vivo. The induction of these iTregs was demolished by neutralization of TGF-β secreted by DC and the suppressive ability was decreased when defience of IL-10 signal.

Therefore, it is demonstrated that in the dust mite-mediated asthma model in rodents, the clinical onsets and allergic responses are significantly improved by the co-immunization of Der-p1 DNA vaccine and Der-p1 protein. The mediation of suppression is also demonstrated by the antigen specific CD4⁺CD25⁻Foxp3⁺ iTregs. Furthermore, TGF-β1 and IL-10 play distinct roles in the induction and suppressive ability of CD4⁺CD25⁻Foxp3⁺ iTregs.

### Example 7

### Materials and Methods

Vaccine preparations. The DNA sequence from full length of Dermatophagoides pteronyssinus 1 (Der-p1, GeneBank Access No. EU092644) was synthesized and cloned into pVAX1 vector (Invitrogen Inc. USA). Recombinant Der-p1 protein was cloned into pET28a and expressed in E. coli system. The pVAX-Der-p1 expression was identified by RT-PCR analysis from the total RNA of transfected BHK21 cells after 72 h. The Der-p1 protein was purified from pET28a-FSA1 transformed E. coli BL21(DE3) according to a previous protocol. Plasmids and recombinant proteins were dissolved in saline at 1 mg/ml and stored at −80° C. before use.

Mice and immunization. Female C57BL/6 and BALB/C mice at 6-8 weeks old were purchased from Animal Institute of Chinese Medical Academy (Beijing, China). Balb/c.Foxp3^{gfp} mice were purchased from the Jackson Laboratory. All mice were received pathogen-free water and food. C57BL/6, BALB/C.Foxp3^{gfp} mice were immunized with plasmid DNA at 100 μg/animal, or protein at 100 μg/animal, or a combination of both at 100 μg each/animal as the vaccine regimens, respectively, into tibialis anterior muscle on days 0 and 14.

HDM-induced Allergic Pathogenesis. Allergen-induced asthma was induced as described previously. C57BL/6 mice were immunized by i.p. injection with 4000 U of HDM antigens (Greer Laboratories, Lenoir, N.C.) in 0.1 ml PBS or PBS alone at days 1 and 7, followed by intratracheal challenge with 2000 U of HDM antigens in 100 μl PBS or an equivalent volume of PBS as a control at days 14, 16, 18, 20 and 22. One day after the last challenge, BALs were collected, and tissues were harvested for immunohistopathologic analysis or cultures in vitro.

Histology analysis. Twenty-four hours after the last intratracheal challenge, lung samples from mice were collected from each group and fixed in 4% paraformaldehyde and embedded in paraffin blocks. Sections were then cut and fixed. Antigen retrieval was accomplished by boiling the slides in 0.01M citrate buffer (pH 6.0) followed by staining with hematoxylin and eosin (H&E) and analyzed under a light microscope for determining histology changes.

Measurement of Der-p1-specific IgE. Serum samples were collected and examined for the level of Der-p1-specific antibodies by ELISA. The 96-well plates were coated with recombined Der-p1 protein 4° C. overnight. After washing with PBST, the sera were added and incubated for 1 hour at 37° C., then detected with specific horseradish peroxidase-conjugated rabbit anti-mouse IgE antibodies (SouthernBiotech, Birmingham, USA). The absorbance at 450 nm was measured using an ELISA plate reader (Magellan, Tecan Austria GmbH).

Flow cytometric (FACS) analysis. For intracellular staining, T cells were stimulated with Der-p1 protein (10 μg/ml) for 8 hrs and subsequently treated with monensin (3 μM) for 2 hrs in vitro. The cells were blocked with Fc-Block (BD Phamingen, San Diego, USA) in PBS for 30 min at 4° C. before fixed with 4% paraformaldehyde and permeabilized with saponin. The cells were intracellularly stained with the appropriate concentrations of antibodies including APC-labeled anti-Foxp3, PECy5-labeled anti-CD25, FITC-labeled anti-CD4, PE-labeled anti-IL-10, PE-labeled anti-GITR, PE-labeled anti-CTLA4, PE-labeled anti-PD-1 antibody 30 min at 4° C., respectively. The cells were analyzed with a FACScalibur using the Cell QuestPro Software (BD Bioscience).

In vitro proliferation/inhibition assays. In proliferation assays, single lymphocyte suspensions were obtained from spleens of each group on 7 days after the second immunization. T cell proliferation was performed by MTT method after the Der-p1 (10 μg/ml) or PMA (50 ng/ml)/ionomycin (500 ng/ml) stimulation in vitro for 48 hrs. For suppression assays, CD4⁺CD25⁻GFP⁺, CD4⁺CD25⁺GFP⁺ and CD4⁺CD25⁻GFP⁻ T cells were purified by a high-speed cell sorter (MoFlo Cell Sorter, Beckman Coulter, USA) with PE-labeled anti-CD4 and APC-labeled anti-CD25. The sorted cell purity was examined and over 97% was achieved. Purified suppressor T cells (4×10⁴ or 2×10⁴) were co-cultured with CD4⁺CD25⁻ responder T cells (2×10⁵) obtained from BALB/C mice previously primed with the recombinant Der-p1 emosulfied in CFA (Complete Freund's Adjuvant), and boosted once with the recombinant Der-p1 emosulfied in IFA (Incomplete Freund's Adjuvant). Responder T cells were stimulated with Der-p1 (10 μg/ml) and APC (1×10⁴) in 96-well plates for 72 hrs. Following stimulation, cell proliferation was assessed by a colorimetric reaction after the addition of 20 μl of an MTT-PMS (Pormaga, USA) solution for 4 hrs. Its color density was determined at 595 nm by a 96-well plate reader (Magellan, Tecan Austria GmbH) 5 min after adding 100 μl DMSO (AMRESCO, USA).

Transwell experiments. Transwell experiments were performed in 24-well plates. CD4⁺CD25⁻ responder T cells (1×10⁶) isolated as above were stimulated with Der-p1 (10 μg/ml) and APC (2×10⁵) in the lower transwell in the absence or present of anti-IL-10 and anti-TGF-β. Purified CD4⁺CD25⁻GFP⁺ iTregs (2×10⁵), CD4⁺CD25⁺GFP⁺ nTregs (2×10⁵) and CD4⁺CD25⁻GFP⁻ T cells were cocultured with APC (4×10⁴) in the upper transwell chambers (0.4 μm; Millipore, USA). After 3 days cell proliferation was assessed by MTT method as above.

Analysis of cytokine production. Suppressive cytokines expressed by CD11C⁺ dendritic cells were detected by RT-PCR. Total RNA was isolated from CD11C⁺ cells of C57BL/6 mouse spleens 3 days after the first co-immunization using TRIzol reagent (Promega). cDNA was synthesized and PCR was performed with each of the following primers: GAPDH, TGF-β1, IL10, RALDH1, RALDH2, RALDH3. RT-PCR was performed with each primer according to the manufacturer's instructions (TaKaRa RNA PCR Kit). Cytokines in serum from treated or untreated mice induced asthma model were measured by IL-4, IL-5, IL-10 and IL-13 cytometric bead assay Flex Sets (BD Bioscience) according to the manufacturer's instructions.

Blockade of TGF-β1 or IL-10 in vivo. To measure the effect of TGF-β1 on induction of iTregs in vivo, C57BL/6 mice were injected i.p. with 400 μg per injection of anti-TGF-β1 mAb (2G7), anti-IL-10 mAb (JES-2A5) or with an isotype-matched mouse immunoglobulin G1 (IgG1) as a control in 0.5 ml phosphate-buffered saline (PBS) for three consecutive days after each co-immunization. Neutralizing function of anti-TGF-(31 mAb and anti-IL-10 mAb was measured in serum using the Emax immunoassay system (Promega, Madison, Wis.) or IL-10 cytometric bead assay Flex Sets (BD Bioscience) according to the manufacturer's protocol.

In vitro T cell priming assays. To generate CD4⁺CD25⁻Foxp3⁺ cells in vitro, Naive CD11c⁺ dendritic cells (2×10⁵) were cultured in 6-well, and stimulated with pVAX-Derp1 (10 μg/ml) plus Derp1 peotein (10 μg/ml) in the present of anti-IL10 or TGF-β for 48 hrs. Three groups of dendritic cells pretreated were added to culture medium with naive CD4⁺CD25⁻ T cells (1×10⁶) in RPMI 1640 each 48 hrs for 3 times. And then GFP expression in CD4⁺CD25⁻ T cells were analyzed by FACS. To check the roles of cytokines during DCreg induce iTregs, we co-cultured CD11c⁺ DC pretreated with pVAX-Derp1 (10 μg/ml) plus Derp1 peotein (10 μg/ml) with naive T cells, synchronously, plus anti-IL-10, anti-TGF-β or TGF-β receptor inhibitor, SB-525334 (14.3 nM) each 48 hrs for 3 times. In order to detect the ability of TGF-β and IL-10 to induce the CD4+CD25−Foxp3+ Tregs, naive CD4+CD25− T cells (1×10⁶) were stimulated with plate-bound anti-CD3 (3 μg/ml)/anti-CD28 (1 μg/ml) in the presence or absence of titrated rhTGF-β1 or rmIL10 (PeproTech, USA).

Western blot for NFAT1 and NFAT2. Purified CD4⁺CD25⁻GFP⁺, CD4⁺CD25⁺GFP⁺ Tregs or CD4⁺CD25⁻GFP⁻ T cells (5×10⁶) in RPMI were fractionated with NE-PER nuclear or cytoplasmic reagent kit (Pierce Biotechnology, Inc., Rockford, Ill., USA). Lysates were subjected on 8.0% SDS-PAGE gels, transferred to nitrocellulose membranes, and then blocked with a 5.0% milk solution in TBS with 0.1% Tween. Membranes were then probed with anti-mouse NFAT1, NFAT2, GADPH and Histone (all from Santa Cruz Biotechnology, Santa Cruz, Calif., USA).

Statistical analysis. Statistical analyses are performed using the Student's t-test. In these analyses, the data is converted into log. If the P<0.05, the data indicated significant differences.

### Example 8

### Co-Immunization Suppresses the Development of HDM-Induced Allergic Asthma

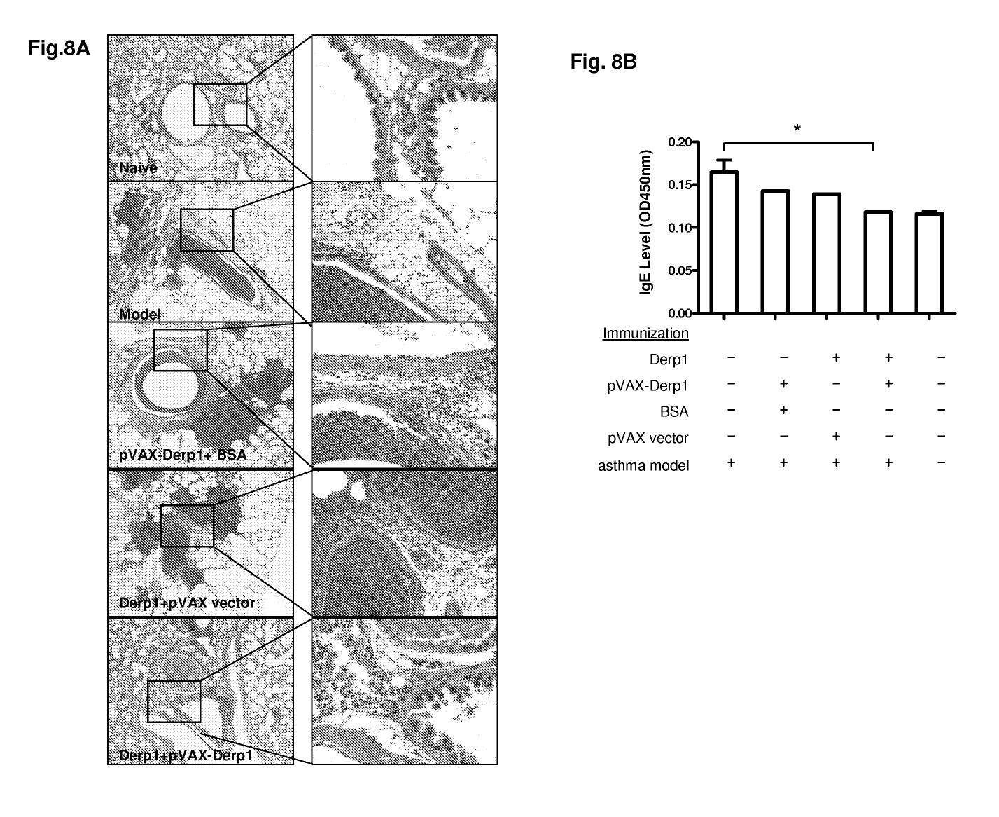

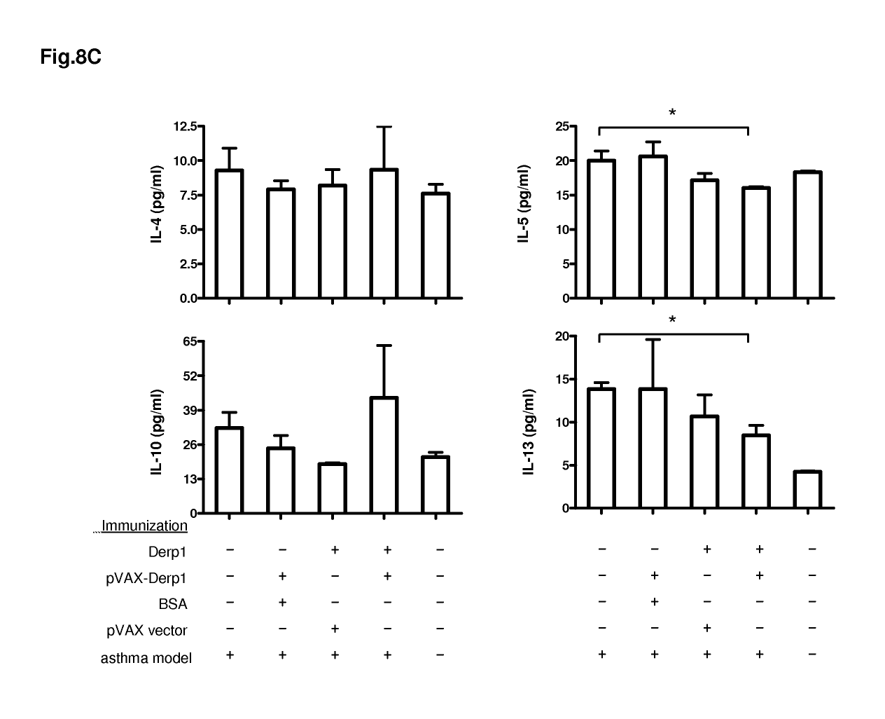

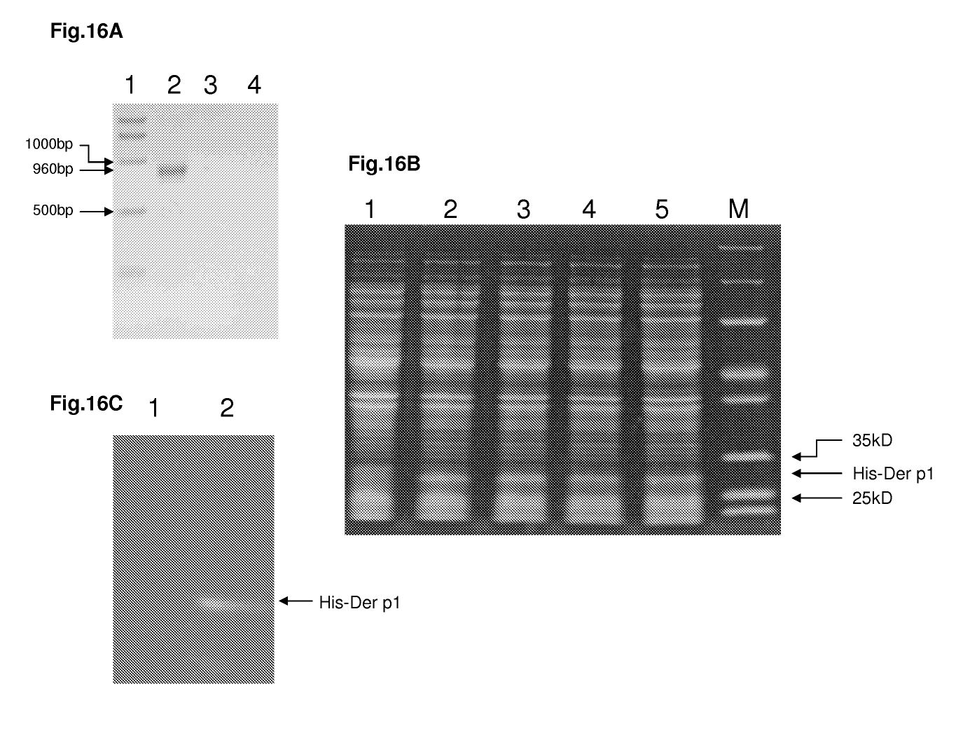

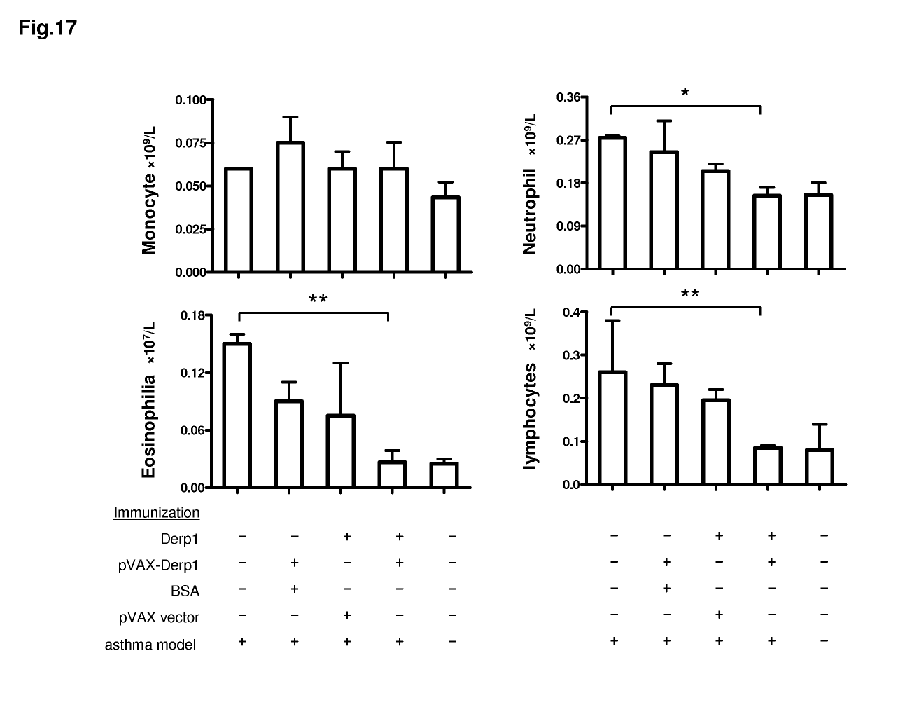

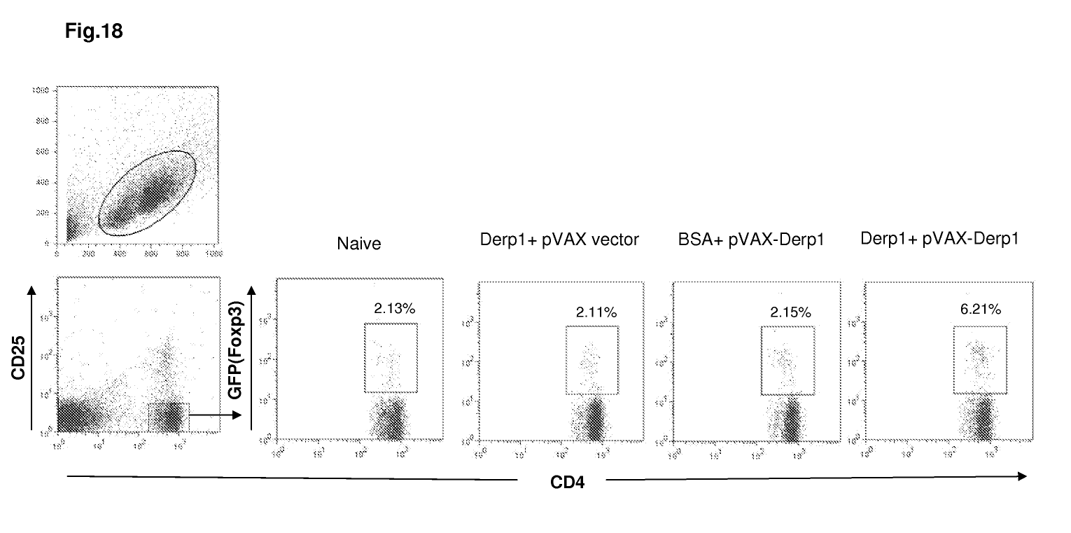

To demonstrate the efficacy of co-immunization with DNA and recombinant protein vaccines in protecting against asthma, DNA and protein vaccines that were based on the sequence of dust mite allergen, Dermatophagoides pteronyssinus 1 (Derp1, FIGS. 16A-C) were cloned and constructed, and then tested in the dust mite-mediated asthma or AHR in mice. C57BL/6 mice were pre-treated with the pVAX-Derp1 DNA vaccine and recombinant Der-p1 protein as the co-immunized group (pVAX-Derp1+Derpl) or other immunogens intramuscularly twice at biweekly intervals. In order to eliminate the influence of unrelevant vector and protein on the response, mice were co-immunized with pVAX-Derp1+BSA, or Derp1 protein+pVAX vector, as the mismatched co-immunization controls. Subsequently, all animals except the negative control were induced and intratrecheal challenged with HDM to induce the asthma as previously described. Histological analysis revealed massive inflammatory cell infiltrations in the lung (FIG. 8A) in the un-treated mice as the indication of successful induction of the AHR compared with the lung tissues in PBS-injected negative control mice. The mice pretreated with the co-immunization exhibited a significant reduction of the inflammatory cell infiltrations and normal lung structures (FIG. 8A). The percentage of different cell subtypes in bronchoalveolar lavage (BAL) was analyzed 24 hrs after the last challenging. Eosinophils, neutrophils and lymphocytes were reduced in the co-immunized mice significantly and consistently with observations above (FIG. 17).

Since allergic antigens trigger IgE that can mediate AHR, it was investigated if the pVAX-Derpl+Derpl could inhibit induction of anti-Der-p1 IgE. The level of ant-Der-p1-specific IgE was therefore measured 24 hrs after the last intratracheal challenge. Its level was significant reduced in the co-immunized mice compared with the model group (FIG. 8B).

High level of Th2 related cytokine productions, including the IL-4, IL-5 and IL-13 have been demonstrated to associate with the severity of allergic responses, the level of these cytokines in sera were measured by Flex set. Mice from the model group, mismatched group are induced to produce higher level of IL-5 and IL-13 (FIG. 8C); whereas, mice pretreated with pVAX-Derp1+Derp1 produced relatively low level of these cytokines, but high level of IL-10, suggesting that the co-immunization induces a preventive effect to allergic responses. Thus, co-immunization induced suppression could dampen inflammation and its disease-associated cytokine productions in vivo.

### Example 9

### CD4⁺CD25⁻Foxp3⁺ iTregs Contribute to the Immune Toleration Induced by Co-Immunization

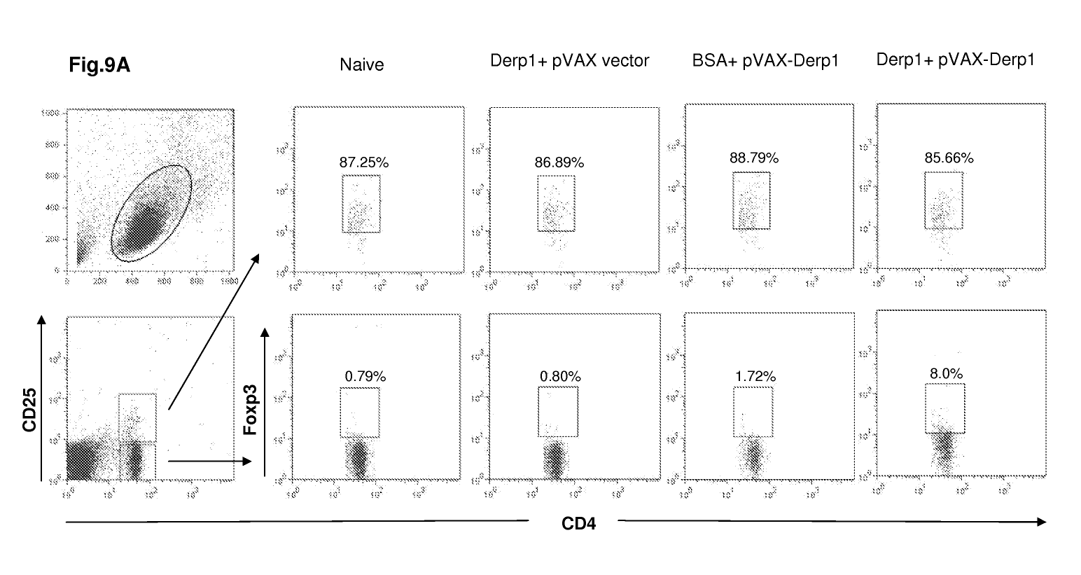

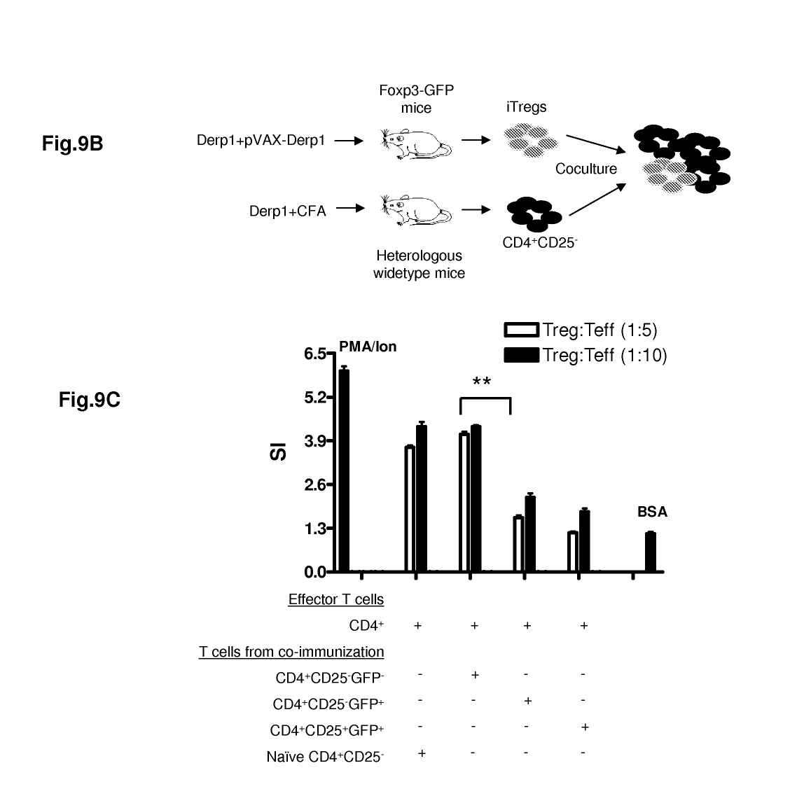

To examine if pVAX-Derp1+Derp1 co-immunization could up-regulate Foxp3 expression, the percent of CD4⁺CD25⁻Foxp3⁺ or CD4⁺CD25⁺Foxp3⁺ T cells was analyzed by FACS 7 days after the second co-immunization. As shown in FIG. 9A, the population of CD4⁺CD25⁻Foxp3⁺ T cells was increased in the mice co-immunized with the pVAX-Derp1+Derp1 compared with other groups, suggesting the inducible Treg cells elicited. In agreement with previous findings, no changes in Foxp3 expression were observed, although at high levels, changes were observed in CD4⁺CD25⁺ nTreg cells among the groups, arguing against the notion that nTreg cells might be also contributed to the suppression.

In order to examine whether CD4⁺CD25⁻Foxp3⁺ iTregs contribute to suppression in co-immunization, the CD4⁺CD25⁻ cells were purified and then sorted the Foxp3⁺ iTreg cells in MoFlo sorter by using the Foxp3^{gfp} mice after immunized with various regimens including the co-immunizations. The sorted T cells were mixed with responder CD4⁺ T cells isolated from BALB/c mice previously primed with recombinant Derp1 plus CFA and boosted with recombinant Derp1 plus IFA (FIG. 9B). As depicted in FIG. 9C, CD4⁺CD25⁻GFP⁻ T cells did not display any in vitro suppressive function; whereas, both of CD4⁺CD25⁻GFP⁺ and CD4⁺CD25⁺GFP⁺ T cells impaired the proliferative response for the responder T cells at a 1:5 or 1:10 Treg:Teff cell ratio. The result indicates that the immunosuppression is only derived from CD4⁺CD25⁻Foxp3⁺ Treg cells, but not from the other CD4⁺CD25⁻Foxp3⁻ T cells. It further suggests that CD4⁺CD25⁻Foxp3⁺ iTregs induced by co-immunization contribute to the immune toleration.

### Example 10

### IL-10 Maintains Suppressive Function of iTregs Induced by Co-Immunization

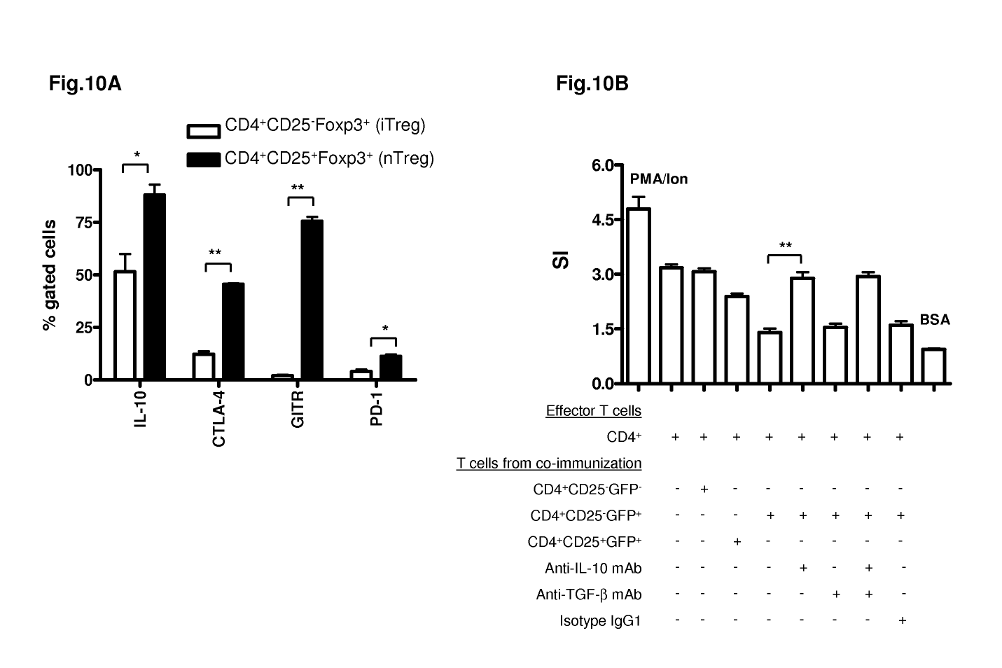

It is notable that the acquisition of suppressive activity in CD4⁺CD25⁻ T cells by co-immunization associated with Foxp3 up-regulation. But it remained unknown whether the suppressive function of iTregs occurred by cell-cell contact or was cytokine-dependent. Firstly, the CD4⁺CD25⁻Foxp3⁺ iTreg cells with a set of specific negative receptors previously used for identification of Treg populations were characterized. It was observed that the IL-10 expressing CD4⁺CD25⁻Foxp3⁺ iTreg cells displayed a low expression of CTLA4, GITR and PD-1 on the surface (FIG. 10A), which is distinguishable from previous identified nTreg and Tr1 cells. This indicated that the suppressive function of iTregs is not dependent on a cell-cell contact mechanism. In order to confirm this hypothesis, CD4⁺CD25⁻GFP⁺ iTregs were separated from responder T cells in the transwell plate, and the proliferation level of antigen specific responder T cells was then detected. As shown in FIG. 10B, T effectors were also not able to proliferate, indicating that the non-contact inhibition contribute to iTregs-mediated immune toleration. In addition, blockade of IL-10 in this system could significantly reverse their suppressive ability, and TGF-β had little effect on the suppressive function. Lack of cell-cell contact reversed the nTreg-mediated inhibition, implying that nTregs suppressive function is dependent on both cytokine signaling and cell-cell contact. In conclusion, iTregs inhibit the responder T cells mainly via DC-secreting IL-10, but not TGF-β and negative receptors.

### Example 11

### The Distinct Roles of TGF-β and IL-10 in Development of CD4⁺CD25⁻Foxp3⁺ iTregs

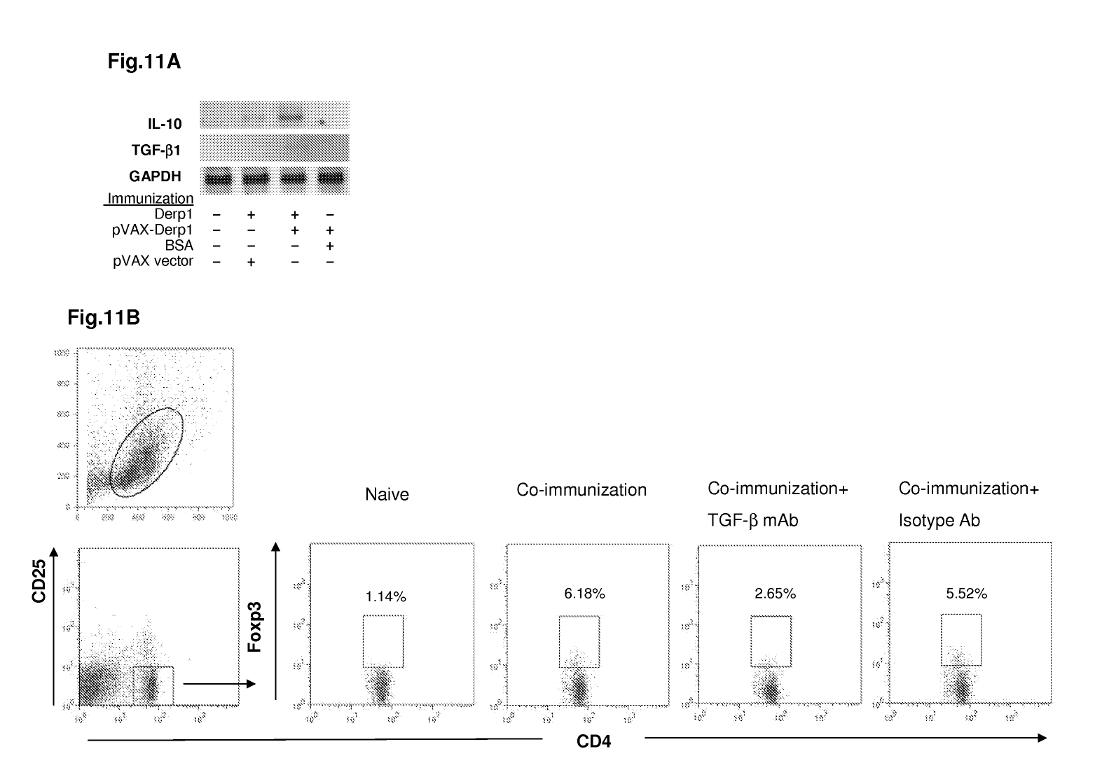

As reported, IL-10, but not the TGF-β is the key mediator of iTregs suppressive function. But whether TGF-β or IL-10 participate in generation of iTregs is still unknown. Some recent reports have suggested that TGF-β1 can promote the development of Tregs by regulating Foxp3 expression, and autocrine IL-10 by dendritic cells can induce long-lasting antigen-specific tolerance in autoimmune or allergic diseases. iTregs have been shown to be detectable 3 days after the first co-immunization, so TGF-β1 and IL-10 expression are measured in CD11c⁺ dendritic cells by RT-PCR assay using the GAPDH (glyceraldehyde-3-phosphate dehydrogenase) as an internal control for RNA levels. As shown in FIG. 11A, the high level of expression for TGF-131 and IL-10 increase in the pVAX-Derp1+Derp1 co-immunized group. As previously reported, retinoic acid can directly promote TGF-β1-mediated Foxp3+ Tregs conversion of naive T cells. The expression level of RALDH1, RALDH2, RALDH3 by RT-PCR was thereby detected, and results show that none of these three retinaldehyde dehydrogenases could be detected in each group (data not shown), suggesting the induction of iTregs may not be elicited by these RA converting enzymes.

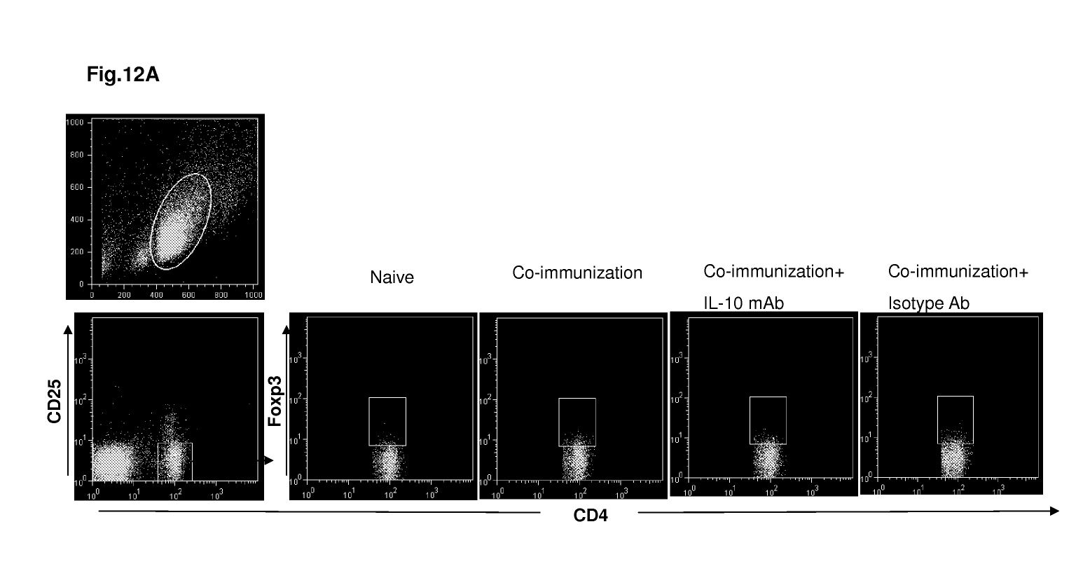

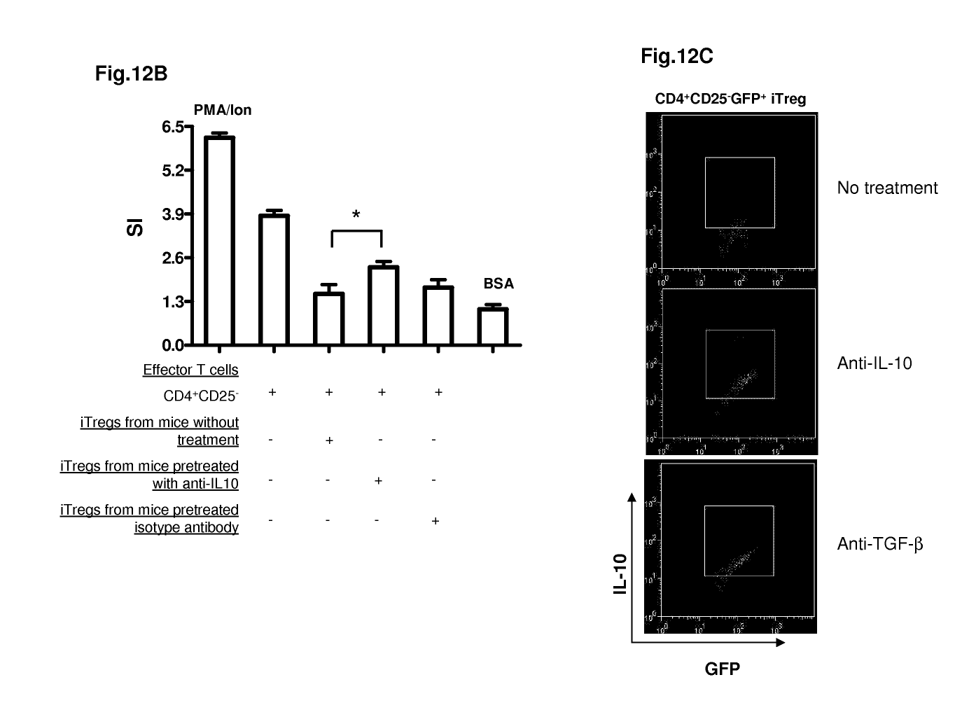

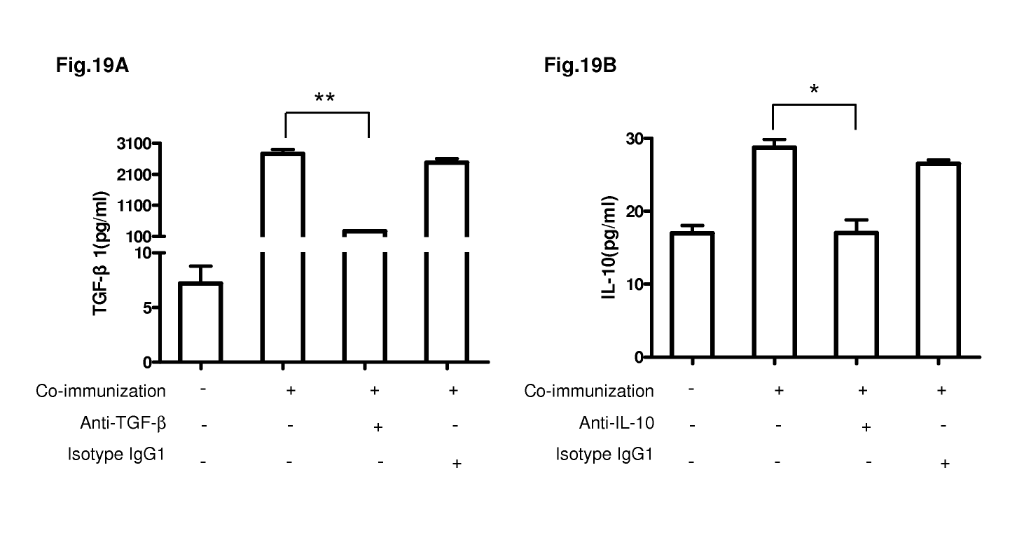

It is of interest to determine if neutralization of endogenously produced TGF-β1 or IL-10 would decrease the induction of iTregs in the co-immunized mice. Mice were given repeated injections of anti-TGF-β1 mAb (2G7), anti-IL-10 (2A5) or isotype control antibodies (IgG1) on days 0-3 after each of two co-immunizations performed in ways known in the art. The neutralizing effects among the groups by the anti-TGF-β1 mAb were analyzed by measuring the TGF-β1 level in serum by ELISA (FIG. 19A) and IL-10 level by Flex Set (FIG. 19B). The mice injected with control antibodies did not affect iTregs development. In contrast, the development of iTreg and immuno-suppression were both reversed in mice injected with anti-TGF-β1 mAb (FIG. 11B), suggesting TGF-β1 is necessary for inducing Foxp3 expression in CD4⁺CD25⁻ iTregs during the co-immunization. To assess the relationship with IL-10, IL-10 was blocked during the initial stage of iTregs. The results show that deficiency of IL-10 signal could not demolish the Foxp3 expression in CD4⁺CD25⁻ T cells (FIG. 12A). Whether these iTregs remained their suppressive function was then examined. To do so, CD4⁺CD25⁻GFP⁺ iTregs were purified from mice pretreated with anti-IL10 mAb and co-cultured with responder CD4⁺ T cells. The results show that blockade of IL-10 signal could partially demolish the iTregs function (FIG. 12B) and this down-regulation was related to the reduction of IL-10 secreted by iTregs (FIG. 12C).

### Example 12

### TGF-β1 Secreted by DC Converts Naïve T Cells into iTregs Directly

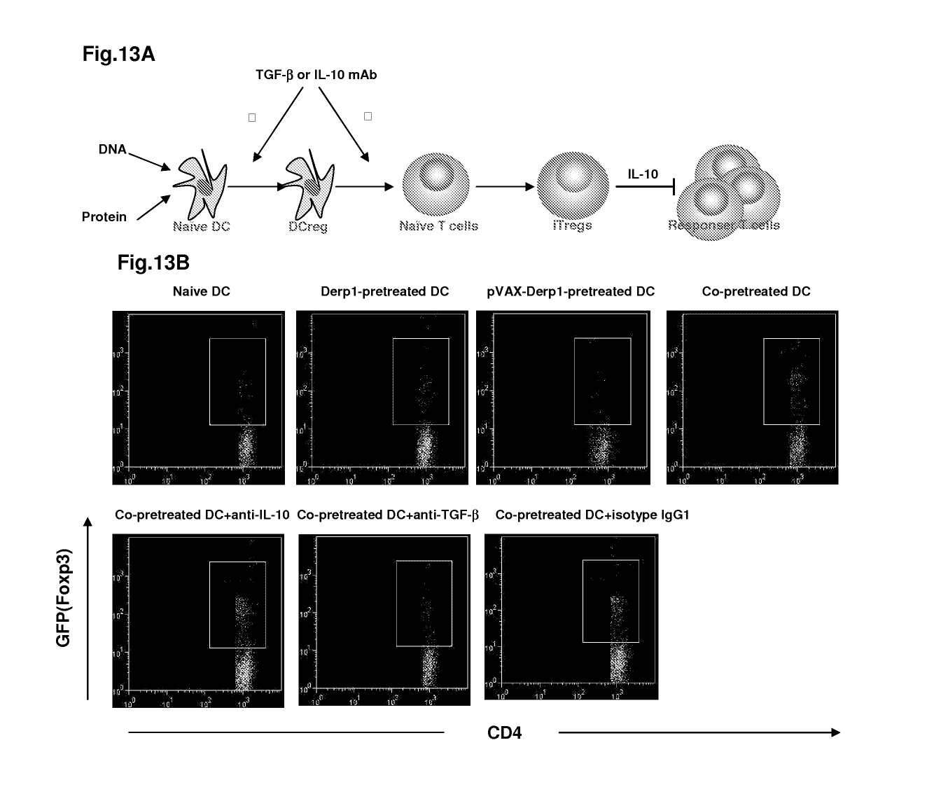

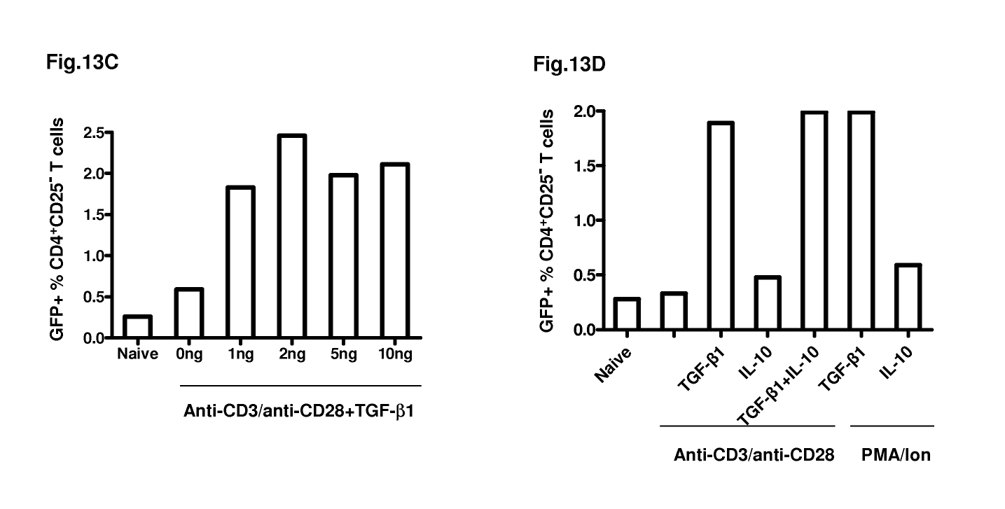

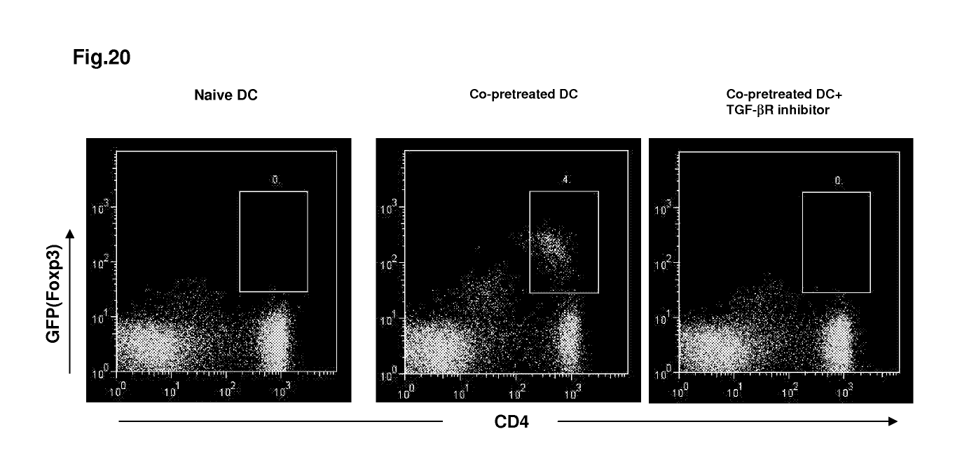

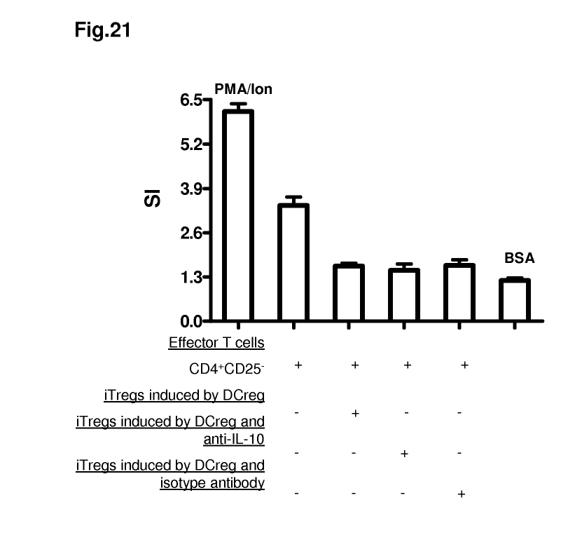

As reported, blockade of TGF-β and IL-10 could demolish the development and suppressive function of iTregs. In addition, the stage at which these cytokines exerted their effects was explored. iTregs with DCreg were induced, and TGF-β and IL-10 were blocked at different stages as shown in FIG. 6a. The roles of TGF-β and IL-10 were detected during the induction of iTregs by DCreg in vitro. GFP expression in CD4⁺CD25⁻ T cells was detected after 72 hrs of co-culture with CD11C⁺ DCreg 3 times each two days in the presence of anti-IL-10 or anti-TGF-β as stage 1 in FIG. 13A. These DCreg were pretreated with DNA and cogated protein for 48 hrs. As shown in FIG. 13B, blockade of TGF-β, but not IL-10 could decrease the generation of CD4⁺CD25⁻GFP⁺ iTregs. To confirm the crucial roles of TGF-b in Foxp3 induction, SB-525334, a potent TGFβ-receptor kinase inhibitor, was used to block the TGF-β signal pathway. As shown in FIG. 20, blockade of TGF-β receptor could decrease the iTreg induction. In order to detect suppressive function of iTregs when neutralizing IL-10, proliferation of responder T cells co-cultured with iTregs was induced in the present of anti-IL-10. Neutralization of IL-10 had no influence to iTregs function (FIG. 21).

Although TGF-β1 has been demonstrated to convert peripheral naive CD4⁺CD25⁻ T cells into CD4⁺CD25⁺ Tregs, its induction of the Foxp3 expression in CD4⁺CD25⁻ T cells alone is largely unknown. To investigate if TGF-β1 alone is able to induce the CD4⁺CD25⁻Foxp3⁺ iTregs in the presence of antigen stimulation, the CD4⁺CD25⁻ naive T cells isolated from Foxp3^{gfp} mouse were treated with anti-CD3 and anti-CD28 in the presence or absence of TGF-β1, respectively. As shown in FIG. 13C, the GFP expression was up-regulated in CD25⁻ T cells in the presence of TGF-β1 with a dose dependent manner. To test whether the IL-10 has a similar or synegestic effect with TGF-β1 on the Foxp3 expression, the IL-10 in the above system was added. As depicted in FIG. 13D, the IL-10 neither alone influenced the expression of Foxp3, nor had synergistic effects with TGF-β1. In conclusion, CD4⁺CD25⁻Foxp3⁺ iTregs were induced the TGF-β but not IL-10 secreted by dendritic cells directly.

### Example 13

### Autocrine IL-10 Modelate the Function of DCreg in Co-Immunization

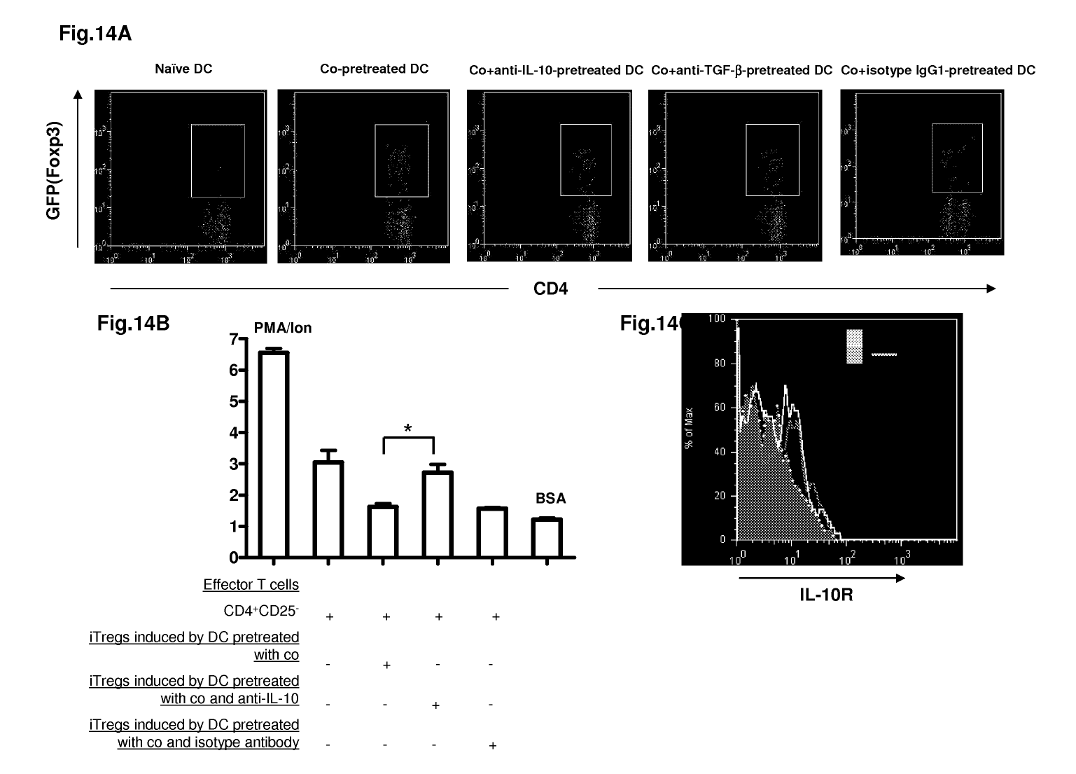

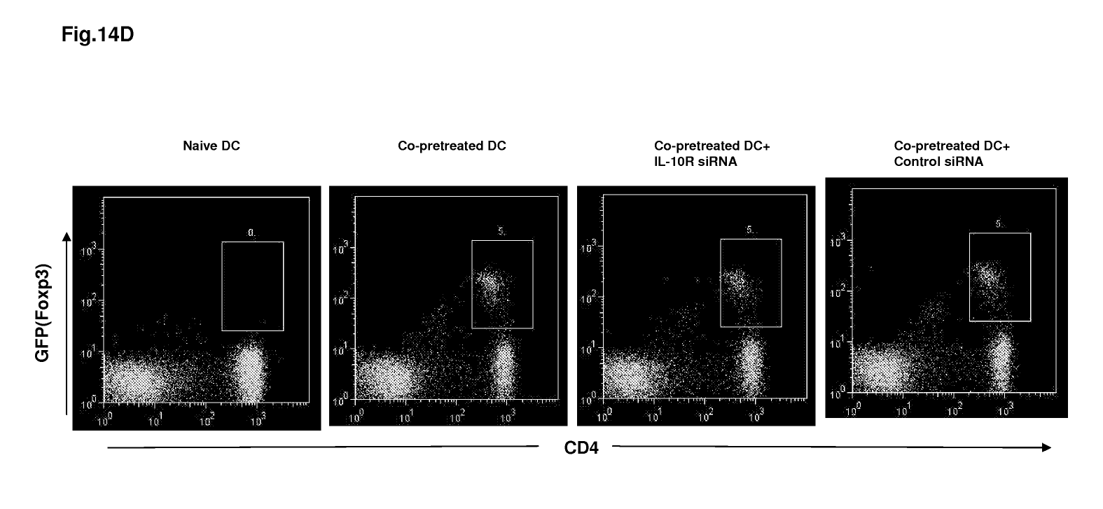

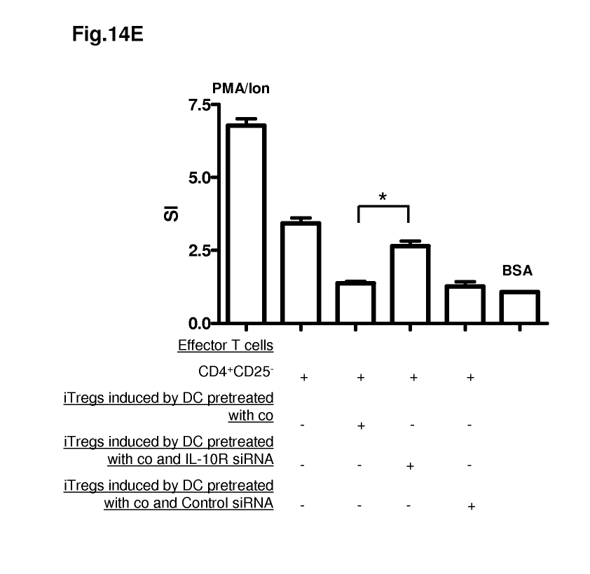

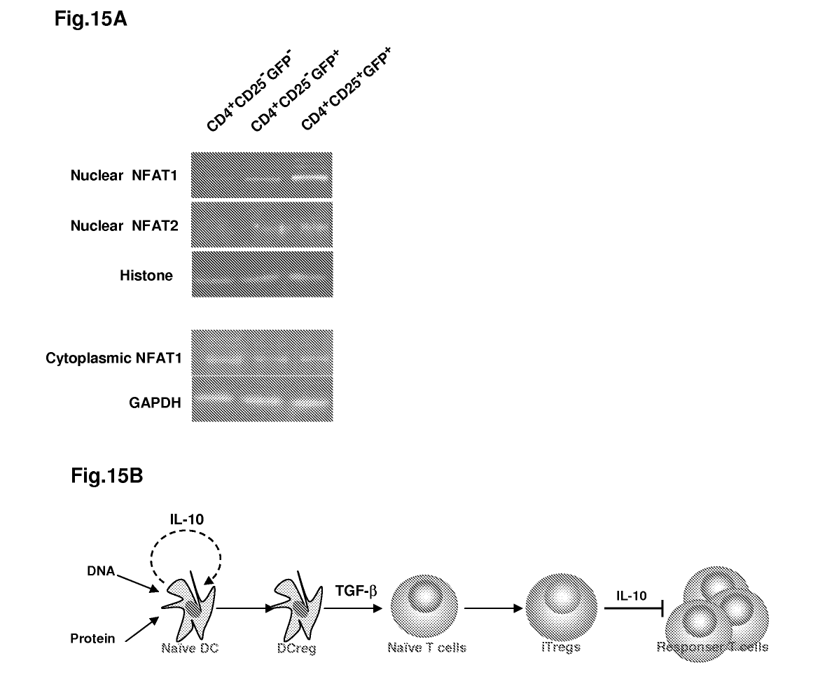

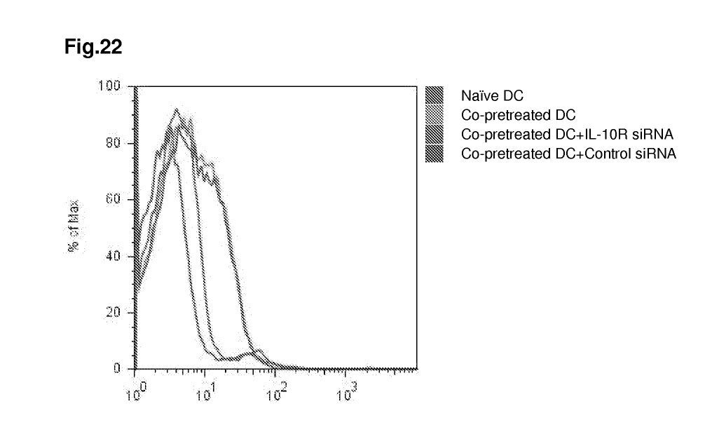

Based on the above results, IL-10 contributes to the induction of suppressive function of iTregs in co-immunization, but does not exert its effect directly on CD4+CD25− naive T cells. Accordingly, the relevance of autocrine IL-10 on DC functions was further examined. To do so, the ability of DCs pretreated with anti-IL-10 or anti-TGF-β to direct the differentiation of naive T cells was examined at stage 2, as shown in FIG. 13A. Naive CD11C⁺ dendritic cells were stimulated by Derp1 plasmid and recombined protein in the presence of anti-IL-10 or anti-TGF-β, and then added to these DCreg to the naive CD4⁺CD25⁻ T cells for 3 times. As shown in FIG. 14A, blocking neither endogenous IL-10 nor TGF-β could change the capacity of DCreg to induce CD4⁺CD25⁻Foxp3⁺ iTregs. The functional consequences of iTregs induced by different dendritic cell was tested by co-culture with responder T cells. From the results, it was found that the suppressive capacity of iTregs generated by dendritic cells pretreated with anti-IL-10 was decreased significantly (FIG. 14B). Autocrine IL-10 could up-regulate the IL-10R expression, and thus IL-10R expression was examined on different days after co-immunization. As shown in FIG. 14C, the results demonstrated that amounts of cell surface IL-10R was increased after co-immunization, and reached peak levels on day 3. To confirm the function of IL-10R, the function of iTreg induction was determined by dendritic cell knock-down of IL-10R via siRNA. The suppressive effect on expression of IL-10R was evaluated by FACS (FIG. 22). As shown in FIGS. 14D and E, in absence of IL-10R, dendritic cells decreased the capacity to enhance iTreg suppressive function, but did not influence on the Foxp3 induction. Binding of IL-10 to its receptor leads to the activation of JAK 1 and tyrosine kinase 2, and then to the recruitment and phosphorylation of STAT-1 and STAT-3. Western blot analysis of protein expression in Dcreg was performed, and it was found that phosphorylation of STAT-1 was inhibited after synchronous stimulation by DNA and protein, followed by down-regulation of CD40. In summary, autocrine IL-10 and IL-10R serve as a relevant modulatory loop for the development of DCreg.

This example demonstrates that the co-immunization with DNA and protein vaccine simultaneous induces a suppressive CD4 T cell subpopulation which exhibits a phenotype of CD4⁺CD25⁻Foxp3⁺. In HDM-induced allergic immune responses in lungs, the immunoregulatory effect of co-immunization was evaluated. The results indicate that co-immunization might not only contribute to significantly limit the inflammatory response in the lungs, but also to the inhibition of Th2 cytokines and the production of IgE.

Functionally, when co-cultured iTregs with CD4⁺CD25⁻ responder T cells, both of CD4⁺CD25⁻GFP⁺ and CD4⁺CD25⁺GFP⁺ Tregs can inhibit proliferation of the target T cells. This suppressive activity may be mainly attributed to the CD25⁻ subpopulation of energized cells, since the percent and Foxp3 expression of CD4⁺CD25⁺ T cells have no obvious up-regulation. In addition, blockade of CD4⁺CD25⁺ T cells with anti-CD25 mAb can not reverse the immuno toleration induced by co-immunization.

By FACS analysis, the iTregs were phenotyped as CD4⁺CD25⁻Foxp3⁺CTLA4⁻GITR⁻PD-1⁻. There was low expression of these well-known nTreg markers on the surface, indicating that the iTregs exerted their effect mainly via suppressive cytokines, but not cell-cell contact. To confirm this conclusion, iTregs and responder T cells were cultured in transwell plant, and IL-10 or TGF-β mAb was added. The results demonstrated that the suppressive function of iTregs were IL-10 independent.

Foxp3 regulates the expression of CD25 in mice via the formation of NFAT:Foxp3 complex bound to the promoters of the CD25, CTLA-4 and GITR target genes. In addition, ChIP analysis also shows that Foxp3 binding to IL-2R(CD25), CTLA-4, and other target genes in Tregs is stabilized when NFAT is activated. Therefore, it was hypothesized that the down-regulation of CD25, GITR and CTLA-4 is involved in NFAT1 diminishment in the presence of Foxp3. NFAT activation can be assessed as the nuclear translocation of NFAT. To test the hypothesis that NFAT activation is different in CD4⁺CD25⁻GFP⁺ and CD4⁺CD25⁺GFP⁺ T cells, immunoblotting analysis was performed in fractionated nuclear and cytoplasmic lysates from these cells. In the absence of stimulation, only low levels of nuclear NFAT1 were found in CD4⁺CD25⁻GFP⁻ and CD4⁺CD25⁻GFP⁺ T cells. In contrast, higher level of nuclear NFAT1 was detected in CD4⁺CD25⁺GFP⁺ nTregs. Correspondingly, a lower level of NFAT1 was seen in the cytoplasmic fraction in CD4⁺CD25⁺GFP⁺ than in the CD4⁺CD25⁻GFP⁺ and CD4⁺CD25⁻GFP⁻ T cells (FIG. 14D), suggesting that NFAT1 is being held in its inactive state in T cells or CD4⁺CD25⁻GFP⁺ iTregs. On other hand, the Foxp3 expression was induced through the cooperation of Smad3 and NFAT2 in CD4⁺CD25⁺ nTreg development. Accordingly the level of nuclear NFAT2 was analyzed. As expected, the level of NFAT2 was detectable in the nuclear fraction from CD4⁺GFP⁺ T cells, no matter whether CD25 was expressed. The NFAT2 in cytoplasmic lysates could not be detected in all three subtypes of T cells. Collectively, these data illustrate differential regulation of NFAT activation in CD4⁺CD25⁻Foxp3⁺ iTregs compared with CD4⁺CD25 Foxp3⁺ nTregs and CD25⁻ Th cells.

The results described above illustrate that TGF-β1 contributes to Foxp3 expression in CD4⁺CD25⁻ T cells in co-immunization. The generation of iTreg as affected when in the presence of anti-TGF-β1-neutralizing antibody. TGF-β1 was blocked at different stage during the initiation of iTregs induced by DCreg in vitro. The results demonstrate that DC-secreting TGF-β1 induce CD4⁺CD25−Foxp3⁺ iTregs directly. In addition, TGF-β1 also can induce Foxp3 expression in CD4⁺CD25⁻ T cells alone under conditions involving anti-CD3 and anti-CD28 stimulation. Unlike TGF-β1, IL-10 fails to induce Foxp3 in CD4⁺CD25⁻ T cells, but blockade of IL-10 could demolished the suppressive function of iTregs. The results demonstrate that IL-10 contributes to the initiation of suppressive ability of iTregs. Autocrine IL-10 impairs dendritic cell DC-derived immune responses. The IL-10 effect was blocked on the naive T cells and DC respectively. The results show that IL-10 contributes to the induction of immature dendritic cells, and then strengthens the suppressive capacity of iTregs, but does not directly effect iTregs.

In summary, this example demonstrates that the co-immunization protocol with Der-p1 DNA vaccine and its cognate-recombined protein induces CD4⁺CD25⁻Foxp3⁺ iTregs. Both TGF-β1 and IL-10 are critical factors in the development of these iTregs in co-immunization. Additionally, TGF-β1 and IL-10 exert their effects in development and suppressive function of CD4⁺CD25⁻Foxp3⁺ iTregs. Since co-immunization induces CD4⁺CD25⁻Foxp3⁺ iTregs via TGF-β1 and IL-10, this discloses novel, therapeutic strategies for the treatment of autoimmune, chronic inflammatory and allergic diseases.

### Example 14

### Materials and Methods for Examples 15-19

### Mice and Reagents

Female BALB/c and C57BL/6 mice (8-10 wk of age) were from the Animal Institute of Chinese Medical Academy (Beijing, China). All animals received pathogen-free water and food.

Flexset IL-10 and fluorescently labeled anti-mouse monoclonal antibodies including anti-IL-10-phycoerythrin (PE), anti-FoxP3-allophycocyanin (APC), anti-IL-10-APC, anti-CD40-APC, anti-CD11c-APC, anti-CD11c-fluoroscein isothiocyanate (FITC), anti-CD40-PE and isotype controls were purchased from BD Biosciences (San Diego, Calif., USA). Alexa Fluor 546 (AF)-labeled goat anti-rabbit IgG was purchased from Invitrogen (Carlsbad, Calif., USA). Carboxyfluorescein succinimidyl ester (CFSE) was obtained from Molecular Probes (Eugene, Oreg., USA). Antibodies against IRAK-1, caveolin-1, phospho-caveolin-1Tyr14, Tollip, SOCS-1, NF-κB p65, phospho-NF-κB p65^{Ser536}, STAT-1α, phospho-STAT-1α^{Tyr701} and -STAT-1α^{Ser727}, CD40, GAPDH, and histone were purchased from Santa Cruz Biotechnology (Santa Cruz, Calif., USA). E. coli LPS, 5-(N,N-Dimethyl) amiloride hydrochloride, monodansylcadaverine (MDC) and filipin were purchased from Sigma-Aldrich (St. Louis, Mo., USA).

Vaccine Preparations

The DNA vaccines, pVAX-OVA (designated as pOVA) and pVAX-OVA323 (designated as pOVA323) were obtained by inserting the encoding DNA sequence for the whole hen ovalbumin protein (OVA) or its dominant epitope (at the aa323-339 region) into pVAX1 (Invitrogen Inc., Carlsbad, Calif., USA), at Xba I and Hind III sites by digestions, respectively. The reverse strand of OVA coding sequence was cloned into pVAX and yielded a non-expressing pVAX-OVArev (designated as pOVArev). pcD-mZP3 encoding mouse zona pullucida 3 (ZP3) and mZP3 recombinant protein expressed in E. coli were prepared and described in our previous report 13. OVA was purchased from Sigma-Aldrich and the OVA peptide (aa323-339, named as OVA₃₂₃) or FITC-labeled OVA₃₂₃ were synthesized by GL Biochem Co., Ltd. (Shanghai, China). All plasmids were purified to remove the endotoxin by EndoFree Plasmid Maxi Kit (QIAGEN, Tokyo, Japan) and used as the DNA vaccines by dissolving in PBS at 2 mg/ml. Recombinant proteins and peptides were dissolved in PBS at 2 mg/ml and sterilized by filtration.

Culture and Stimulation of Jaws II Dendritic Cells

The JAWS II mouse DC line was purchased from the American Type Culture Collection (ATCC, Manassas, Va., USA) and maintained in complete growth medium containing minimum essential medium (MEM) alpha with ribonucleosides, deoxyribonucleosides, 4 mM L-glutamine, and 1 mM sodium pyruvate (Invitrogen Inc., Carlsbad, Calif., USA), and supplemented with 20% fetal bovine serum (ATCC) and 5 ng/ml murine recombinant GM-CSF (R&D Systems, Inc., Minneapolis, Minn., USA). The cells were incubated at 37° C. with 5% CO2 and treated with different antigens (10 μg/ml) such as pVAX, pOVA, OVA, pOVA323 and OVA323 for 24 h. For inhibitor treatment, JAWS II cells were pre-treated with filipin (10 μg/ml), MDC (50 μM) for 30 min at 37° C., respectively, or with amiloride (5 mM) for 10 min at 37° C., and washed with medium, then stimulated with antigens.

Silencing of Cav-1 and Tollip in JAWS II and Treatment by DNA and Protein

Wild type (WT), or Cav-1- and/or Tollip-deficient DCs were co-treated with 10 μg/ml pOVA₃₂₃ and OVA₃₂₃ or pVAX and OVA₃₂₃ for 24 h. For in vitro function of DCregs, CD4+ T cells were purified from the spleen of mice immunized with OVA in incomplete Freund's adjuvant (IFA) and labeled with CFSE. CFSE-CD4+ T cells co-cultured with co-treated DCs for 5 d and then T cell proliferation and expression of Foxp3 and IL-10 were detected. For in vivo function of DCregs, 2×10⁶ co-treated DCs were transferred into syngeneic C57BL/6 mice and these mice were immunized with OVA in IFA on days 0 and 7. On day 14, mice were injected with 25 μg OVA into a footpad to test for delayed-type hypersensitivity (DTH) response. On day 15, mice were sacrificed to detect T cell proliferation and expression of Foxp3 and IL-10.

Semi-Quantitative RT-PCR Analysis for Cytokines

Total RNA was isolated from about 5×10⁶ cells using the TRIzol reagent (Promega, Wis., USA). The amount of cytokine-specific mRNA was determined by semi-quantitative reverse transcription-polymerase chain reaction (RT-PCR). Primers for hypoxanthine phosphoribosyl transferase (HPRT), a housekeeping gene, or for cytokine genes were used. The sequences of the primers and conditions for PCRs are listed in Table S1.

Western Blotting

Protein samples were separated by 10% sodium dodecyl sulfate-polyacrylamide gel electrophoresis (SDS-PAGE), followed by transfer onto a nitrocellulose membrane and detection with specific antibodies and an anti-actin Ab serving as a reference for sample loading. For detection of NF-κB, cytoplasmic and nuclear proteins were extracted. Nuclear and cytoplasmic extracts were analyzed by immunoblotting. The ECL (GE Healthcare Europe, Uppsala, Sweden) method was used for protein detection.

Induction of Inflammatory Bronchitis and Autoimmune Ovarian Disease (AOD) in Mice

Inflammatory bronchitis was induced in BALB/c mice as previously described and with some modifications. In brief, Mice were injected intraperitoneally with 100 μg OVA (0.1 ml of 1 mg/ml OVA/alum complexes in PBS) on days 0 and 7. This was followed by intra-tracheally delivery of 100 μg (100 μl of 1 mg/ml) OVA to each animal on days. Control mice received PBS. AOD was induced in C57BL/6 mice as previously described 11.

Histology Analysis

Lung or ovary were fixed in 4% paraformaldehyde or Bouin's solution and embedded in paraffin blocks. Sections were cut and stained with hematoxylin and eosin (H&E). Histopathology of lung or ovary was evaluated under a light microscope.

Flow Cytometric (FACS) Analysis

DCs or T cells were stained with the appropriate PE, FITC or APC-conjugated mAbs in PBS for 30 min at 4° C., according to previous studies. The cells were analyzed with FlowJo.

A multiplexed flow cytometric assay (the Th1/Th2 cytokine CBA kit, BD Biosciences) was used to test the production of tumor necrosis factor (TNF)-γ, IL-4, IL-5 and interferon (IFN)-γ in serum of immunized mice.

Statistics

Student's t test was used for data analysis. Differences were considered to be statistically significant if p<0.05.

### Example 15

### CD40^{low} is a Marker for Co-Immunization-Induced DCregs

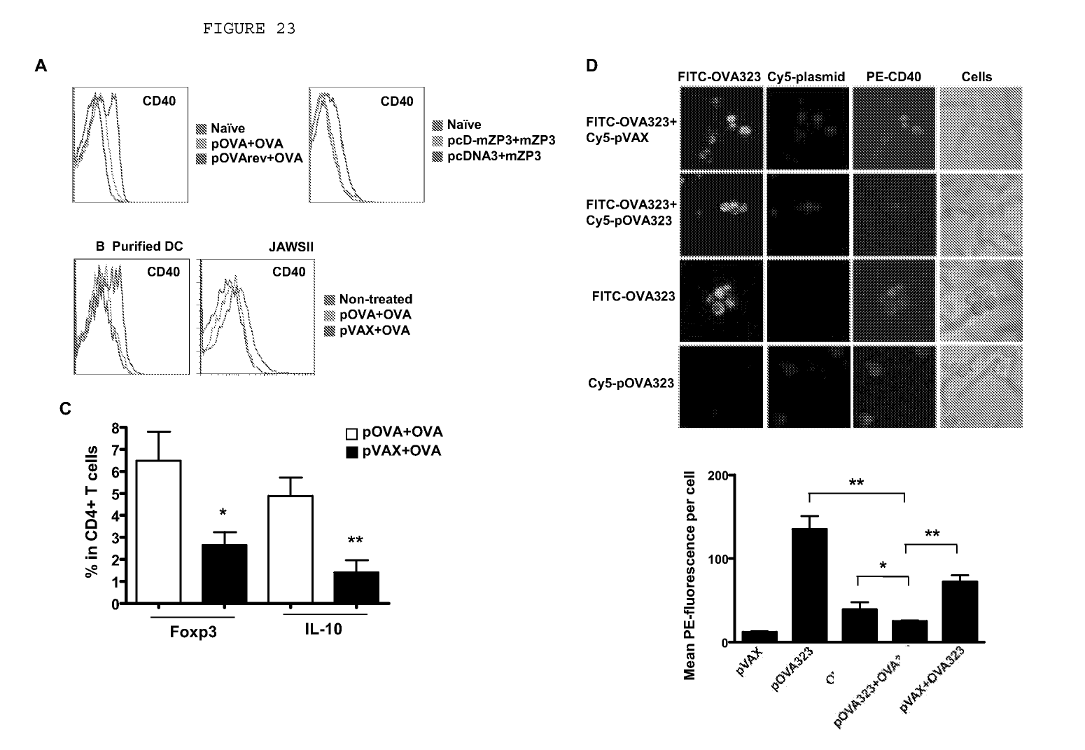

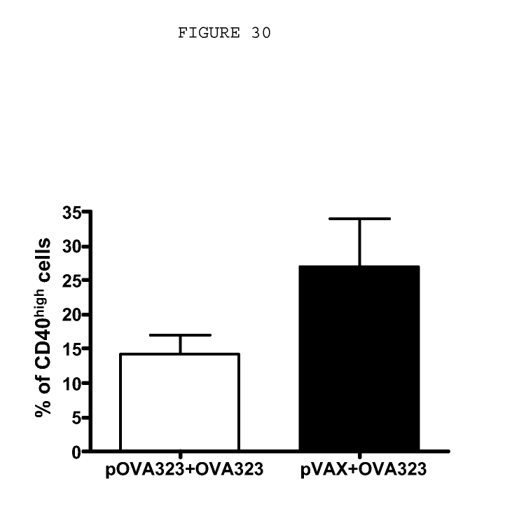

CD11c+CD40_{low}IL-10high DCregs were demonstrated to be induced in vivo after co-administration of sequence-matched DNA and protein immunogens. To test whether the low CD40 expression is a reliable phenotype of co-immunization-induced DCregs, a eukaryotic expression construct encoding the full-length hen ovalbumin (pOVA) was constructed and used in combination with the protein (OVA). pOVA and OVA were co-injected intramuscularly into one group of mice (pOVA+OVA). As a control for gene-specificity, a DNA construct containing the noncoding strand of OVA (pOVArev) and OVA were co-injected into another group of mice (pOVArev+OVA). On day 2, DCs were isolated from both groups, together with a group of non-injected mice (naive), and compared their expression of CD40 by FACS. Expression of CD40 in the pOVA+OVA group was higher than that in the naïve group, but lower than that in the pOVArev+OVA group (FIG. 30A), confirming the CD40low phenotype. We also tested an additional combination of DNA and protein immunogens, comprised of a DNA construct coding for the murine ZP3 and the ZP3 protein, and observed a similar result (FIG. 23A). These results suggest that the low CD40 expression is a consistent phenotype induced by co-administration of sequence-matched DNA and protein immunogens.

The experiment was repeated in culture with primary DCs and the DC line JAWS II. pOVA and OVA, or pVAX and OVA (control), were added directly to freshly isolated CD11c+ cells and JAWS II cells for 24 h. The result showed that, in both cell types, CD40 expression was lower following the pOVA+OVA treatment than following the control treatment (FIG. 23B), suggesting that the CD40low phenotype can also be induced in vitro in cultured primary DCs and DC lines.

Our previous studies showed that DCregs induced in vivo by co-immunization could convert naïve T cells into Tregs in vivo and in vitro. To determine whether the in vitro induced CD40^{low} DCs could do the same, we tested the activity of CD40^{low} JAWS II cells by co-culturing them with CFSE-labeled syngeneic CD4+ T cells from OVA-sensitized. The expressions of Foxp3 and IL-10 within the CFSE+ cells were analyzed after 5 d co-culture. The result showed that the CD40^{low} JAWS II cells caused expansion of Foxp3+ and IL-10+ T cells (FIG. 23C), confirming that the CD40^{low} DCs generated in vitro were in fact DCregs.

Because the appearance of the CD40¹′ phenotype required matching sequence between DNA and protein, it might require uptake of both DNA and protein by the same DC. To test this hypothesis, pOVA₃₂₃ (a DNA construct encoding the OVA₃₂₃₋₃₃₉ dominant epitope) and pVAX (the empty vector) were labeled with Cy5 and OVA₃₂₃ (the OVA₃₂₃₋₃₃₉ peptide) with FITC. As depicted in FIG. 23D, the low expression of CD40 was observed only in individual DCs taking up both Cy5-pOVA₃₂₃ and FITC-OVA₃₂₃, as observed by confocal microscopy. Taken together, these results suggest that CD40^{low} is a reliable marker for DCregs generated by co-immunization because the display of this maker requires co-uptake of sequence-matched DNA and protein immunogens.

### Example 16

### DCs Co-Uptake DNA and Protein Immunogens Via Clathrin- and Caveolae-Mediated Endocytosis

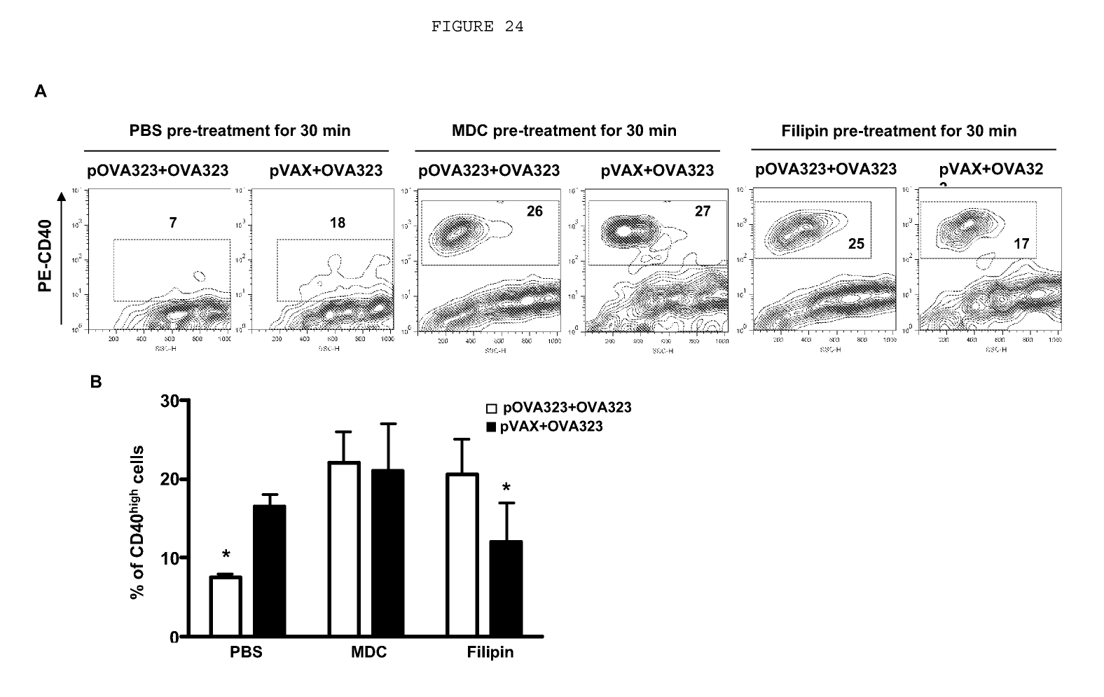

DCs take up exogenous antigens via various mechanisms including clathrin-mediated endocytosis, caveolae-mediated endocytosis, and macropinocytosis. To define which pathway(s) were involved in the co-uptake of DNA and protein immunogens, JAWS II cells were pretreated with MDC, a specific inhibitor of clathrin formation, or filipin, an inhibitor of caveolae trafficking, before being treated with pOVA₃₂₃+OVA₃₂₃. Using CD40^{low} as a marker, and although both MDC and filipin could prevent the CD40¹′ phenotype, filipin was more effective than MDC. This suggests that the CD40^{low} phenotype is primarily the result of caveolae-mediated endocytosis (FIGS. 24, A & B). Another inhibitor, amiloride, an inhibitor for macropinocytosis, had no effect on CD40 expression (FIG. 30).

### Example 17

### Co-Immunization Down-Regulates NF-κB and STAT-1a by Activating Negative Signaling Pathways

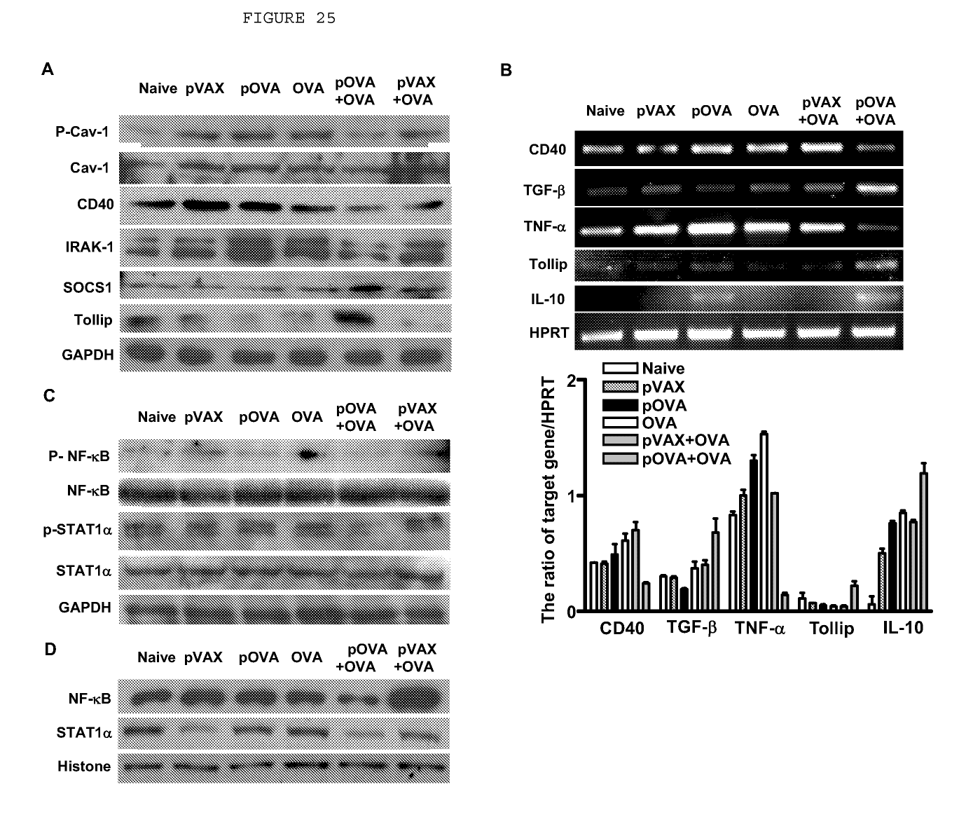

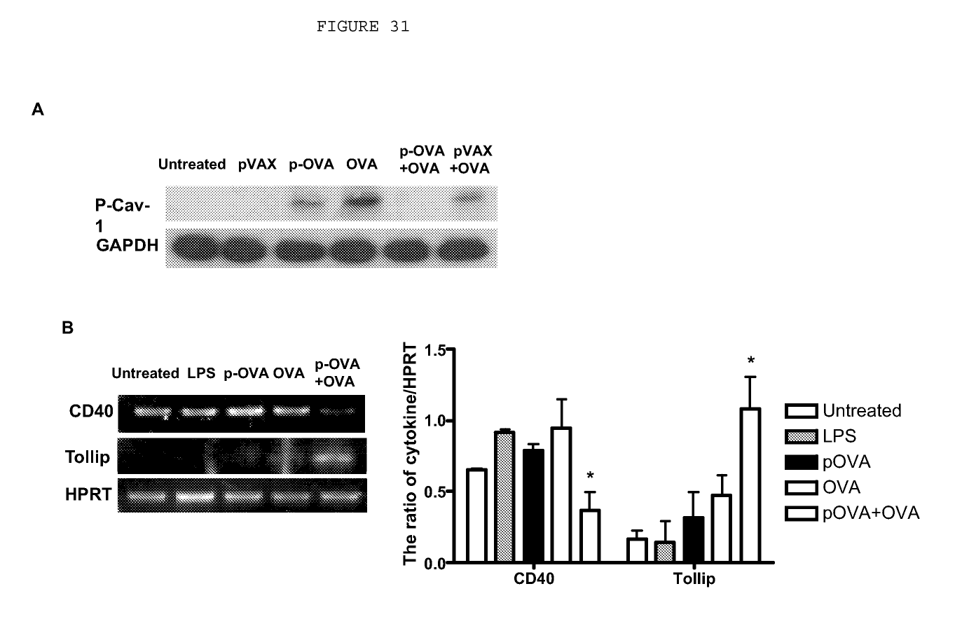

The transcription factor NF-κB regulates the expression of CD40 23,24 and IRAK-1 regulates the activation of NF-κB. Interestingly, caveolin-1 (Cav-1), a component of caveolae, was previously shown to form complex with Tollip to suppress IRAK-1's kinase activity under the steady-state condition. Phosphorylation of Cav-1Tyr14 was strongly inhibited in spleen DCs isolated from mice treated with pOVA+OVA, as compared to those isolated from mice treated with pOVA, OVA, or pVAX+OVA (FIG. 25A). Lack of phosphorylated Cav-1 was also seen in JAWS II cells fed pOVA+OVA in culture (FIG. 31A).

Following that lead, we investigated the expression of Tollip and the activation of IRAK-1 in spleen DCs in response to pOVA+OVA co-immunization. We observed that the transcription of Tollip, and TGF-γ and IL-10 as well, was up-regulated in co-immunized mice; whereas the transcription of CD40 and TNF-γ was down-regulated (FIG. 25B). Similar results were also observed in JAWS II cells fed pOVA+OVA in culture (FIG. 31B). Phosphorylation of IRAK-1 was also significantly inhibited in co-immunized mice (FIG. 25A), which agrees well with inhibited Cav-1 phosphorylation and increase of Tollip.

Because SOCS negatively regulates the activation of IRAKs and the JAK-STAT pathway, the level of the SOCS1 protein was analyzed. SOCS1 was significantly increased in response to pOVA+OVA co-immunization (FIG. 25A). Together, these results indicate that co-immunization alters phosphorylation of Cav-1 and expression of Tollip and SOCS1 to activate negative signaling.

Next, the activation of the transcription factors NF-κB and STAT-1α was analyzed. The phosphorylation of NF-κB p65^{Ser536} and STAT-1α^{Tyr701} was strongly inhibibited in pOVA+OVA co-immunized mice (FIG. 25C). The translocation of NF-κB and STAT-1a were also inhibited since the concentration of NF-κB p65 and STAT-1α in nuclear was decreased in the co-immunized group (FIG. 25D), suggesting down-regulated activation of NF-κB and STAT-1α after the co-immunization.

Taken together, these results demonstrate that co-immunization activates negative pathways mediated by Cav-1, leading to down-regulation of the activity of NF-κB and STAT-1α and reduced expression of CD40.

### Example 18

### Silencing Cav-1 and Tollip Prevents the Induction of DCregs

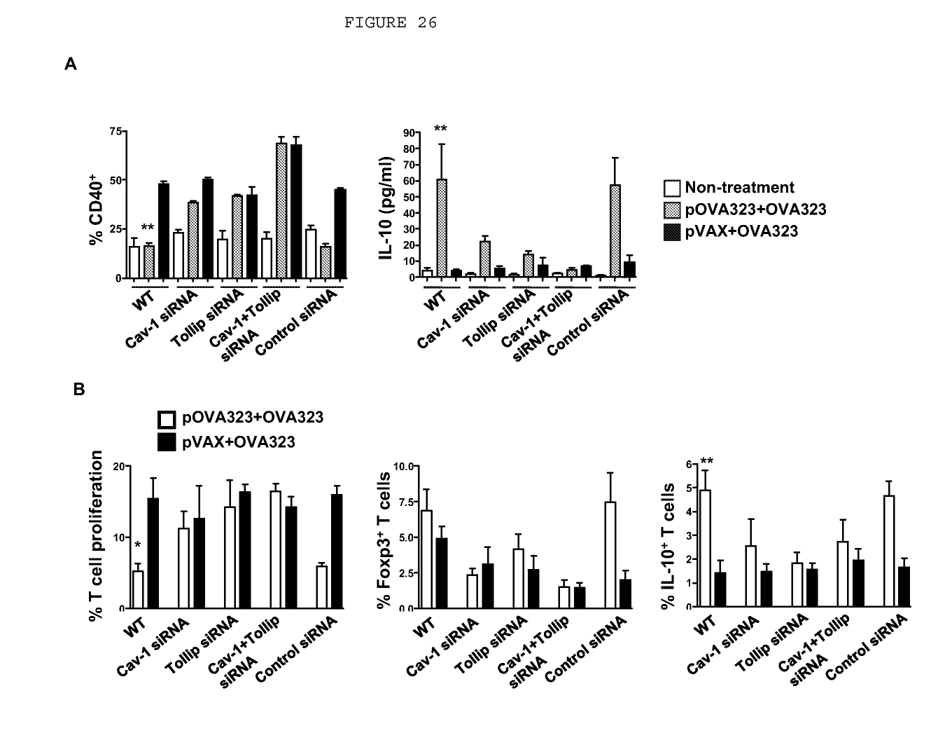

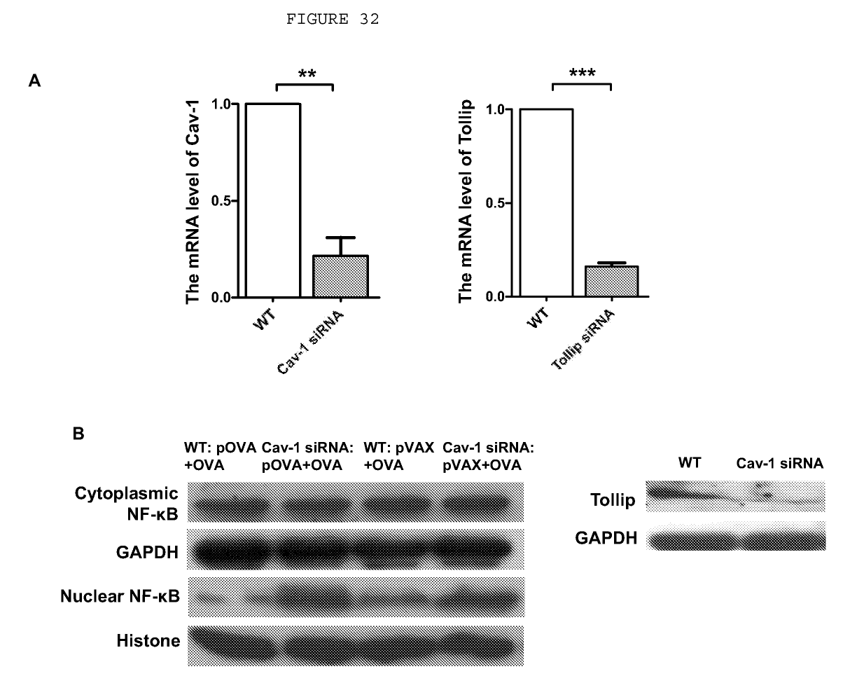

In order to address the role of Cav-1 and Tollip in the induction of DCregs, we used RNA interference (RNAi) to silence the expression of Cav-1 and Tollip. The efficiency of RNAi reached approximately 80% for both genes in JAWS II cells (FIG. 32a). Silencing of both Cav-1 and Tollip completely prevented JAWS II cells from differentiating into DCregs when fed pOVA323+OVA323, as judged by the increased CD40 expression and decreased IL-10 production following silencing, whereas silencing of either Cav-1 or Tollip alone was partially effective (FIG. 26a). Further, translocation of NF-κB was increased and the production of Tollip was decreased following Cav-1 silencing (FIG. 32b).

Functionally, Cav-1- and/or Tollip-deficient and pOVA323+OVA323 treated JAWS II cells were unable to suppress the proliferation of responder T cells in a co-culture assay, or induce iTreg conversion or IL-10 expression (FIG. 26b). These data show that both Cav-1 and Tollip play a critical role in the induction of DCreg phenotype and function following co-immunization.

### Example 19

### Cav-1- and/or Tollip-Deficient DCs are not Tolerogenic In Vivo

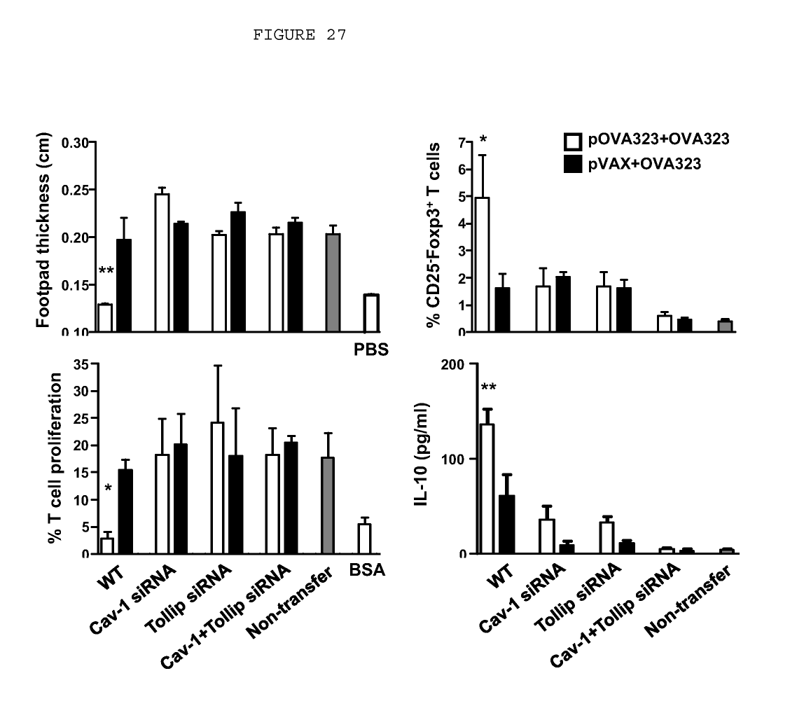

To determine if the Cav-1- and/or Tollip-deficient JAWS II cells had also lost their ability to promote tolerance in vivo, we transferred them into syngeneic mice after treating them with pOVA323+OVA323. The recipient mice were then challenged by immunization with OVA in IFA. While control mice transferred with pOVA323+OVA323 treated wild-type JAWS II cells inhibited the induction of DTH and OVA-reactive T cells and increased the expression of Foxp3 and production of IL-10 in CD4+CD25− T cells (CD25−iTreg), the silenced JAWS II failed to the same (FIG. 27). This result confirms that the silenced JAWS II cells are not tolerogenic.

### Example 20

### Co-Immunization-Induced DCregs Ameliorate Inflammatory Bronchitis and Autoimmune Ovarian Disease in Mice

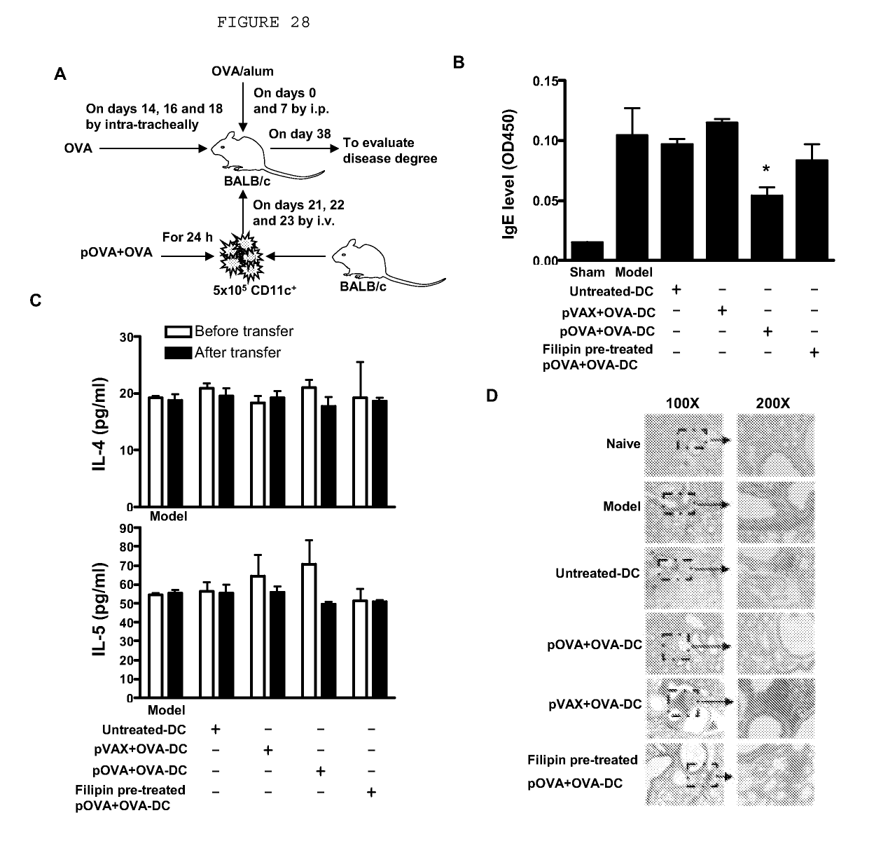

To assess the potential of co-immunization-induced DCregs as a therapeutic for inflammatory and autoimmune disease, we fed cultured primary DCs pOVA+OVA and used the resulting DCregs to treat BALB/c mice with OVA-induced inflammatory bronchitis (FIG. 28A). Adoptive transfer of the DCregs significantly decreased the level of IgE in recipient mice (FIG. 28B). The levels of IL-4 and IL-5 were also reduced in recipient mice, although they did not reach the statistical significance (FIG. 28C). Histological analysis of lung sections from the mice revealed a nearly normal lung morphology that was free of cell infiltration (FIG. 28D). As expected, the anti-inflammatory effect of the pOVA+OVA treated DCs was absent if the DCs were pretreated with filipin.

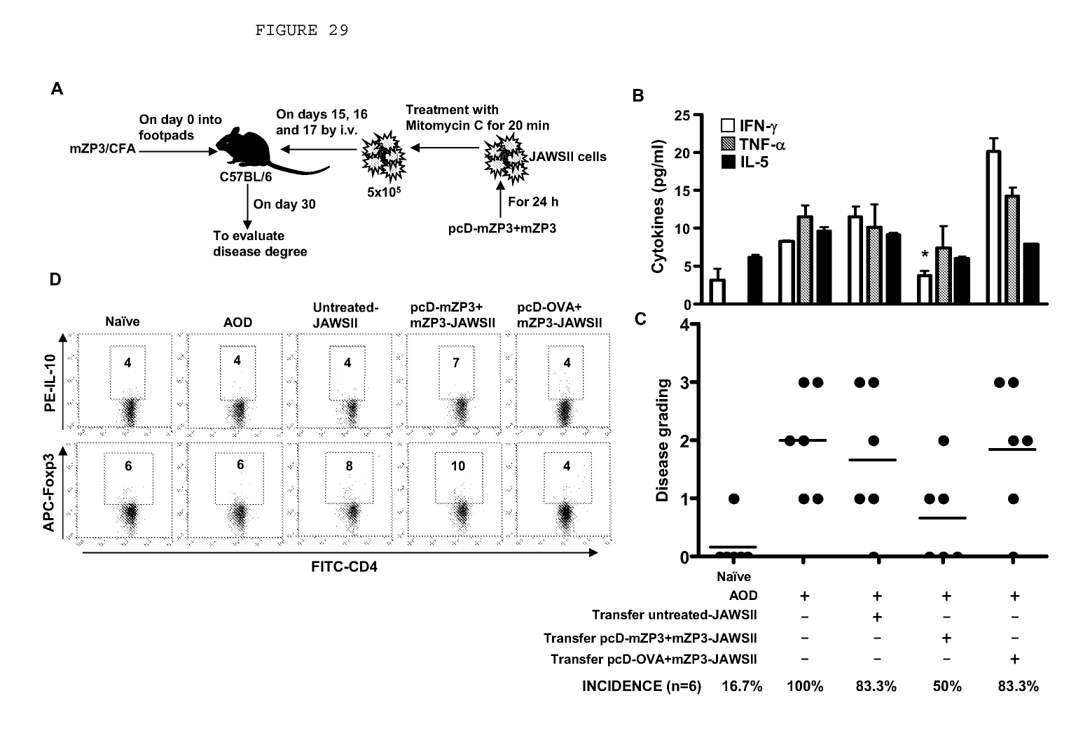

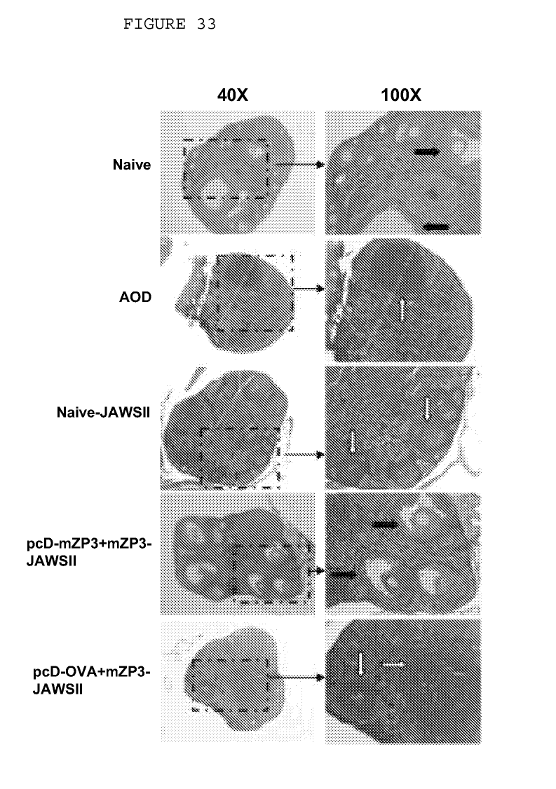

To determine whether a similar therapeutic effect could reproduce with a DC line, we fed cultured JAWS II cells pcD-mZP3, a DNA construct encoding the mouse ZP3 protein, and the mZP3 protein (pcD-mZP3+mZP3). The resulting DCregs were adoptively transferred into C57BL/6 mice with mZP3-induced autoimmune ovarian disease (AOD) (FIG. 29A). Subsequently, we observed reduced production of IFN-γ, IL-5, and TNF-α (FIG. 29B) and reduced severity of AOD (FIG. 29C) in the recipient mice. Histological analysis of ovarian sections revealed a nearly normal histological structure without notable cell infiltration (FIG. 33). FACS analysis of the spleen further showed increased frequency of IL-10+ and Foxp3+CD4+ T cells (FIG. 29D). Taken together, these results suggest that DCregs generated in culture by feeding primary DCs or DC lines sequence-matched DNA and protein immunogens are potentially useful for adoptive immunotherapy.

We claim:

1. A vaccine capable of suppressing an autoimmune disease comprising an antigenic peptide and a DNA encoding the peptide, wherein the antigenic peptide/DNA stimulate iTreg cells.

2. The vaccine of claim 1, wherein the antigen is associated with a condition selected from the group consisting of allergy, asthma, and autoimmune disease.

3. The vaccine of claim 2, wherein the antigen is associated with allergy or asthma and is selected from the group consisting of dermatophagoides pteronyssinus 1 peptide, a fragment thereof, and a variant thereof.

4. The vaccine of claim 2, wherein the antigen is associated with an autoimmune disease and is selected from the group consisting of insulin peptide, myelin oligodendrocyte glycoprotein, myelin basic protein, and oligodendrocyte-specific protein, zonapellucida protein peptide, dermatophagoides pteronyssinus 1 peptide, α-myosin peptide, coxsackievirus B4 structural protein peptide, group A streptococcal M5 protein peptide, (Q/R)(K/R) RAA, type II collagen peptide, thyroid peroxidase, thyroglobulin, pendrin peptide, acetylcholine receptor peptide, human S-antigen, a fragment thereof, and a variant thereof.

5. The vaccine of claim 1, wherein a vector comprises the DNA.

6. The vaccine of claim 5, wherein the vector is selected from the group consisting of pVAX, pcDNA3.0, and provax.

7. The vaccine of claim 6, wherein the vector and antigenic peptide are at a mass ratio selected from the group consisting of 5:1 and 1:5; and 1:1 and 2:1.

8. A vaccination kit comprising a vaccine administration device and the vaccine of claim 1.

9. The kit of claim 8, wherein the vaccine administration device is selected from the group consisting of vaccine gun, needle, and an electroporation device.

10. A method for treating an autoimmune disease comprising administering to a patient in need thereof the vaccine of claim 1.

11. The method of claim 10, wherein the autoimmune disease is type I diabetes mellitus.

12. The method of claim 11, wherein the antigen is selected from the group consisting of insulin peptide, a fragment thereof, or a variant thereof.

13. The method of claim 10, wherein the autoimmune disease is multiple sclerosis.

14. The method of claim 13, wherein the antigen is selected from the group consisting of myelin oligodendrocyte glycoprotein, myelin basic protein, and oligodendrocyte-specific protein.

15. The method of claim 10, wherein the autoimmune disease is autoimmune ovarian disease.

16. The method of claim 15, wherein the antigen is selected from the group consisting of zonapellucida protein peptide, a fragment thereof, and a variant thereof.

17. The method of claim 10, wherein the autoimmune disease is a dust mite allergy.

18. The method of claim 17, wherein the antigen is selected from the group consisting of dermatophagoides pteronyssinus 1 peptide, a fragment thereof, and a variant thereof.

19. The method of claim 10, wherein the autoimmune disease is myocarditis.

20. The method of claim 19, wherein the antigen is selected from the group consisting of α-myosin peptide, coxsackievirus B4 structural protein peptide, group A streptococcal M5 protein peptide, fragments thereof, and variants thereof.

21. The method of claim 10, wherein the autoimmune disease is rheumatoid arthritis.

22. The method of claim 21, wherein the antigen is selected from the group consisting of peptide (Q/R)(K/R) RAA, type II collagen peptide, fragments thereof, and variants thereof.

23. The method of claim 10, wherein the autoimmune disease is thyroiditis.

24. The method of claim 23, wherein the antigen is selected from the group consisting of thyroid peroxidase, thyroglobulin, pendrin peptide, fragments thereof, and variants thereof.

25. The method of claim 10, wherein the autoimmune disease is myasthenia gravis.

26. The method of claim 25, wherein the antigen is selected from the group consisting of acetylcholine receptor peptide, fragments thereof, and variants thereof.

27. The method of claim 10, wherein the autoimmune disease is autoimmune uveitis.

28. The method of claim 27, wherein the antigen is selected from the group consisting of human S-antigen, fragments thereof, and variants thereof.

29. The method of claim 10, wherein the autoimmune disease is asthma.

30. The method of claim 29, wherein the antigen is selected from the group consisting of dermatophagoides pteronyssinus 1 peptide, fragments thereof, and variants thereof.


SEQUENCE LISTING (192 sequences)

SEQ ID NO: 1 - protein, 15 aa, Pulex irritans
QEKEKCMKFCKKVCK

SEQ ID NO: 2 - protein, 14 aa, Pulex irritans
PDWKVSKECKDPNN

SEQ ID NO: 3 - protein, 320 aa, Dermatophagoides pternyssinus
MKIVLAIASLLALSAVYARPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 4 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 5 - protein, 11 aa, Homo sapiens
MRLLPLLALLA

SEQ ID NO: 6 - protein, 9 aa, Homo sapiens
LYLVCGERG

SEQ ID NO: 7 - protein, 15 aa, Homo sapiens
GIVEQCCTSICSLYQ

SEQ ID NO: 8 - protein, 13 aa, Homo sapiens
QHLQKDYRAYYTF

SEQ ID NO: 9 - protein, 13 aa, Homo sapiens
YTFLNFMSNVGDP

SEQ ID NO: 10 - protein, 13 aa, Homo sapiens
RVLNIDLLWSVPI

SEQ ID NO: 11 - protein, 13 aa, Homo sapiens
DWIHIDTTPFAGL

SEQ ID NO: 12 - protein, 13 aa, Homo sapiens
KGLGVDLLWTLEK

SEQ ID NO: 13 - protein, 13 aa, Homo sapiens
EWVHIDTTPFASL

SEQ ID NO: 14 - protein, 13 aa, Homo sapiens
FTLGLDLSWSISL

SEQ ID NO: 15 - protein, 13 aa, Homo sapiens
EWIHVDSRPFASL

SEQ ID NO: 16 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 17 - protein, 22 aa, Homo sapiens
MASLSRPSLPSCLCSFLLLLLL

SEQ ID NO: 18 - protein, 23 aa, Homo sapiens
VIGPRHPIRALVGDEVELPCRIS

SEQ ID NO: 19 - protein, 33 aa, Homo sapiens
MEVGWYRPPFSRVVHLYRNGKDQDGDQAPEYRG

SEQ ID NO: 20 - protein, 197 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 21 - protein, 23 aa, Homo sapiens
GRSPLPSHARSQPGLCNMYKDSH

SEQ ID NO: 22 - protein, 15 aa, Homo sapiens
KDSHHPARTAHYGSL

SEQ ID NO: 23 - protein, 20 aa, Homo sapiens
DSHHPARTAHYGSLPQKSHG

SEQ ID NO: 24 - protein, 26 aa, Homo sapiens
WGAEGQRPGFGYGGRASDYKSAHKGF

SEQ ID NO: 25 - protein, 15 aa, Homo sapiens
MYKDSHHPARTAHYG

SEQ ID NO: 26 - protein, 12 aa, Homo sapiens
KDSHHPARTAHY

SEQ ID NO: 27 - protein, 274 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 28 - protein, 19 aa, Homo sapiens
LFCGCGHEALTGTEKLIET

SEQ ID NO: 29 - protein, 11 aa, Homo sapiens
VATGLCFFGVA

SEQ ID NO: 30 - protein, 21 aa, Homo sapiens
LTGTEKLIETYFSKNYQDYEY

SEQ ID NO: 31 - protein, 20 aa, Homo sapiens
TCQSIAFPSKTSASIGSLCA

SEQ ID NO: 32 - protein, 16 aa, Homo sapiens
IAFPSKTSASIGSLCA

SEQ ID NO: 33 - protein, 20 aa, Homo sapiens
TSASIGSLCADARMYGVLPW

SEQ ID NO: 34 - protein, 81 aa, Homo sapiens
MSQKPAKEGPRLSKNQKYSEHFSIHCCPPFTFLNSKKEIVDRKYSICKSGCFYQKKEEDWICCACQKTRLKRKIRPTPKKK

SEQ ID NO: 35 - protein, 218 aa, Homo sapiens
MVATCLQVVGFVTSFVGWIGVIVTTSTNDWVVTCGYTIPTCRKLDELGSKGLWADCVMATGLYHCKPLVDILILPGYVQACRALMIAASVLGLPAILLLLTVLPCIRMGQEPGVAKYRRAQLAGVLLILLALCALVATIWFPVCAHRETTIVSFGYSLYAGWIGAVLCLVGGCVILCCAGDAQAFGENVSTTLRALAPRLMRRVPTYKRAARLPTEVL

SEQ ID NO: 36 - protein, 14 aa, Homo sapiens
HPIRALVGDEVELP

SEQ ID NO: 37 - protein, 20 aa, Homo sapiens
VGWYRPPFSRVVHLYRNGKD

SEQ ID NO: 38 - protein, 26 aa, Homo sapiens
LKVEDPFYWVSPGVLVLLAVLPVLLL

SEQ ID NO: 39 - protein, 638 aa, Homo sapiens
MAGGSATTWGYPVALLLLVATLGLGRWLQPDPGLPGLRHSYDCGIKGMQLLVFPRPGQTLRFKVVDEFGNRFDVNNCSICYHWVTSRPQEPAVFSADYRGCHVLEKDGRFHLRVFMEAVLPNGRVDVAQDATLICPKPDPSRTLDSQLAPPAMFSVSTPQTLSFLPTSGHTSQGSGHAFPSPLDPGHSSVHPTPALPSPGPGPTLATLAQPHWGTLEHWDVNKRDYIGTHLSQEQCQVASGHLPCIVRRTSKEACQQAGCCYDNTREVPCYYGNTATVQCFRDGYFVLVVSQEMALTHRITLANIHLAYAPTSCSPTQHTEAFVVFYFPLTHCGTTMQVAGDQLIYENWLVSGIHIQKGPQGSITRDSTFQLHVRCVFNASDFLPIQASIFPPPSPAPMTQPGPLRLELRIAKDETFSSYYGEDDYPIVRLLREPVHVEVRLLQRTDPNLVLLLHQCWGAPSANPFQQPQWPILSDGCPFKGDSYRTQMVALDGATPFQSHYQRFTVATFALLDSGSQRALRGLVYLFCSTSACHTSGLETCSTACSTGTTRQRRSSGHRNDTARPQDIVSSPGPVGFEDSYGQEPTLGPTDSNGNSSLRPLLWAVLLLPAVALVLGFGVFVGLSQTWAQKLWESNRQ

SEQ ID NO: 40 - protein, 745 aa, Homo sapiens
MACRQRGGSWSPSGWFNAGWSTYRSISLFFALVTSGNSIDVSQLVNPAFPGTVTCDEREITVEFPSSPGTKKWHASVVDPLGLDMPNCTYILDPEKLTLRATYDNCTRRVHGGHQMTIRVMNNSAALRHGAVMYQFFCPAMQVEETQGLSASTICQKDFMSFSLPRVFSGLADDSKGTKVQMGWSIEVGDGARAKTLTLPEAMKEGFSLLIDNHRMTFHVPFNATGVTHYVQGNSHLYMVSLKLTFISPGQKVIFSSQAICAPDPVTCNATHMTLTIPEFPGKLKSVSFENQNIDVSQLHDNGIDLEATNGMKLHFSKTLLKTKLSEKCLLHQFYLASLKLTFLLRPETVSMVIYPECLCESPVSIVTGELCTQDGFMDVEVYSYQTQPALDLGTLRVGNSSCQPVFEAQSQGLVRFHIPLNGCGTRYKFEDDKVVYENEIHALWTDFPPSKISRDSEFRMTVKCSYSRNDMLLNINVESLTPPVASVKLGPFTLILQSYPDNSYQQPYGENEYPLVRFLRQPIYMEVRVLNRDDPNIKLVLDDCWATSTMDPDSFPQWNVVVDGCAYDLDNYQTTFHPVGSSVTHPDHYQRFDMKAFAFVSEAHVLSSLVYFHCSALICNRLSPDSPLCSVTCPVSSRHRRATGATEAEKMTVSLPGPILLLSDDSSFRGVGSSDLKASGSSGEKSRSETGEEVGSRGAMDTKGHKTAGDVGSKAVAAVAAFAGVVATLGFIYYLYEKRTVSNH

SEQ ID NO: 41 - protein, 373 aa, Homo sapiens
MVMVSKDLFGTGKLIRAADLTLGPEACEPLVSMDTEDVVRFEVGLHECGNSMQVTDDALVYSTFLLHDPRPVGNLSIVRTNRAEIPIECRYPRQGNVSSQAILPTWLPFRTTVFSEEKLTFSLRLMEENWNAEKRSPTFHLGDAAHLQAEIHTGSHVPLRLFVDHCVATPTPDQNASPYHTIVDFHGCLVDGLTDASSAFKVPRPGPDTLQFTVDVFHFANDSRNMIYITCHLKVTLAEQDPDELNKACSFSKPSNSWFPVEGSADICQCCNKGDCGTPSHSRRQPHVMSQWSRSASRNRRHVTEEADVTVGPLIFLDRRGDHEVEQWALPSDTSVVLLGVGLAVVVSLTLTAVILVLTRRCRTASHPVSASE

SEQ ID NO: 42 - protein, 13 aa, Homo sapiens
NSSSSQFQIHGPR

SEQ ID NO: 43 - protein, 8 aa, Homo sapiens
QFQIHGPR

SEQ ID NO: 44 - protein, 11 aa, Homo sapiens
NSSSSQFQIHG

SEQ ID NO: 45 - protein, 1937 aa, Homo sapiens [sequence omitted]

SEQ ID NO: 46 - protein, 30 aa, Homo sapiens
  N-term acetylated
SLKLMATLFSTYASADTGDSGKGKGGKKKG

SEQ ID NO: 47 - protein, 14 aa, Homo sapiens
GQFIDSGKAGAEKL

SEQ ID NO: 48 - protein, 14 aa, Homo sapiens
DECSELKKDIDDLE

SEQ ID NO: 49 - protein, 20 aa, Coxsackievirus
MGAQVSTQKTGAHETSLSAS

SEQ ID NO: 50 - protein, 20 aa, Coxsackievirus
GNSIIHYTNINYYKDAASNS

SEQ ID NO: 51 - protein, 20 aa, Coxsackievirus
NYYKDAASNSANRQDFTQDP

SEQ ID NO: 52 - protein, 20 aa, Coxsackievirus
ANRQDFTQDPSKFTEPVKDV

SEQ ID NO: 53 - protein, 20 aa, Coxsackievirus
SKFTEPVKDVMIKSLPALNS

SEQ ID NO: 54 - protein, 20 aa, Coxsackievirus
MIKSLPALNSPTVEECGYSD

SEQ ID NO: 55 - protein, 20 aa, Coxsackievirus
PTVEECGYSDRVRSITLGNS

SEQ ID NO: 56 - protein, 20 aa, Coxsackievirus
RVRSITLGNSTITTQECANV

SEQ ID NO: 57 - protein, 20 aa, Coxsackievirus
TITTQECANVVVGYGVWPDY

SEQ ID NO: 58 - protein, 20 aa, Coxsackievirus
LSDEEATAEDQPTQPDVATC

SEQ ID NO: 59 - protein, 20 aa, Coxsackievirus
QPTQPDVATCRFYTLNSVKW

SEQ ID NO: 60 - protein, 20 aa, Coxsackievirus
RFYTLNSVKWEMQSAGWWWK

SEQ ID NO: 61 - protein, 20 aa, Coxsackievirus
FPDALSEMGLFGQNMQYHYL

SEQ ID NO: 62 - protein, 20 aa, Coxsackievirus
FGQNMQYHYLGRSGYTIHVQ

SEQ ID NO: 63 - protein, 20 aa, Coxsackievirus
GRSGYTIHVQCNASKFHQGC

SEQ ID NO: 64 - protein, 20 aa, Coxsackievirus
CNASKFHQGCLLVVCVPEAE

SEQ ID NO: 65 - protein, 20 aa, Coxsackievirus
AYGDLCGGETAKSFEQNAAT

SEQ ID NO: 66 - protein, 20 aa, Coxsackievirus
AKSFEQNAATGKTAVQTAVC

SEQ ID NO: 67 - protein, 20 aa, Coxsackievirus
GKTAVQTAVCNAGMGVGVGN

SEQ ID NO: 68 - protein, 20 aa, Coxsackievirus
LTIYPHQWINLRTNNSATIV

SEQ ID NO: 69 - protein, 20 aa, Coxsackievirus
LRTNNSATIVMPYINSVPMD

SEQ ID NO: 70 - protein, 20 aa, Coxsackievirus
MPYINSVPMDNMFRHNNFTL

SEQ ID NO: 71 - protein, 20 aa, Coxsackievirus
NMFRHNNFTLMIIPFAPLDY

SEQ ID NO: 72 - protein, 20 aa, Coxsackievirus
YNGLRLAGHQGLPTMLTPGS

SEQ ID NO: 73 - protein, 20 aa, Coxsackievirus
SPSAMPQFDVTPEMNIPGQV

SEQ ID NO: 74 - protein, 20 aa, Coxsackievirus
TPEMNIPGQVRNLMEIAEVD

SEQ ID NO: 75 - protein, 20 aa, Coxsackievirus
RNLMEIAEVDSVVPINNLKA

SEQ ID NO: 76 - protein, 20 aa, Coxsackievirus
SVVPINNLKANLMTMEAYRV

SEQ ID NO: 77 - protein, 20 aa, Coxsackievirus
NLMTMEAYRVQVRSTDEMGG

SEQ ID NO: 78 - protein, 20 aa, Coxsackievirus
QVRSTDEMGGQIFGFPLQPG

SEQ ID NO: 79 - protein, 20 aa, Coxsackievirus
QIFGFPLQPGASSVLQRTLL

SEQ ID NO: 80 - protein, 20 aa, Coxsackievirus
ASSVLQRTLLGEILNYYTHW

SEQ ID NO: 81 - protein, 20 aa, Coxsackievirus
GEILNYYTHWSGSLKLTFVF

SEQ ID NO: 82 - protein, 20 aa, Coxsackievirus
SGSLKLTFVFCGSAMATGKF

SEQ ID NO: 83 - protein, 20 aa, Coxsackievirus
DDKYTASGFISCWYQTNVIV

SEQ ID NO: 84 - protein, 20 aa, Coxsackievirus
MCFVSACNDFSVRMLRDTQF

SEQ ID NO: 85 - protein, 20 aa, Coxsackievirus
LRRKMEMFTYIRCDMELTFV

SEQ ID NO: 86 - protein, 20 aa, Coxsackievirus
VPTSVNDYVWQTSTNPSIFW

SEQ ID NO: 87 - protein, 20 aa, Coxsackievirus
QTSTNPSIFWTEGNAPPRMS

SEQ ID NO: 88 - protein, 20 aa, Coxsackievirus
TEGNAPPRMSIPFMSIGNAY

SEQ ID NO: 89 - protein, 20 aa, Coxsackievirus
IPFMSIGNAYTMFYDGWSNF

SEQ ID NO: 90 - protein, 20 aa, Coxsackievirus
SRDGIYGYNSLNNMGTIYAR

SEQ ID NO: 91 - protein, 20 aa, Coxsackievirus
LNNMGTIYARHVNDSSPGGL

SEQ ID NO: 92 - protein, 20 aa, Coxsackievirus
HVNDSSPGGLTSTIRIYFKP

SEQ ID NO: 93 - protein, 20 aa, Coxsackievirus
SVNFDVEAVTAERASLITTG

SEQ ID NO: 94 - protein, 19 aa, Coxsackievirus
GLKTENEGLKTENEGLKTE

SEQ ID NO: 95 - protein, 18 aa, Coxsackievirus
KKEHEAENDKLKQQRDTL

SEQ ID NO: 96 - protein, 18 aa, Coxsackievirus
VKDKIAKEQENKETIGTL

SEQ ID NO: 97 - protein, 18 aa, Coxsackievirus
TIGTLKKILDETVKDKIA

SEQ ID NO: 98 - protein, 18 aa, Coxsackievirus
IGTLKKILDETVKDKLAK

SEQ ID NO: 99 - protein, 16 aa, Coxsackievirus
KGLRRDLDASREAKKQ

SEQ ID NO: 100 - protein, 18 aa, Coxsackievirus
LKTKNEGLKTENEGLKTE

SEQ ID NO: 101 - protein, 18 aa, Coxsackievirus
KKEHEAENDKLKQQRDTL

SEQ ID NO: 102 - protein, 18 aa, Coxsackievirus
QRDTLSTQKETLEREVQN

SEQ ID NO: 103 - protein, 18 aa, Coxsackievirus
REVQNTQYNNETLKIKNG

SEQ ID NO: 104 - protein, 18 aa, Coxsackievirus
KIKNGDLTKELNKTRQEL

SEQ ID NO: 105 - protein, 19 aa, Coxsackievirus
TRQELANKQQESKENEKAL

SEQ ID NO: 106 - protein, 18 aa, Coxsackievirus
TIGTLKKILDETVKDKIA

SEQ ID NO: 107 - protein, 18 aa, Coxsackievirus
IGTLKKILDETVKDKLAK

SEQ ID NO: 108 - protein, 35 aa, Coxsackievirus
AVTRGTINDPQRAKEALDKYELENHDLKTKNEGLK

SEQ ID NO: 109 - protein, 27 aa, Coxsackievirus
KTKNEGLKTENEGLKTENEGLKTENEG

SEQ ID NO: 110 - protein, 16 aa, Coxsackievirus
LKTEKKEHEAENDKLK

SEQ ID NO: 111 - protein, 30 aa, Coxsackievirus
DLTKELNKTRQELANKQQESKENEKAINEL

SEQ ID NO: 112 - protein, 30 aa, Coxsackievirus
LEKTVKDKIAKEQENKETIGTLKKILDETV

SEQ ID NO: 113 - protein, 16 aa, Coxsackievirus
TIGTLKKILDETVKDK

SEQ ID NO: 114 - protein, 21 aa, Streptococcus pyogenes
ISDASRKGLRRDLDASREAKK

SEQ ID NO: 115 - protein, 20 aa, Streptococcus pyogenes
DASREAKKQVEKAIEEANSK

SEQ ID NO: 116 - protein, 20 aa, Streptococcus pyogenes
ALEEANSKLAALEKLNKELE

SEQ ID NO: 117 - protein, 31 aa, Streptococcus pyogenes
ELEESKKLTEKEKAELQAKLEAEAKQLKEQL

SEQ ID NO: 118 - protein, 30 aa, Streptococcus pyogenes
AKQAEELAKLRAGKASDSQTPDTKPGNKAV

SEQ ID NO: 119 - protein, 37 aa, Streptococcus pyogenes
VPGKGQAPQAGTKPNQNKAPMKETKRQLPSTGETANP

SEQ ID NO: 120 - protein, 19 aa, Streptococcus pyogenes
LRRDLDASREAKKQVEKAI

SEQ ID NO: 121 - protein, 20 aa, Streptococcus pyogenes
AKKQVEKALEEANSKLAALE

SEQ ID NO: 122 - protein, 20 aa, Streptococcus pyogenes
KLTEKEKAELQAKLEAEAKA

SEQ ID NO: 123 - protein, 20 aa, Streptococcus pyogenes
QAKLEAEAKALKEQLAKQAE

SEQ ID NO: 124 - protein, 20 aa, Streptococcus pyogenes
LKEQLAKQAEELAKLRAGKA

SEQ ID NO: 125 - protein, 25 aa, Streptococcus pyogenes
TVTRGTISDPQRAKEALDKYELENH

SEQ ID NO: 126 - protein, 24 aa, Streptococcus pyogenes
DKLKQQRDTLSTQKETLEREVQNI

SEQ ID NO: 127 - protein, 15 aa, Streptococcus pyogenes
ETIGTLKKILDETVK

SEQ ID NO: 128 - protein, 18 aa, Streptococcus pyogenes
AVTRGTINDPQRAKEALD

SEQ ID NO: 129 - protein, 18 aa, Streptococcus pyogenes
KEALDKYELENHDLKTKN

SEQ ID NO: 130 - protein, 18 aa, Streptococcus pyogenes
LKTKNEGLKTENEGLKTE

SEQ ID NO: 131 - protein, 19 aa, Streptococcus pyogenes
GLKTENEGLKTENEGLKTE

SEQ ID NO: 132 - protein, 18 aa, Streptococcus pyogenes
KKEHEAENDKLKQQRDTL

SEQ ID NO: 133 - protein, 18 aa, Streptococcus pyogenes
QRDTLSTQKETLEREVQN

SEQ ID NO: 134 - protein, 18 aa, Streptococcus pyogenes
REVQNTQYNNETLKIKNG

SEQ ID NO: 135 - protein, 18 aa, Streptococcus pyogenes
KIKNGDLTKELNKTRQEL

SEQ ID NO: 136 - protein, 19 aa, Streptococcus pyogenes
TRQELANKQQESKENEKAL

SEQ ID NO: 137 - protein, 18 aa, Streptococcus pyogenes
ENEKALNELLEKTVKDKI

SEQ ID NO: 138 - protein, 18 aa, Streptococcus pyogenes
VKDKIAKEQENKETIGTL

SEQ ID NO: 139 - protein, 18 aa, Streptococcus pyogenes
TIGTLKKILDETVKDKIA

SEQ ID NO: 140 - protein, 18 aa, Streptococcus pyogenes
KDKIAKEQENKETIGTLK

SEQ ID NO: 141 - protein, 18 aa, Streptococcus pyogenes
IGTLKKILDETVKDKLAK

SEQ ID NO: 142 - protein, 18 aa, Streptococcus pyogenes
DKLAKEQKSKQNIGALKQ

SEQ ID NO: 143 - protein, 18 aa, Streptococcus pyogenes
GALKQELAKKDEANKISD

SEQ ID NO: 144 - protein, 18 aa, Streptococcus pyogenes
NKISDASRKGLRRDLDAS

SEQ ID NO: 145 - protein, 18 aa, Streptococcus pyogenes
DLDASREAKKQLEAEHQK

SEQ ID NO: 146 - protein, 18 aa, Streptococcus pyogenes
AEHQKLEEQNKISEASRK

SEQ ID NO: 147 - protein, 18 aa, Streptococcus pyogenes
EASRKGLRRDLDASREAK

SEQ ID NO: 148 - protein, 18 aa, Streptococcus pyogenes
SREAKKQLEAEQQKLEEQ

SEQ ID NO: 149 - protein, 18 aa, Streptococcus pyogenes
KLEEQNKISEASRKGLRR

SEQ ID NO: 150 - protein, 16 aa, Streptococcus pyogenes
KGLRRDLDASREAKKQ

SEQ ID NO: 151 - protein, 1487 aa, Homo sapiens
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 152 - protein, 8 aa, Homo sapiens
AGERGPPG

SEQ ID NO: 153 - protein, 15 aa, Homo sapiens
AGGFDEKAGGAQLGV

SEQ ID NO: 154 - protein, 11 aa, Homo sapiens
VGPAGGPGFPG

SEQ ID NO: 155 - protein, 12 aa, Homo sapiens
  MOD_RES (5)..(5): 3, 5, 3′, 5′-Tetraiodothyronine (thyroxine)
NIFEXQVDAQPL

SEQ ID NO: 156 - protein, 22 aa, Homo sapiens
  MOD_RES (10)..(10): 3, 5, 3′, 5′-Tetraiodothyronine (thyroxine)
YSLEHSTDDXASFSRALENATR

SEQ ID NO: 157 - protein, 20 aa, Homo sapiens
  MOD_RES (10)..(10): 3, 5, 3′, 5′-Tetraiodothyronine (thyroxine)
RALENATRDXFIICPIIDMA

SEQ ID NO: 158 - protein, 14 aa, Homo sapiens
  MOD_RES (12)..(12): 3, 5, 3′, 5′-Tetraiodothyronine (thyroxine)
LLSLQEPGSKTXSK

SEQ ID NO: 159 - protein, 16 aa, Homo sapiens
  MOD_RES (7)..(7): 3, 5, 3′, 5′-Tetraiodothyronine (thyroxine)
EHSTDDXASFSRALEN

SEQ ID NO: 160 - protein, 14 aa, Homo sapiens
LKKRGILSPAQLLS

SEQ ID NO: 161 - protein, 16 aa, Homo sapiens
SGVIARAAEIMETSIQ

SEQ ID NO: 162 - protein, 14 aa, Homo sapiens
PPVREVTRHVIQVS

SEQ ID NO: 163 - protein, 15 aa, Homo sapiens
PRQQMNGLTSFLDAS

SEQ ID NO: 164 - protein, 15 aa, Homo sapiens
LTALHTLWLREHNRL

SEQ ID NO: 165 - protein, 15 aa, Homo sapiens
HNRLAAALKALNAHW

SEQ ID NO: 166 - protein, 14 aa, Homo sapiens
ARKVVGALHQIITL

SEQ ID NO: 167 - protein, 16 aa, Homo sapiens
LPGLWLHQAFFSPWTL

SEQ ID NO: 168 - protein, 17 aa, Homo sapiens
MNEELTERLFVLSNSST

SEQ ID NO: 169 - protein, 11 aa, Homo sapiens
LDLASINLQRG

SEQ ID NO: 170 - protein, 16 aa, Homo sapiens
RSVADKILDLYKHPDN

SEQ ID NO: 171 - protein, 14 aa, Homo sapiens
IDVWLGGLAENFLP

SEQ ID NO: 172 - protein, 12 aa, Homo sapiens
QQQHERRKQERK

SEQ ID NO: 173 - protein, 14 aa, Homo sapiens
PTKEIEIQVDWNSE

SEQ ID NO: 174 - protein, 780 aa, Homo sapiens
MAAPGGRSEPPQLPEYSCSYMVSRPVYSELAFQQQHERRLQERKTLRESLAKCCSCSRKRAFGVLKTLVPILEWLPKYRVKEWLLSDVISGVSTGLVATLQGMAYALLAAVPVGYGLYSAFFPILTYFIFGTSRHISVGPFPVVSLMVGSVVLSMAPDEHFLVSSSNGTVLNTTMIDTAARDTARVLIASALTLLVGIIQLIFGGLQIGFIVRYLADPLVGGFTTAAAFQVLVSQLKIVLNVSTKNYNGVLSIIYTLVEIFQNIGDTNLADFTAGLLTIVVCMAVKELNDRFRHKIPVPIPIEVIVTIIATAISYGANLEKNYNAGIVKSIPRGFLPPELPPVSLFSEMLAASFSIAVVAYAIAVSVGKVYATKYDYTIDGNQEFIAFGISNIFSGFFSCFVATTALSRTAVQESTGGKTQVAGIISAAIVMIAILALGKLLEPLQKSVLAAVVIANLKGMFMQLCDIPRLWRQNKIDAVIWVFTCIVSIILGLDLGLLAGLIFGLLTVVLRVQFPSWNGLGSIPSTDIYKSTKNYKNIEEPQGVKILRFSSPIFYGNVDGFKKCIKSTVGFDAIRVYNKRLKALRKIQKLIKSGQLRATKNGIISDAVSTNNAFEPDEDIEDLEELDIPTKEIEIQVDWNSELPVKVNVPKVPIHSLVLDCGAISFLDVVGVRSLRVIVKEFQRIDVNVYFASLQDYVIEKLEQCGFFDDNIRKDTFFLTVHDAILYLQNQVKSQEGQGSILETITLIQDCKDTLELIETELTEEELDVQDEAMRTLAS

SEQ ID NO: 175 - protein, 457 aa, Homo sapiens
MEPWPLLLLFSLCSAGLVLGSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPLYFIVNVIIPCLLFSFLTGLVFYLPTDSGEKMTLSISVLLSLTVFLLVIVELIPSTSSAVPLIGKYMLFTMVFVIASIIITVIVINTHHRSPSTHVMPNWVRKVFIDTIPNIMFFSTMKRPSREKQDKKIFTEDIDISDISGKPGPPPMGFHSPLIKHPEVKSAIEGIKYIAETMKSDQESNNAAAEWKYVAMVMDHILLGVFMLVCIIGTLAVFAGRLIELNQQG

SEQ ID NO: 176 - protein, 403 aa, Homo sapiens
LVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPLYFIVNVIIPCLLFSFLTGLVFYLPTDSGEKMTLSISVLLSLTVFLLVIVELIPSTSSAVPLIGKYMLFTMVFVIASIIITVIVINTHHRSPSTHVMPNWVRKVFIDTIPNIMFFSTMKRPSREKQDKKIFTEDIDISDISGKPGPPPMGFHSPLIKHPEVKSAIEGIKYIAETMKSDQESNNAAAEWKYVAMVMDHI

SEQ ID NO: 177 - protein, 8 aa, Homo sapiens
EIIVTHFP

SEQ ID NO: 178 - protein, 30 aa, Homo sapiens
WTPPAIFKSYCEIIVTHFPFDEQNCSMKLG

SEQ ID NO: 179 - protein, 15 aa, Homo sapiens
EIIVTHFPFDEQNCS

SEQ ID NO: 180 - protein, 14 aa, Homo sapiens
IFKSYCEIIVTHFP

SEQ ID NO: 181 - protein, 181 aa, Homo sapiens
MEPWPLLLLFSLCSAGLVLGSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPE

SEQ ID NO: 182 - protein, 437 aa, Homo sapiens
MEPWPLLLLFSLCSAGLVLGSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPLYFIVNVIIPCLLFSFLTGLVFYLPTDSGEKMTLSISVLLSLTVFLLVIVELIPSTSSAVPLIGKYMLFTMVFVIASIIITVIVINTHHRSPSTHVMPNWVRKVFIDTIPNIMFFSTMKRPSREKQDKKIFTEDIDISDISGKPGPPPMGFHSPLIKHPEVKSAIEGIKYIAETMKSDQESNNAAAEWKYVAMVMDHILLGVFMLV

SEQ ID NO: 183 - protein, 20 aa, Homo sapiens
VQHAPLEMGPQPRAEATWQF

SEQ ID NO: 184 - protein, 20 aa, Homo sapiens
GFLGELTSSEVATEVPFRLM

SEQ ID NO: 185 - protein, 20 aa, Homo sapiens
VATEVPFRLMHPQPEDPAKE

SEQ ID NO: 186 - protein, 20 aa, Homo sapiens
YLLTSHRTATAAEEFAFLMQ

SEQ ID NO: 187 - protein, 17 aa, Gallus gallus
ISQAVHAAHAEINEAGR

SEQ ID NO: 188 - protein, 15 aa, Pulex irritans
GPDWKVSKECKDPNN

SEQ ID NO: 189 - protein, 15 aa, Pulex irritans
QEKEKCMKFCKKVCK

SEQ ID NO: 190 - protein, 5 aa, Homo sapiens
  MOD_RES (1)..(1): Gln or Arg
XXRAA

SEQ ID NO: 191 - protein, 15 aa, Homo sapiens
SHLVEALYLVCGERG

SEQ ID NO: 192 - protein, 110 aa, Homo sapiens; identical to SEQ ID NO: 4